(19) United States
(12) Patent Application Publication
(10) Pub. No.: US 2020/0031825 A1
(43) Pub. Date: Jan. 30, 2020

(54) HETEROAROMATIC-FUSED IMIDAZOLYL AMIDES, COMPOSITIONS AND USES THEREOF AS STING AGONISTS

(71) Applicant: Trillium Therapeutics Inc., Mississauga (CA)

(72) Inventors: Abdelmalik Slassi, Mississauga (CA); Peter DOVE, Burlington (CA); Zezhou WANG, Oakville (CA); Hoi Ying LIN, Oakville (CA); David Alexander ROSA, Toronto (CA); Jeffrey Todd WINSTON, Richfield, MN (US)

(73) Assignee: Trillium Therapeutics Inc., Mississauga (CA)

(21) Appl. No.: 16/593,744

(22) Filed: Oct. 4, 2019

Related U.S. Application Data
(63) Continuation-in-part of application No. PCT/CA2019/050944, filed on Jul. 9, 2019.
(60) Provisional application No. 62/696,171, filed on Jul. 10, 2018.

(51) Int. Cl. C07D 471/04; C07D 495/04; C07D 519/00; A61P 35/00

(52) U.S. Cl. CPC C07D 471/04; C07D 495/04; A61K 47/6803; A61P 35/00; C07D 519/00

(57) ABSTRACT

Compounds of Formula I and II have been prepared for use in the treatment of diseases, disorders or conditions treatable by activation of the stimulator of interferon genes (STING) adaptor protein, such as in the treatment or prevention of cancers.

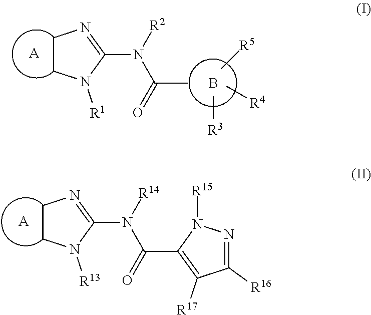

### RELATED APPLICATIONS

The present application is a continuation-in-part of co-pending International patent application no. PCT/CA2019/050944 filed Jul. 9, 2019 which claims the benefit of priority of U.S. provisional patent application No. 62/696,171 filed on Jul. 10, 2018, the contents of both of which are incorporated herein by reference in their entirety.

### FIELD

The present application relates to bicyclic amide compounds comprising at least one heteroaromatic ring fused to an imidazolyl ring, to processes for their preparation, to compositions comprising them, and to their use in therapy. More particularly, it relates to compounds useful in the treatment of diseases, disorders or conditions treatable by activation of the stimulator of interferon genes (STING) adaptor protein, such as in the treatment or prevention of a number of different cancers.

### BACKGROUND

Mammals, as well as other eukaryotic organisms, have developed specific defense mechanisms to deal with the constant assault of invading microorganisms. In the case of vertebrates, the immune system, comprised of two arms dealing with innate and adaptive immunity, has evolved to eliminate noxious pathogens. As a first line of defense, the innate immune system deploys specific recognition receptors to detect molecular patterns belonging to extracellular or intercellular pathogens [Cell, 2010, 349:1232-1236]. Recognition of extracellular pathogens is accomplished primarily through Toll-like receptors (TLRs) and C-type lectin receptors (CLRs) [Biochem Biophys Res Commun, 2009, 388:621-625]. Various recognition proteins are used to sense intracellular pathogens. For example, NOD-like receptors respond to a variety of bacterial peptidoglycans including muramyl dipeptide, a motif commonly associated with Streptococcus pneumoniae and Mycobacterium tuberculosis [Annu Rev Pathol. 4: 365-398]. Receptors of the Rig-1-like (RLR) family survey the cytosol for viral RNA and induce the production of type I interferons and cytokines upon activation [Curr Opin Immunol. 2015, 32: 48-53]. Finally, cyclic GMP-AMP (cGAMP) synthase acts as a sensor of cytosolic DNA, capable of initiating the production of interferons through the activation of the STING (stimulator of interferon genes) adaptor protein [Mol. Cell, 2015, 54: 289-296].

STING, is a pattern recognition receptor and adaptor protein that mediates an innate immune response following detection of cytoplasmic DNA. STING-dependent signaling is involved in certain autoimmune diseases and cancer immunology. STING, otherwise known as TMEM173, MPYS, MITA and ERIS, is involved in mobilizing the host innate immune system in response to aberrant DNA, including cyclic dinucleotides (CDNs), located in the cytosol [Nature, 2008, 455: 674-678]. As the occurrence of cytosolic DNA is tightly regulated during the cell cycle, its presence often indicates severe cell damage and dysregulation, as well as infection by invading pathogens, such as DNA viruses or bacteria. Certain pathological conditions have been shown to be caused by the “leakage” of self-DNA from the nucleus of the host cells. Indeed, autoinflammatory diseases such as systemic lupus erythematosus (SLE) [PNAS, 2015, 112(7): E710-E717] and Aicardi-Goutieres syndrome (AGS) [J Exp Med, 2016, 213(3): 329-336] serve as poignant examples of the host immune system reacting to self-DNA. While immune system recognition of self-DNA is undesirable in a general context, the detection of tumor-derived DNA can provide a vehicle for the innate sensing of tumors and activation of anti-tumor immunity.

STING is comprised of four transmembrane (TM) domains which function to anchor the protein to the ER membrane, a carboxyterminal (CT) domain which projects into the cytoplasm and a dimerization domain (DD) [Nat. Struct. Mol. Biol. 2012, 19, 722-724]. CDNs bind to a central U-shaped groove formed upon STING homodimerization [Immunity 2012, 36, 1073-1086]. Binding of its cognate ligand induces a conformational change in STING that is postulated to form a highly structured four-stranded antiparallel β sheet which restricts access to the binding groove of the dimer and forms a scaffold upon which IRF3 can be phosphorylated by TBK1. The phosphorylation of STING by TBK1 which in-turn recruits IRF3 to be phosphorylated by the aforementioned kinase is an important regulatory mechanism, ensuring the controlled activation of IRF3. Phosphorylated IRF3 forms a homodimer capable of entering the nucleus, where it functions in conjunction with NF-kB to upregulate the expression of type I interferons and other immunomodulatory molecules [Science, 2015, 347, aaa2630].

Mobilization of the adaptive immune system is beneficial for the control and eradication of cancer cells [EMBO J. 2016, 35, 1385-1399]. A vast array of immunotherapeutic strategies geared towards stimulating the adaptive immune response against cancers, such as checkpoint blockade via PD-1/PD-L1 or CTLA4, are currently being evaluated in a clinical setting. However, the exact role dendritic cells (DCs) play in fostering an adaptive immune response against tumour cells remains unclear. It appears that type I IFN production by CD8α+ DCs is required for the cross-presentation of tumor antigen and priming of tumour-specific T cells [Science, 2015, 350, 568-571]. Furthermore, the induction of type I IFN is thought to be independent of TLR or RLR-mitochondrial antiviral signalling (MAVS) protein pathways. It has been shown that the STING pathway is responsible for spontaneous antitumour T cell responses following radiotherapy [Curr. Opin. Immunol. 2015, 32, 7-12]. As a corollary, STING-deficient mice are unable to generate efficient antitumour T cell responses in the context of melanoma. Furthermore, checkpoint inhibitor mediated T cell responses are abolished in STING-deficient mice, suggesting a role for STING in therapies involving checkpoint blockade [J. Immunol. 2014, 193, 2394-2404]. One well accepted hypothesis for the role of STING in tumour immunity involves activation of the protein through the efferocytosis of tumour cells by CD8α+ DCs. Once engulfed, tumour derived DNA triggers the STING pathway resulting in the upregulation of type I IFNs. The IFNs produced stimulate the production of additional factors required for DC cross-presentation and ultimately T cell sensitization to tumour antigens.

The role of STING in mediating tumour specific T cell responses has garnered a significant amount of interest as a potential immuno-oncology target for the treatment of cancer. The first clinical candidate to be described was the small molecule, non-dinucleotide, 5,6-dimethyl-xanthenone-4-acetic acid (DMXAA or Vadimezan, see FIG. 1). DMXAA was previously characterized as a vascular-disrupting agent exhibiting anti-tumour activity and was later found to be a potent agonist of mouse STING (Clin. Science, 2012, 122(10), 449-465). Unfortunately, the observed agonist activity did not translate to human STING and DMXAA was removed from a Phase III clinical trial due to lack of efficacy. Despite this initial failure, GSK and Merck independently disclosed novel small molecule STING agonists that are more efficacious than cGAMP. A number of CDN based STING agonists are being pursued as potential therapeutics. Aduro Biotech was the first to progress into clinical trials with ADU-S100 (see FIG. 1). Aduro's clinical candidate is a modified cGMP incorporating a phosphorothioate linkage between the purine bases of the CDN that is resistant to phosphodiesterase mediated cleavage [Sci. Trans. Med. 2015, 7, 283ra52]. Currently, ADU-S100 is being evaluated in Phase I clinical trials for cutaneously-accessible tumors, including breast, head-and-neck, and renal cell cancers as well as lymphoma and melanoma [US Clinical Trial Identifier: NCT02675439]. Furthermore, the combinations of ADU-S100 and programmed cell death protein 1 (PD1) biologic PDR001 is being assessed in patients with metastatic solid tumors [US Clinical Trial Identifier NCT03172936]. By extension, similar STING agonists have been shown to be efficacious in tumours resistant to anti-PD1 blockade therapies [J. Exp. Med. 2016, 213, 329-336]. Both Merck and GSK have also adopted the use of phosphorothioate linkages for their CDN platforms and have explored the fluorination of the adjoining ribose sugar residues [PCT Patent Application Publication No.: WO 2017/093933] as well as substitution to the bridging carbinol group [PCT Patent Application Publication No.: WO 2017/027645]. A substantial amount of research has gone into developing CDN STING agonists, however, further development is still hampered by limited chemical stability and cell permeability. The limitations of CDNs being as such, novel ways of introducing CDN based STING agonists into the site of action, such as by nanoparticle [Nanomed: Nanotech, Bio and Med. 2018, 14(2):237-246] or potentially via viral vectors [Science 2015, 349:1232-1236], are being actively explored. Various benzimidazolyl amide compounds have been described as modulators of STING (PCT Patent Application Publication Nos. WO 2017/175147 and WO 2017/175156). Anticancer drugs, such as cisplatin and etoposide, are known to exert their cytotoxic effects through the formation of DNA adducts resulting in cell death [Cell, 2007, 131, 873-886]. Furthermore, these drugs were found to increase cytosolic levels of nuclear DNA, resulting in activation of the STING pathway and induction of cytokine genes [J. Virol. 2016, 90, 7789-7797]. Therefore the anticancer properties of similar DNA modifying chemotherapeutics may be traced back, at least in part, to the STING pathway. Taken together, it is becoming more apparent that STING agonism may constitute a new immunotherapeutic strategy for the treatment of malignant disease.

### SUMMARY

A class of bicylic amide compounds comprising at least one heteroaromatic ring fused to an imidazolyl ring have been shown to have activity as activators STING. Therefore, compounds of Formula I have been prepared and found to be useful as STING agonists.

Accordingly, the present application includes a compound of Formula I or a pharmaceutically acceptable salt, solvate and/or prodrug thereof:

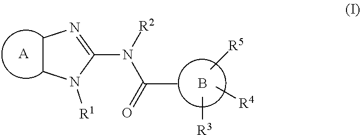

wherein:
Ring A is a 5 or 6-membered heteroaromatic ring comprising 1 to 3 heteroatoms selected from O, N and S that is substituted with at least one R⁶ and optionally substituted with one or two additional substituents independently selected from halo, C₁₋₆alkyl, C₂₋₆alkenyl, C₂₋₆alkynyl, OR^{a}, NR^{a}R^{b}, C₁₋₆alkyleneOR^{a}, C₁₋₆alkyleneNR^{a}R^{b}, OC₁₋₆alkyleneOR^{a}, OC₁₋₆alkyleneNR^{a}R^{b}, C₂₋₆alkenyleneOR^{a}, C₂₋₆alkenyleneNR^{a}R^{b}, OC₂₋₆alkenyleneOR^{a}, OC₂₋₆alkenyleneNR^{a}R^{b}, C₂₋₆alkynyleneOR^{a}, C₂₋₆alkynyleneNR^{a}R^{b}, OC₂₋₆alkynyleneOR^{a} and OC₂₋₆alkynyleneNR^{a}R^{b};
R^{a} and R^{b} are independently selected from H, C₁₋₆alkyl, C₁₋₆alkyleneOC₁₋₆alkyl, C₂₋₆alkenyleneOC₁₋₆alkyl, and C₂₋₆alkynyleneOC₁₋₆alkyl, or
R^{a} and R^{b} are joined to form, together with the atoms therebetween, a 3-10 membered saturated or unsaturated ring, optionally containing one additional heteroatom selected from NR^{s}, O, S, SO and SO₂, and optionally substituted with one or more of D, OH, NH₂, CHO, CO₂H, CONH₂, C₁₋₆alkyl, C₁₋₆alkenyl, C₁₋₆alkynyl C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl; R^{s} is selected from H, D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R¹ is selected from C₁₋₁₀alkyleneR⁷, C₂₋₁₀alkenyleneR⁷, C₂₋₁₀alkynyleneR⁷, C₃₋₁₀cycloalkyl, 4-10-membered heterocycloalkyl, 5-10-membered heteroaryl and C₆₋₁₀aryl, the latter 4 groups and each C₁₋₁₀alkylene, C₂₋₁₀alkenylene and C₂₋₁₀alkynylene being optionally substituted with 1-4 substituents independently selected from halo, nitro, C₁₋₆alkyleneR^{c}, C₁₋₆alkyleneOR^{c}, OC₁₋₆alkyleneOR^{c}, C₁₋₆alkyleneNR^{c}R^{d}, OC₁₋₆alkyleneNR^{c}R^{d}, OR^{c}, NR^{c}R^{d}, CO₂R^{c}, CONR^{c}R^{d}, SO₂NR^{c}R^{d}, OCONR^{c}R^{d}, COR^{e}, C₃₋₁₀cycloalkyl, 3-10-membered heterocycloalkyl, 5-10-membered heteroaryl and C₆₋₁₀aryl;
R^{c} is selected from H, C₁₋₆alkyl, COC₁₋₆alkyl, C₃₋₁₀cycloalkyl, 3-10-membered heterocycloalkyl, 5-10-membered heteroaryl and C₆₋₁₀aryl;
R^{d} is selected from H and C₁₋₆alkyl;
R^{e} is selected from H, C₁₋₆alkyl, C₃₋₁₀cycloalkyl, 3-10-membered heterocycloalkyl, 5-10-membered heteroaryl and C₆₋₁₀aryl;
R² is selected from H, C₁₋₆alkyl, C₂₋₆alkenyl and C₂₋₆alkynyl;
Ring B is a 5 or 6-membered heteroaromatic ring comprising 1 to 3 heteroatoms selected from O, N and S;
R³ is selected from H, C₁₋₆alkyl, C₃₋₁₀cycloalkyl and 3-10-membered heterocycloalkyl, the latter three groups being optionally substituted with halo, OR^{f}, NR^{f}R^{g}, COR^{f}, CO₂R^{f}, CONR^{f}R^{g}, SO₂NR^{f}R^{g}, NHSO₂R^{f} and OCONR^{f}R^{g}; R^{f} and R^{g} are independently selected from H and C₁₋₆alkyl;
R⁴ is selected from H, C₁₋₆alkyl, C₂₋₆alkenyl, C₂₋₆alkynyl and C₃₋₇cycloalkyl;
R⁵ is selected from H, halo and C₁₋₆alkyl;
R⁶ is selected from COR^{h}, CO₂R^{h}, CONR^{h}Rⁱ, OCONR^{h}Rⁱ, NHSO₂R^{h}, SO₂NR^{h}Rⁱ, NHCOR^{h} and NHCO₂R^{h},
R^{h} and Rⁱ are independently selected from H and C₁₋₆alkyl;
R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₁₀cycloalkyl, 4-10-membered heterocycloalkyl, 5-10-membered heteroaryl and C₆₋₁₀aryl, the latter 4 groups being optionally substituted with 1-4 substituents independently selected from halo, nitro, C₁₋₆alkyleneR^{m}, C₁₋₆alkyleneOR^{m}, OC₁₋₆alkyleneOR^{m}, C₁₋₆alkyleneNR^{m}Rⁿ, OC₁₋₆alkyleneNR^{m}Rⁿ, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl, or R⁷ is

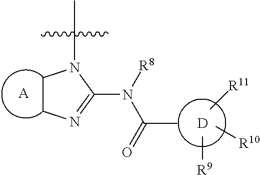

R^{j} is selected from H, C₁₋₆alkyl, COC₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, C₁₋₆alkyleneNR^{m}Rⁿ, OC₁₋₆alkyleneNR^{m}Rⁿ, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{k} is selected from H and C₁₋₆alkyl;
R^{l} is selected from H, C₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, C₁₋₆alkyleneNR^{m}Rⁿ, OC₁₋₆alkyleneNR^{m}Rⁿ, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{m} is selected from H and C₁₋₆alkyl;
Rⁿ is selected from H, C₁₋₆alkyl, C₃₋₁₀cycloalkyl, 4-10-membered heterocycloalkyl, 5-10-membered heteroaryl and C₆₋₁₀aryl;
R⁸, R⁹, R¹⁰ and R¹¹ are, independently, as defined for R², R³, R⁴ and R⁵, respectively;
Ring C is selected from phenyl and a 5 or 6-membered heteroaromatic ring comprising 1 to 3 heteroatoms selected from O, N and S, and Ring C is substituted with at least one R¹² and optionally substituted with one or two additional substituents independently selected from halo, C₁₋₆alkyl, C₂₋₆alkenyl, C₂₋₆alkynyl, OR^{o}, NR^{o}R^{p}, C₁₋₆alkyleneOR^{o}, C₁₋₆alkyleneNR^{o}R^{p}, OC₁₋₆alkyleneOR^{o}, OC₁₋₆alkyleneNR^{o}R^{p}, C₂₋₆alkenyleneOR^{o}, C₂₋₆alkenyleneNR^{o}R^{p}, OC₂₋₆alkenyleneOR^{o}, OC₂₋₆alkenyleneNR^{o}R^{p}, C₂₋₆alkynyleneOR^{o}, C₂₋₆alkynyleneNR^{o}R^{p}, OC₂₋₆alkynyleneOR^{o} and OC₂₋₆alkynyleneNR^{o}R^{p};
R^{o} and R^{p} are independently selected from H, C₁₋₆alkyl, C₁₋₆alkyleneOC₁₋₆alkyl, C₂₋₆alkenyleneOC₁₋₆alkyl, and C₂₋₆alkynyleneOC₁₋₆alkyl, or
R^{o} and R^{p} are joined to form, together with the atoms therebetween, a 3-10 membered saturated or unsaturated ring, optionally containing one additional heteroatom selected from NR^{t}, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R^{t} is selected from H, D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl; Ring D is a 5 or 6-membered heteroaromatic ring comprising 1 to 3 heteroatoms selected from O, N and S;
R¹² is selected from COR^{q}, CO₂R^{q}, CONR^{q}R^{r}, OCONR^{q}R^{r}, NHSO₂R^{q}, SO₂NR^{q}R^{r}, NHCOR^{q} and NHCO₂R^{q};
R^{q} and R^{r} are independently selected from H and C₁₋₆alkyl;
and
each alkyl, alkenyl, alkynyl, alkylene, alkenylene and alkynylene group is optionally fluoro-substituted

The present application also includes compounds that are a specific subset of compounds of Formula I, that is compounds of Formula II or a pharmaceutically acceptable salt, solvate and/or prodrug thereof:

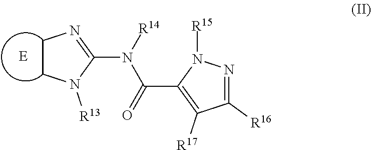

wherein:
Ring E is a 5 or 6-membered heteroaromatic ring comprising 1 to 3 heteroatoms selected from O, N and S that is substituted with at least one R¹⁸ and optionally substituted with one or two additional substituents independently selected from OC₁₋₆alkyleneNR¹⁹R²⁰, OC₂₋₆alkenyleneNR¹⁹R²⁰, OC₂₋₆alkynyleneNR¹⁹R²⁰, OC₁₋₆alkyleneNR²¹C₁₋₆alkyleneOC₁₋₆alkyl, OC₂₋₆alkenyleneNR²¹C₁₋₆alkyleneOC₁₋₆alkyl, OC₂₋₆alkynyleneNR²¹C₁₋₆alkyleneOC₁₋₆alkyl, OC₁₋₆alkyleneNR²¹C₂₋₆alkenyleneOC₁₋₆alkyl, OC₂₋₆alkenyleneNR²¹C₂₋₆alkenyleneOC₁₋₆alkyl, OC₂₋₆alkynyleneNR²¹C₂₋₆alkenyleneOC₁₋₆alkyl, OC₁₋₆alkyleneNR²¹C₂₋₆alkynyleneOC₁₋₆alkyl, OC₂₋₆alkenyleneNR²¹C₂₋₆alkynyleneOC₁₋₆alkyl, and OC₂₋₆alkynyleneNR²¹C₂₋₆alkynyleneOC₁₋₆alkyl;
R¹³ is selected from C₁₋₁₀alkyleneR²², C₂₋₁₀alkenyleneR²² and C₂₋₁₀alkynyleneR²²;
R¹⁴ is selected from H, C₁₋₆alkyl, C₂₋₆alkenyl and C₂₋₆alkynyl;
R¹⁵ is selected from H, C₁₋₆alkyl, C₃₋₁₀cycloalkyl and 3-10-membered heterocycloalkyl, the latter three groups being optionally substituted with halo, OR²³, NR²³R²⁴, COR²³, CO₂R²³, CONR²³R²⁴, SO₂NR²³R²⁴, NHSO₂R²³ and OCONR²³R²⁴;
R¹⁶ is selected from H, C₁₋₆alkyl, C₂₋₆alkenyl, C₂₋₆alkynyl and C₃₋₇cycloalkyl;
R¹⁷ is selected from H, halo and C₁₋₆alkyl;
R¹⁸ is selected from COR²⁵, CO₂R²⁵, CONR²⁵R²⁶, OCONR²⁵R²⁶, NHSO₂R²⁵, SO₂NR²⁵R²⁶, NHCOR²⁵ and NHCO₂R²⁵,
R¹⁹ and R²⁰ are joined to form, together with the nitrogen therebetween, a 3-6 membered saturated or unsaturated ring, optionally containing one additional heteroatom selected from NR²⁷, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl) or C₁₋₆alkyleneOC₁₋₆alkyl; or
R¹⁹ and R²⁰ are joined to form, together with the nitrogen therebetween, a 7-10 membered saturated or unsaturated ring, optionally containing one additional heteroatom selected from NR²⁷, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R²³, R²⁴, R²⁵ and R²⁶ are independently selected from H and C₁₋₆alkyl;
R²⁷ is selected from D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;

### R²² is

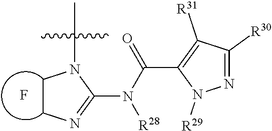

R²⁸, R²⁹, R³⁰ and R³¹ are, independently, as defined for R¹⁴, R¹⁵, R¹⁶ and R¹⁷, respectively;
Ring F is selected from phenyl and a 5 or 6-membered heteroaromatic ring comprising 1 to 3 heteroatoms selected from O, N and S, and Ring F is substituted with at least one R³² and substituted with one or two additional substituents independently selected from OC₁₋₆alkyleneNR³³R³⁴, OC₂₋₆alkenyleneNR³³R³⁴, OC₂₋₆alkynyleneNR³³R³⁴, OC₁₋₆alkyleneNR³⁵C₂₋₆alkenyleneOC₁₋₆alkyl, OC₁₋₆alkyleneNR³⁵C₁₋₆alkyleneOC₁₋₆alkyl, OC₂₋₆alkenyleneNR³⁵CG-6alkyleneOC₁₋₆alkyl, OC₂₋₆alkynyleneNR³⁵C₁₋₆alkyleneOC₁₋₆alkyl, OC₁₋₆alkyleneNR³⁵C₂₋₆alkenyleneOC₁₋₆alkyl, OC₂₋₆alkenyleneNR³⁵C₂₋₆alkenyleneOC₁₋₆alkyl, OC₂₋₆alkynyleneNR³⁵C₂₋₆alkenyleneOC₁₋₆alkyl, OC₁₋₆alkyleneNR³⁵C₂₋₆alkynyleneOC₁₋₆alkyl, OC₂₋₆alkenyleneNR³⁵C₂₋₆alkynyleneOC₁₋₆alkyl, and OC₂₋₆alkynyleneNR³⁵C₂₋₆alkynyleneOC₁₋₆alkyl;
R³² is selected from COR³⁶, CO₂R³⁶, CONR³⁶R³⁷, OCONR³⁶R³⁷, NHSO₂R³⁶, SO₂NR³⁶R³⁷, NHCOR³⁶ and NHCO₂R³⁶;
R³³ and R³⁴ are joined to form, together with the nitrogen therebetween, a 3-6 membered saturated or unsaturated ring, optionally containing one additional heteroatom selected from NR³⁸, O, S, SO and SO₂, and substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl) or C₁₋₆alkyleneOC₁₋₆alkyl; or
R³³ and R³⁴ are joined to form, together with the nitrogen therebetween, a 7-10 membered saturated or unsaturated ring, optionally containing one additional heteroatom selected from NR³⁸, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R³⁵, R³⁶, and R³⁷ are independently selected from H and C₁₋₆alkyl;
R³⁸ is selected from D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
and
each alkyl, alkenyl, alkynyl, alkylene, alkenylene and alkynylene group is optionally fluoro-substituted.

In some embodiments, one or more compounds of the application are coupled to an antibody to form an antibody-drug conjugate. In some embodiments, the antibody targets an antigen that is specific to a type of cancer or tumor.

The present application also includes a composition comprising one or more compounds of the application and a carrier. In an embodiment, the composition is a pharmaceutical composition comprising one or more compounds of the application and a pharmaceutically acceptable carrier.

The compounds of the application have been shown to be capable of activating STING protein function. Therefore the compounds of the application are useful for treating diseases, disorders or conditions treatable by activation of STING. Accordingly, the present application also includes a method of treating a disease, disorder or condition treatable by activation of STING, comprising administering a therapeutically effective amount of one or more compounds of the application to a subject in need thereof.

In a further embodiment, the compounds of the application are used as medicaments. Accordingly, the application also includes a compound of the application for use as a medicament.

The present application also includes a use of one or more compounds of the application for treatment of a disease, disorder or condition treatable by activation of STING as well as a use of one or more compounds of the application for the preparation of a medicament for treatment of a disease, disorder or condition treatable by activation of STING. The application further includes one or more compounds of the application for use in treating a disease, disorder or condition treatable by activation of STING.

The compounds of the application are useful for treating diseases, disorders or conditions mediated by STING protein activation. Accordingly, the present application also includes a method of treating a disease, disorder or condition mediated by STING protein activation, comprising administering a therapeutically effective amount of one or more compounds of the application to a subject in need thereof.

The present application also includes a use of one or more compounds of the application for treatment of a disease, disorder or condition mediated by STING protein activation as well as a use of one or more compounds of the application for the preparation of a medicament for treatment of a disease, disorder or condition mediated by STING protein activation. The application further includes one or more compounds of the application for use in treating a disease, disorder or condition mediated by STING protein activation.

In an embodiment, the disease, disorder or condition mediated by STING protein activation, or treatable by activation of STING, is a neoplastic disorder. In an embodiment, the treatment comprises administration or use of an amount of one or compounds of the application that is effective to ameliorate at least one symptom of the neoplastic disorder, for example, reduced cell proliferation or reduced tumor mass in a subject in need of such treatment.

In an embodiment, the disease, disorder or condition mediated by STING protein activation, or treatable by activation of STING, is cancer.

In an embodiment, the disease, disorder or condition mediated by STING protein activation, or treatable by activation of STING, is a disease, disorder or condition associated with an uncontrolled and/or abnormal cellular activity affected directly or indirectly by STING. In another embodiment, the uncontrolled and/or abnormal cellular activity that is affected directly or indirectly by STING is proliferative activity in a cell.

The application also includes a method of inhibiting proliferative activity in a cell, comprising administering an effective amount of one or more compounds of the application to the cell.

In a further embodiment the disease, disorder or condition mediated by STING protein activation, or treatable by activation of STING, is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as other tyrosine-kinase inhibitors, immunotherapy, hormonal therapy and anti-angiogenic therapies.

The application additionally provides a process for the preparation of compounds of Formula I. General and specific processes are discussed in more detail and set forth in the Examples below.

Other features and advantages of the present application will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

### BRIEF DESCRIPTION OF THE DRAWINGS

The application will be described in greater detail herein below with reference to the drawings in which:

FIG. 1 shows the structure of prior art STING agonists, DMXAA and ADU-S100.

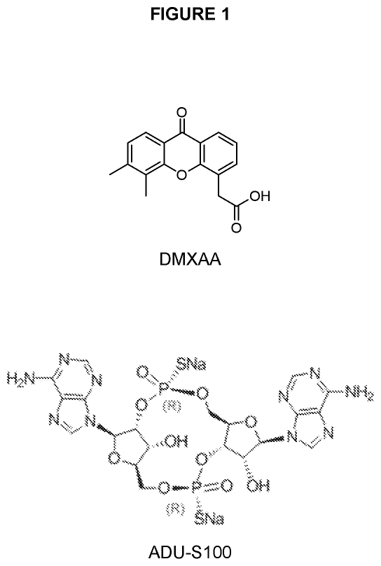

FIG. 2 shows in vivo efficacy of exemplary compound B in a murine A20 model of B cell lymphoma. Exemplary compound B exhibited significant tumoricidal properties at the injected tumor with complete tumor regression being observed in 80% of animals receiving 25 μg of exemplary compound B.

FIG. 3 shows tumor free mice from a group treated with 25 μg exemplary compound B that were re-challenged with A20 cells. Tumor re-growth was monitored for 30 days. Naïve Balb/c mice were inoculated with A20 cells as control.

### DETAILED DESCRIPTION

I. Definitions

Unless otherwise indicated, the definitions and embodiments described in this and other sections are intended to be applicable to all embodiments and aspects of the application herein described for which they are suitable as would be understood by a person skilled in the art.

Unless otherwise specified within this application or unless a person skilled in the art would understand otherwise, the nomenclature used in this application generally follows the examples and rules stated in “Nomenclature of Organic Chemistry” (Pergamon Press, 1979), Sections A, B, C, D, E, F, and H. Optionally, a name of a compound may be generated using a chemical naming program: ChemDraw, PerkinElmer or BIOVIA Draw 2016, Dassault Systemes.

The term “compound of the application” or “compound of the present application” and the like as used herein refers to a compound of Formula I or a compound of Formula II, and pharmaceutically acceptable salts, solvates and/or prodrugs thereof.

The term “composition of the application” or “composition of the present application” and the like as used herein refers to a composition comprising one or more compounds the application and at least one additional ingredient.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “suitable” as used herein means that the selection of the particular compound or conditions would depend on the specific synthetic manipulation to be performed, and the identity of the species to be transformed, but the selection would be well within the skill of a person trained in the art. All method steps described herein are to be conducted under conditions sufficient to provide the desired product. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having alternate stereochemistry. It is intended that any optical isomers, as separated, pure or partially purified optical isomers or racemic or other mixtures thereof are included within the scope of the present application.

The compounds of the present application may also exist in different tautomeric forms and it is intended that any tautomeric forms which the compounds form are included within the scope of the present application.

The compounds of the present application may further exist in varying polymorphic forms and it is contemplated that any polymorphs which form are included within the scope of the present application.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₆alkyl means an alkyl group having 1, 2, 3, 4, 5 or 6 carbon atoms. All alkyl groups are optionally fluorosubstituted unless otherwise stated.

The term “alkenyl” as used herein, whether it is used alone or as part of another group, means straight or branched chain, unsaturated alkyl groups containing at least one double bond. The number of carbon atoms that are possible in the referenced alkyl group are indicated by the prefix “Cₙ₁₋ₙ₂”. For example, the term C₂₋₆alkenyl means an alkenyl group having 2, 3, 4, 5 or 6 carbon atoms.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “alkylene” as used herein, whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms. All alkylene groups are optionally fluorosubstituted unless otherwise stated.

The term “cycloalkyl,” as used herein, whether it is used alone or as part of another group, means a saturated carbocyclic group containing one or more rings.

The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring. In an embodiment of the application, the aryl group contains from 6, 9 or 10 carbon atoms, such as phenyl, indanyl or naphthyl.

The term “heterocycloalkyl” as used herein, whether it is used alone or as part of another group, refers to cyclic groups containing at least one non-aromatic ring in which one or more of the atoms are a heteroatom selected from O, S and N. Heterocycloalkyl groups are either saturated or unsaturated (i.e. contain one or more double bonds). When a heterocycloalkyl group contains the prefix “n1-n2-membered” or “n1 or n2-membered” this prefix indicates the number of atoms in the cyclic group, of which one or more, are a heteroatom as defined above.

The term “heteroaromatic” or “heteroaryl” as used herein, whether it is used alone or as part of another group, refers to cyclic groups containing at least one heteroaromatic ring in which one or more of the atoms are a heteroatom selected from O, S and N. When a heteroaryl group contains the prefix “n1-n2-membered” or “n1 or n2-membered” this prefix indicates the number of atoms in the cyclic group, of which one or more are a heteroatom as defined above.

The term “heteroatom” as used herein, unless otherwise specified, refers to an atom other than carbon or hydrogen. Heteroatoms, such as N, may be substituted with additional substituents or hydrogen to fulfill valency requirements as would be known to those skilled in the art.

All cyclic groups contain one or more than one ring (i.e. are polycyclic). When a cyclic group contains more than one ring, the rings may be fused, bridged, spirofused or linked by a bond.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “halo” as used herein refers to a halogen atom and includes fluoro, chloro, bromo and iodo.

The term “optionally substituted” refers to groups, structures, or molecules that are either unsubstituted or are substituted with one or more substituents.

The term “fluorosubstituted” refers to the substitution of one or more, including all, hydrogens in a referenced group with fluorine.

The term “deutero-substituted” refers to the substitution of one or more, including all, hydrogens in a referenced group with deuterium.

The symbol “ [symbol] ” is used herein to represent the point of attachment of a group to the remainder of a molecule or chemical formula.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “cell” as used herein refers to a single cell or a plurality of cells and includes a cell either in a cell culture or in a subject.

The term “subject” as used herein includes all members of the animal kingdom including mammals, and suitably refers to humans. Thus the methods and uses of the present application are applicable to both human therapy and veterinary applications.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. Either the mono- or di-acid salts can be formed, and such salts can exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. [See, for example, S. M. Berge, et al., “Pharmaceutical Salts,” J. Pharm. Sci. 1977, 66, 1-19]. The selection of the appropriate salt may be useful so that an ester functionality, if any, elsewhere in a compound is not hydrolyzed. The selection criteria for the appropriate salt will be known to one skilled in the art.

Prodrugs of the compounds of the present application may be, for example, conventional esters formed with available hydroxy, thiol, amino or carboxyl groups. Some common esters which have been utilized as prodrugs are phenyl esters, aliphatic (C₁-C₂₄) esters, acyloxymethyl esters, carbamates and amino acid esters.

The term “solvate” as used herein means a compound, or a salt or prodrug of a compound, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

The term “inert organic solvent” as used herein refers to a solvent that is generally considered as non-reactive with the functional groups that are present in the compounds to be combined together in any given reaction, or in the compounds to be solubilized, so that it does not interfere with or inhibit the desired synthetic transformation. Organic solvents are typically non-polar and dissolve compounds that are not soluble in aqueous solutions.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations. For example, in some embodiments, the compounds of the application may be administered at least once a week. In some embodiments, the compounds may be administered to the subject from about one time per three weeks, or about one time per week to about once daily for a given treatment. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration may be required. For example, the compounds are administered to the subject in an amount and for duration sufficient to treat the patient.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition mediated by STING protein activation or treatable by activation of STING, or manifesting a symptom associated with a disease, disorder or condition mediated by STING protein activation or treatable by activation of STING.

The “disease, disorder or condition mediated by STING” as used herein refers to a disease, disorder or condition treatable by activation of STING activity and particularly using an STING agonist, such as one or more compounds of the application herein described.

The term “mediated by STING” as used herein means that the disease, disorder or condition to be treated is affected by, modulated by and/or has some biological basis, either direct or indirect, that includes aberrant STING activity, in particular, decreased STING activity such as results from mutation or splice variation and the like. These diseases respond favourably when STING activity associated with the disease is activated by one or more of the compounds of the application.

The term “STING agonist” as used herein refers to compounds that are “activators of STING” (i.e. activate STING activity) and therefore the term “activator” and “agonist” may be used interchangeably in reference to the compounds of the application.

As used herein, the term “effective amount” or “therapeutically effective amount” means an amount of one or more compounds of the application that is effective, at dosages and for periods of time necessary to achieve the desired result. For example in the context of treating a disease, disorder or condition mediated by STING protein activation or treatable by activation of STING, an effective amount is an amount that, for example, increases STING protein activation, or increases STING activity, compared to the activity without administration of the one or more compounds. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. In an embodiment, the effective amount is one that following treatment therewith manifests as an improvement in or reduction of any disease symptom. When the disease is cancer, amounts that are effective can cause a reduction in, for example, the number, growth rate, size and/or distribution of tumours.

The term “administered” as used herein means administration of a therapeutically effective amount of one or more compounds, or compositions, of the application to a cell either in cell culture or in a subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder. Neoplasms can be benign (such as uterine fibroids and melanocytic nevi), potentially malignant (such as carcinoma in situ) or malignant (i.e. cancer). Exemplary neoplastic disorders include but are not limited to carcinoma, sarcoma, metastatic disorders (e.g., tumors arising from the prostate), hematopoietic neoplastic disorders, (e.g., leukemias, lymphomas, myeloma and other malignant plasma cell disorders), metastatic tumors and other cancers. Prevalent cancers include breast, prostate, colon, lung, liver, brain, ovarian and pancreatic cancers.

The term “cancer” as used herein refers to cellular-proliferative disease states, including but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

II. Compounds and Compositions of the Application

Compounds of the present application were prepared and were found to inhibit uncontrolled and/or abnormal cellular activities affected directly or indirectly by activation of the STING protein. In particular, compounds of the present application exhibited activity as STING agonists, and are therefore useful in therapy, for example for the treatment of neoplastic disorders such as cancer.

Accordingly, the present application includes a compound of Formula I or a pharmaceutically acceptable salt, solvate and/or prodrug thereof:

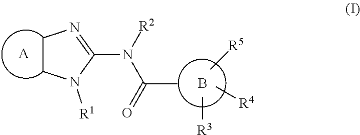

wherein:
Ring A is a 5 or 6-membered heteroaromatic ring comprising 1 to 3 heteroatoms selected from O, N and S that is substituted with at least one R⁶ and optionally substituted with one or two additional substituents independently selected from halo, C₁₋₆alkyl, C₂₋₆alkenyl, C₂₋₆alkynyl, OR^{a}, NR^{a}R^{b}, C₁₋₆alkyleneOR^{a}, C₁₋₆alkyleneNR^{a}R^{b}, OC₁₋₆alkyleneOR^{a}, OC₁₋₆alkyleneNR^{a}R^{b}, C₂₋₆alkenyleneOR^{a}, C₂₋₆alkenyleneNR^{a}R^{b}, OC₂₋₆alkenyleneOR^{a}, OC₂₋₆alkenyleneNR^{a}R^{b}, C₂₋₆alkynyleneOR^{a}, C₂₋₆alkynyleneNR^{a}R^{b}, OC₂₋₆alkynyleneOR^{a} and OC₂₋₆alkynyleneNR^{a}R^{b};
R^{a} and R^{b} are independently selected from H, C₁₋₆alkyl, C₁₋₆alkyleneOC₁₋₆alkyl, C₂₋₆alkenyleneOC₁₋₆alkyl, and C₂₋₆alkynyleneOC₁₋₆alkyl, or
R^{a} and R^{b} are joined to form, together with the atoms therebetween, a 3-10 membered saturated or unsaturated ring, optionally containing one additional heteroatom selected from NR^{s}, O S, SO and SO₂, and optionally substituted with one or more of D, OH, NH₂, CHO, CO₂H, CONH₂, C₁₋₆alkyl, C₁₋₆alkenyl, C₁₋₆alkynyl C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl; R^{s} is selected from H, D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl; R¹ is selected from C₁₋₁₀alkyleneR⁷, C₂₋₁₀alkenyleneR⁷, C₂₋₁₀alkynyleneR⁷, C₃₋₁₀cycloalkyl, 4-10-membered heterocycloalkyl, 5-10-membered heteroaryl and C₆₋₁₀aryl, the latter 4 groups and each C₁₋₁₀alkylene, C₂₋₁₀alkenylene and C₂₋₁₀alkynylene being optionally substituted with 1-4 substituents independently selected from halo, nitro, C₁₋₆alkyleneR^{c}, C₁₋₆alkyleneOR^{c}, OC₁₋₆alkyleneOR^{c}, C₁₋₆alkyleneNR^{c}R^{d}, OC₁₋₆alkyleneNR^{c}R^{d}, OR^{c}, NR^{c}R^{d}, CO₂R^{c}, CONR^{c}R^{d}, SO₂NR^{c}R^{d}, OCONR^{c}R^{d}, COR^{e}, C₃₋₁₀cycloalkyl, 3-10-membered heterocycloalkyl, 5-10-membered heteroaryl and C₆₋₁₀aryl;
R^{c} is selected from H, C₁₋₆alkyl, COC₁₋₆alkyl, C₃₋₁₀cycloalkyl, 3-10-membered heterocycloalkyl, 5-10-membered heteroaryl and C₆₋₁₀aryl;
R^{d} is selected from H and C₁₋₆alkyl;
R^{e} is selected from H, C₁₋₆alkyl, C₃₋₁₀cycloalkyl, 3-10-membered heterocycloalkyl, 5-10-membered heteroaryl and C₆₋₁₀aryl;
R² is selected from H, C₁₋₆alkyl, C₂₋₆alkenyl and C₂₋₆alkynyl;
Ring B is a 5 or 6-membered heteroaromatic ring comprising 1 to 3 heteroatoms selected from O, N and S;
R³ is selected from H, C₁₋₆alkyl, C₃₋₁₀cycloalkyl and 3-10-membered heterocycloalkyl, the latter three groups being optionally substituted with halo, OR^{f}, NR^{f}R^{g}, COR^{f}, CO₂R^{f}, CONR^{f}R^{g}, SO₂NR^{f}R^{g}, NHSO₂R^{f} and OCONR^{f}R^{g};
R^{f} and R^{g} are independently selected from H and C₁₋₆alkyl;
R⁴ is selected from H, C₁₋₆alkyl, C₂₋₆alkenyl, C₂₋₆alkynyl and C₃₋₇cycloalkyl;
R⁵ is selected from H, halo and C₁₋₆alkyl;
R⁶ is selected from COR^{h}, CO₂R^{h}, CONR^{h}Rⁱ, OCONR^{h}Rⁱ, NHSO₂R^{h}, SO₂NR^{h}Rⁱ, NHCOR^{h} and NHCO₂R^{h},
R^{h} and Rⁱ are independently selected from H and C₁₋₆alkyl;
R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₁₀cycloalkyl, 4-10-membered heterocycloalkyl, 5-10-membered heteroaryl and C₆₋₁₀aryl, the latter 4 groups being optionally substituted with 1-4 substituents independently selected from halo, nitro, C₁₋₆alkyleneR^{m}, C₁₋₆alkyleneOR^{m}, OC₁₋₆alkyleneOR^{m}, C₁₋₆alkyleneNR^{m}Rⁿ, OC₁₋₆alkyleneNR^{m}Rⁿ, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl, or R⁷ is

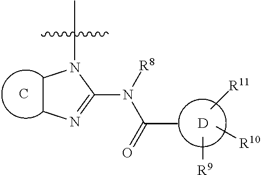

R^{j} is selected from H, C₁₋₆alkyl, COC₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, C₁₋₆alkyleneNR^{m}Rⁿ, OC₁₋₆alkyleneNR^{m}Rⁿ, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{k} is selected from H and C₁₋₆alkyl;
R^{l} is selected from H, C₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, C₁₋₆alkyleneNR^{m}Rⁿ, OC₁₋₆alkyleneNR^{m}Rⁿ, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{m} is selected from H and C₁₋₆alkyl;
Rⁿ is selected from H, C₁₋₆alkyl, C₃₋₁₀cycloalkyl, 4-10-membered heterocycloalkyl, 5-10-membered heteroaryl and C₆₋₁₀aryl;
R⁸, R⁹, R¹⁰ and R¹¹ are, independently, as defined for R², R³, R⁴ and R⁵, respectively;
Ring C is selected from phenyl and a 5 or 6-membered heteroaromatic ring comprising 1 to 3 heteroatoms selected from O, N and S, and Ring C is substituted with at least one R¹² and optionally substituted with one or two additional substituents independently selected from halo, C₁₋₆alkyl, C₂₋₆alkenyl, C₂₋₆alkynyl, OR^{o}, NR^{o}R^{p}, C₁₋₆alkyleneOR^{o}, C₁₋₆alkyleneNR^{o}R^{p}, OC₁₋₆alkyleneOR^{o}, OC₁₋₆alkyleneNR^{o}R^{p}, C₂₋₆alkenyleneOR^{o}, C₂₋₆alkenyleneNR^{o}R^{p}, OC₂₋₆alkenyleneOR^{o}, OC₂₋₆alkenyleneNR^{o}R^{p}, C₂₋₆alkynyleneOR^{o}, C₂₋₆alkynyleneNR^{o}R^{p}, OC₂₋₆alkynyleneOR^{o} and OC₂₋₆alkynyleneNR^{o}R^{p};
R^{o} and R^{p} are independently selected from H, C₁₋₆alkyl, C₁₋₆alkyleneOC₁₋₆alkyl, C₂₋₆alkenyleneOC₁₋₆alkyl, and C₂₋₆alkynyleneOC₁₋₆alkyl, or
R^{o} and R^{p} are joined to form, together with the atoms therebetween, a 3-10 membered saturated or unsaturated ring, optionally containing one additional heteroatom selected from NR^{t}, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R^{t} is selected from H, D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl; Ring D is a 5 or 6-membered heteroaromatic ring comprising 1 to 3 heteroatoms selected from O, N and S;
R¹² is selected from COR^{q}, CO₂R^{q}, CONR^{q}R^{r}, OCONR^{q}R^{r}, NHSO₂R^{q}, SO₂NR^{q}R^{r}, NHCOR^{q} and NHCO₂R^{q};
R^{q} and R^{r} are independently selected from H and C₁₋₆alkyl;
and
each alkyl, alkenyl, alkynyl, alkylene, alkenylene and alkynylene group is optionally fluoro-substituted.

The present application also includes a compound of Formula I or a pharmaceutically acceptable salt, solvate and/or prodrug thereof:

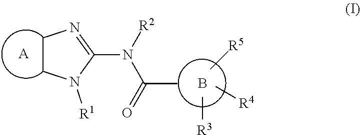

wherein:
Ring A is a 5 or 6-membered heteroaromatic ring comprising 1 to 3 heteroatoms selected from O, N and S that is substituted with at least one R⁶ and optionally substituted with one or two additional substituents independently selected from halo, C₁₋₆alkyl, C₂₋₆alkenyl, C₂₋₆alkynyl, OR^{a}, NR^{a}R^{b}, C₁₋₆alkyleneOR^{a}, OC₁₋₆alkyleneOR^{a}, C₂₋₆alkenyleneOR^{a}, OC₂₋₆alkenyleneOR^{a}, C₂₋₆alkynyleneOR^{a} and OC₂₋₆alkynyleneOR^{a};
R^{a} and R^{b} are independently selected from H and C₁₋₆alkyl;
R¹ is selected from C₁₋₁₀alkyleneR⁷, C₂₋₁₀alkenyleneR⁷, C₂₋₁₀alkynyleneR⁷, C₃₋₁₀cycloalkyl, 4-10-membered heterocycloalkyl, 5-10-membered heteroaryl and C₆₋₁₀aryl, the latter 4 groups and each C₁₋₁₀alkylene, C₂₋₁₀alkenylene and C₂₋₁₀alkynylene being optionally substituted with 1-4 substituents independently selected from halo, nitro, C₁₋₆alkyleneR^{c}, C₁₋₆alkyleneOR^{c}, OC₁₋₆alkyleneOR^{c}, OR^{c}, NR^{c}R^{d}, CO₂R^{c}, CONR^{c}R^{d}, SO₂NR^{c}R^{d}, OCONR^{c}R^{d}, COR^{e}, C₃₋₁₀cycloalkyl, 3-10-membered heterocycloalkyl, 5-10-membered heteroaryl and C₆₋₁₀aryl; R^{c} is selected from H, C₁₋₆alkyl, COC₁₋₆alkyl, C₃₋₁₀cycloalkyl, 3-10-membered heterocycloalkyl, 5-10-membered heteroaryl and C₆₋₁₀aryl;
R^{d} is selected from H and C₁₋₆alkyl;
R^{e} is selected from H, C₁₋₆alkyl, C₃₋₁₀cycloalkyl, 3-10-membered heterocycloalkyl, 5-10-membered heteroaryl and C₆₋₁₀aryl;
R² is selected from H, C₁₋₆alkyl, C₂₋₆alkenyl and C₂₋₆alkynyl;
Ring B is a 5 or 6-membered heteroaromatic ring comprising 1 to 3 heteroatoms selected from O, N and S;
R³ is selected from H, C₁₋₆alkyl, C₃₋₁₀cycloalkyl and 3-10-membered heterocycloalkyl, the latter three groups being optionally substituted with halo, OR^{f}, NR^{f}R^{g}, COR^{f}, CO₂R^{f}, CONR^{f}R^{g}, SO₂NR^{f}R^{g}, NHSO₂R^{f} and OCONR^{f}R^{g};
R^{f} and R^{g} are independently selected from H and C₁₋₆alkyl;
R⁴ is selected from H, C₁₋₆alkyl, C₂₋₆alkenyl, C₂₋₆alkynyl and C₃₋₇cycloalkyl;
R⁵ is selected from H, halo and C₁₋₆alkyl;
R⁶ is selected from COR^{h}, CO₂R^{h}, CONR^{h}Rⁱ, OCONR^{h}Rⁱ, NHSO₂R^{h}, SO₂NR^{h}Rⁱ, NHCOR^{h} and NHCO₂R^{h},
R^{h} and Rⁱ are independently selected from H and C₁₋₆alkyl;
R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₁₀cycloalkyl, 4-10-membered heterocycloalkyl, 5-10-membered heteroaryl and C₆₋₁₀aryl, the latter 4 groups being optionally substituted with 1-4 substituents independently selected from halo, nitro, C₁₋₆alkyleneR^{m}, C₁₋₆alkyleneOR^{m}, OC₁₋₆alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl, or R⁷ is

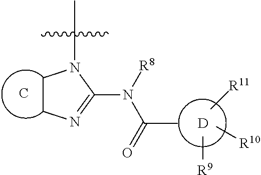

R^{j} is selected from H, C₁₋₆alkyl, COC₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{k} is selected from H and C₁₋₆alkyl;
R^{l} is selected from H, C₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{m} is selected from H and C₁₋₆alkyl;
Rⁿ is selected from H, C₁₋₆alkyl, C₃₋₁₀cycloalkyl, 4-10-membered heterocycloalkyl, 5-10-membered heteroaryl and C₆₋₁₀aryl;
R⁸, R⁹, R¹⁰ and R¹¹ are, independently, as defined for R², R³, R⁴ and R⁵, respectively;
Ring C is selected from a 5 or 6-membered heteroaromatic ring comprising 1 to 3 heteroatoms selected from O, N and S and phenyl, and is substituted with at least one R¹² and optionally substituted with one or two additional substituents independently selected from halo, C₁₋₆alkyl, C₂₋₆alkenyl, C₂₋₆alkynyl, OR^{o}, NR^{o}R^{p}, C₁₋₆alkyleneOR^{o}, OC₁₋₆alkyleneOR^{o}, C₂₋₆alkenyleneOR^{o}, OC₂₋₆alkenyleneOR^{o}, C₂₋₆alkynyleneOR^{o} and OC₂₋₆alkynyleneOR^{o};
R^{o} and R^{p} are independently selected from H and C₁₋₆alkyl;
Ring D is a 5 or 6-membered heteroaromatic ring comprising 1 to 3 heteroatoms selected from O, N and S;
R¹² is selected from COR^{a}, CO₂R^{q}, CONR^{q}R^{r}, OCONR^{q}R^{r}, NHSO₂R^{q}, SO₂NR^{q}R^{r}, NHCOR^{q} and NHCO₂R^{q};
R^{q} and R^{r} are independently selected from H and C₁₋₆alkyl; and
each alkyl, alkenyl, alkynyl, alkylene, alkenylene and alkynylene group is optionally fluoro-substituted.

In some embodiments, Ring A is selected from pyridinyl, pyrimidinyl, pyridazinyl, pyrazinyl, triazinyl, tetrazinyl, thienyl, furanyl, pyrrolyl, thiazolyl, oxazolyl and pyrazolyl. In some embodiments, Ring A is selected from pyridinyl, thienyl and furanyl. In some embodiments, Ring A is pyridinyl or thienyl.

In some embodiments, Ring A is substituted with one R⁶ and optionally substituted with one additional substituent selected from Cl, F, C₁₋₄alkyl, OR^{a}, NR^{a}R^{b}, C₁₋₄alkyleneOR^{a}, C₁₋₄alkyleneNR^{a}R^{b}, OC₁₋₄alkyleneNR^{a}R^{b} and OC₁₋₄alkyleneOR^{a}; and R^{a} and R^{b} are independently selected from H and C₁₋₄alkyl, or

R^{a} and R^{b} are joined to form, together with the atoms therebetween, a 5 or 6 membered saturated or unsaturated ring, optionally containing one additional heteroatom selected from NR^{s}, O, S, SO and SO₂.

In some embodiments, Ring A is substituted with one R⁶ and optionally substituted with one additional substituent selected from Cl, F, C₁₋₄alkyl, OR^{a}, NR^{a}R^{b}, C₁₋₄alkyleneOR^{a}, and OC₁₋₄alkyleneOR^{a}; and R^{a} and R^{b} are independently selected from H and C₁₋₄alkyl.

In some embodiments, R⁶ is selected from COR^{h}, CO₂R^{h} and CONR^{h}Rⁱ; and R^{h} and Rⁱ are independently selected from H and C₁₋₄alkyl. In some embodiments, R⁶ is selected from COR^{h}, CO₂R^{h} and CONR^{h}Rⁱ; and R^{h} and Rⁱ are independently selected from H and CH₃. In some embodiments, R⁶ is CONR^{h}Rⁱ; and R^{h} and Rⁱ are independently selected from H and CH₃. In an embodiment, R⁶ is CONH₂.

In some embodiments, Ring A is optionally substituted with one additional substituent selected from Cl, F, C₁₋₄alkyl, C₂₋₄alkenyl, C₂₋₄alkynyl, OR^{a}, NR^{a}R^{b}, C₁₋₄alkyleneOR^{a}, C₁₋₄alkyleneNR^{a}R^{b}, OC₁₋₄alkyleneOR^{a}, OC₁₋₄alkyleneNR^{a}R^{b}, C₂₋₄alkenyleneOR^{a}, C₂₋₄alkenyleneNR^{a}R^{b}, OC₂₋₄alkenyleneOR^{a}, OC₂₋₄alkenyleneNR^{a}R^{b}, C₂₋₄alkynyleneOR^{a}, C₂₋₄alkynyleneNR^{a}R^{b}, OC₂₋₄alkynyleneOR^{a} and OC₂₋₄alkyleneNR^{a}R^{b}; and R^{a} and R^{b} are independently selected from H and C₁₋₄alkyl, or R^{a} and R^{b} are joined to form, together with the atoms therebetween, a 5 or 6 membered saturated or unsaturated ring, optionally containing one additional heteroatom selected from NR^{s}, O S, SO and SO₂, and each alkyl, alkenyl, alkynyl, alkylene, alkenylene and alkynylene group is optionally fluoro-substituted.

In some embodiments, Ring A is optionally substituted with one additional substituent selected from Cl, F, C₁₋₄alkyl, C₂₋₄alkenyl, C₂₋₄alkynyl, OR^{a}, NR^{a}R^{b}, C₁₋₄alkyleneOR^{a}, C₁₋₄alkyleneNR^{a}R^{b}, OC₁₋₄alkyleneOR^{a}, OC₁₋₄alkyleneNR^{a}R^{b}, C₂₋₄alkenyleneOR^{a}, C₂₋₄alkenyleneNR^{a}R^{b}, OC₂₋₄alkenyleneOR^{a}, OC₂₋₄alkenyleneNR^{a}R^{b}, C₂₋₄alkynyleneOR^{a}, C₂₋₄alkynyleneNR^{a}R^{b}, OC₂₋₄alkynyleneOR^{a} and OC₂₋₄alkyleneNR^{a}R^{b}; and

R^{a} and R^{b} are independently selected from H, C₁₋₆alkyl, C₁₋₆alkyleneOC₁₋₆alkyl, C₂₋₆alkenyleneOC₁₋₆alkyl, and C₂₋₆alkynyleneOC₁₋₆alkyl, or
R^{a} and R^{b} are joined to form, together with the atoms therebetween, a 3-10 membered saturated or unsaturated ring, optionally containing one additional heteroatom selected from NR^{s}, O S, SO and SO₂, and optionally substituted with one or more of D, OH, NH₂, CHO, CO₂H, CONH₂, C₁₋₆alkyl, C(O)C₁₋₆alkyl, C₁₋₆alkenyl, C₁₋₆alkynyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl, and each alkyl, alkenyl, alkynyl, alkylene, alkenylene and alkynylene group is optionally fluoro-substituted.

In some embodiments, Ring A is optionally substituted with one additional substituent selected from Cl, F, CH₃, CH₂CH₃, CF₂H, OCH₃, OCH₂CH₃, OH, CF₃, OCF₃, CF₂H, OCF₂H, NH₂, NHCH₃, CH₂OCH₃, CH₂NHCH₃, CH₂NH₂, CH₂OCF₃, CH₂OCF₂H, CH₂CH₂OCH₃, CH₂CH₂NHCH₃, CH₂CH₂NH₂, CH₂CH₂OCF₂H, CH₂CH₂OCF₃, OCH₂OCH₃, OCH₂NHCH₃, OCH₂NH₂, OCH₂OCF₃, OCH₂OCF₂H, OCH₂CH₂OCH₃, OCH₂CH₂NHCH₃, OCH₂CH₂NH₂, OCH₂CH₂CH₂NHCH₃, OCH₂CH₂OCF₂H and OCH₂CH₂OCF₃. In some embodiments, Ring A is optionally substituted with one additional substituent selected from Cl, F, CH₃, CH₂CH₃, CF₂H, OCH₃, OCH₂CH₃, OH, CF₃, OCF₃, CF₂H, OCF₂H, NH₂, NHCH₃, CH₂OCH₃, CH₂OCF₃, CH₂OCF₂H, CH₂CH₂OCH₃, CH₂CH₂OCF₂H, CH₂CH₂OCF₃, OCH₂OCH₃, OCH₂OCF₃, OCH₂OCF₂H, OCH₂CH₂OCH₃, OCH₂CH₂OCF₂H and OCH₂CH₂OCF₃. In some embodiments, Ring A is substituted with one additional substituent selected from F, CH₃, CH₂CH₃, CF₂H, OCH₃, OCH₂CH₃, OH, CF₃, OCF₃, CF₂H, OCF₂H, NH₂, NHCH₃, CH₂OCH₃, CH₂NHCH₃, CH₂NH₂, CH₂OCF₃, CH₂OCF₂H, CH₂CH₂OCH₃, CH₂CH₂NHCH₃, CH₂CH₂NH₂, CH₂CH₂OCF₂H, CH₂CH₂OCF₃, OCH₂OCH₃, OCH₂NHCH₃, OCH₂NH₂, OCH₂OCF₃, OCH₂OCF₂H, OCH₂CH₂OCH₃, OCH₂CH₂NHCH₃, OCH₂CH₂NH₂, OCH₂CH₂CH₂NHCH₃, OCH₂CH₂OCF₂H and OCH₂CH₂OCF₃. In some embodiments, Ring A is substituted with one additional substituent selected from F, CH₃, CF₃, OCF₃, OCF₂H, OCH₂CH₂OCF₂H, OCH₂CH₂OCF₃, OCH₂CH₂CH₂NHCH₃, OCH₂CH₂NHCH₃ and OCH₂CH₂NH₂. In some embodiments, Ring A is substituted with one additional substituent selected from F, CH₃, CF₃, OCF₃, OCF₂H, OCH₂CH₂OCF₂H and OCH₂CH₂OCF₃.

In some embodiments, Ring A is substituted with one additional substituent selected from C₁₋₄alkyleneNR^{a}R^{b} and OC₁₋₄alkyleneNR^{a}R^{b}, wherein R^{a} and R^{b} are independently selected from H and CH₃, and each alkyl and alkylene group is optionally fluoro-substituted. In some embodiments, Ring A is substituted with one additional substituent selected from C₂₋₄alkyleneNR^{a}R^{b} and OC₂₋₄alkyleneR^{a}R^{b}, wherein R^{a} and R^{b} are independently selected from H and CH₃. In some embodiments, Ring A is substituted with one additional substituent selected from OCH₂CH₂CH₂NH₂, OCH₂CH₂CH₂N(CH₃)₂ and OCH₂CH₂CH₂NHCH₃.

In some embodiments, Ring A is substituted with one additional substituent selected from C₁₋₄alkyleneNR^{a}R^{b} and OC₁₋₄alkyleneNR^{a}R^{b}, wherein R^{a} and R^{b} are independently selected from H, C₁₋₆alkyl, C₁₋₆alkenyl, C₁₋₆alkynyl, C₁₋₆alkyleneOC₁₋₆alkyl, C₂₋₆alkenyleneOC₁₋₆alkyl, and C₂₋₆alkynyleneOC₁₋₆alkyl, and each alkyl, alkenyl, alkynyl, alkylene, alkenylene and alkynylene group is optionally fluoro-substituted. In some embodiments, Ring A is substituted with one additional substituent selected from C₁₋₄alkyleneNR^{a}R^{b} and OC₁₋₄alkyleneR^{a}R^{b}, wherein R^{a} is selected from H and C₁₋₆alkyl and R^{b} is selected from C₁₋₆alkyleneOC₁₋₆alkyl, C₂₋₆alkenyleneOC₁₋₆alkyl, and C₂₋₆alkynyleneOC₁₋₆alkyl, and each alkyl, alkylene, alkenylene and alkynylene group is optionally fluoro-substituted. In some embodiments, Ring A is substituted with one additional substituent selected from C₁₋₄alkyleneNR^{a}R^{b} and OC₁₋₄alkyleneR^{a}R^{b}, wherein R^{a} is selected from H and C₁₋₆alkyl and R^{b} is C₁₋₆alkyleneOC₁₋₆alkyl, and each alkyl and alkylene group is optionally fluoro-substituted. In some embodiments, Ring A is substituted with OC₁₋₄alkyleneR^{a}R^{b}, wherein R^{a} is selected from H and C₁₋₆alkyl and R^{b} is selected from C₁₋₆alkyleneOC₁₋₆alkyl, C₂₋₆alkenyleneOC₁₋₆alkyl, C₁₋₆alkynyleneOC₁₋₆alkyl, C₁₋₆alkyleneOC₁₋₆fluoroalkyl, C₂₋₆alkenyleneOC₁₋₆fluoroalkyl, and C₁₋₆alkynyleneOC₁₋₆fluoroalkyl. In some embodiments, Ring A is substituted with OC₁₋₄alkyleneR^{a}R^{b}, wherein R^{a} is selected from H and C₁₋₆alkyl and R^{b} is C₁₋₆alkyleneOC₁₋₆alkyl or C₁₋₆alkyleneOC₁₋₆fluoroalkyl. In some embodiments, Ring A is substituted OC₁₋₄alkyleneR^{a}R^{b}, wherein R^{a} is selected from H and C₁₋₄alkyl and R^{b} is C₁₋₄alkyleneOC₁₋₄alkyl or C₁₋₄alkyleneOC₁₋₄fluoroalkyl. In some embodiments, R^{b} is selected from CH₂OCH₃, CH₂OCF₃, CH₂OCF₂H, CH₂CH₂OCH₃, CH₂CH₂OCF₂H and CH₂CH₂OCF₃. In some embodiments, R^{b} is selected from CH₂CH₂OCH₃, CH₂CH₂OCF₂H and CH₂CH₂OCF₃. In some embodiments, R^{b} is selected from CH₂CH₂OCH₃ and CH₂CH₂OCF₂H.

In some embodiments, Ring A is substituted with one additional substituent selected from C₁₋₄alkyleneNR^{a}R^{b} and OC₁₋₄alkyleneNR^{a}R^{b}, wherein R^{a} and R^{b} are joined to form, together with the atoms therebetween, a 3-6 membered saturated or unsaturated ring, optionally containing one additional heteroatom selected from NR^{s}, O S, SO and SO₂, and each alkylene group is optionally fluoro-substituted. In some embodiments, Ring A is substituted with one additional substituent selected from C₁₋₄alkyleneNR^{a}R^{b} and OC₁₋₄alkyleneNR^{a}R^{b}, wherein R^{a} and R^{b} are joined to form, together with the atoms therebetween, a 3 or 4 membered saturated or unsaturated ring, optionally containing one additional heteroatom selected from NR^{s}, O and S and each alkylene group is optionally fluoro-substituted. In some embodiments, R^{a} and R^{b} are joined to form, together with the atoms therebetween, aziranyl, azetidinyl or diazetidinyl. In some embodiments, Ring A is substituted with one additional substituent selected from C₁₋₄alkyleneNR^{a}R^{b} and OC₁₋₄alkyleneNR^{a}R^{b}, wherein R^{a} and R^{b} are joined to form, together with the atoms therebetween, a 5 or 6 membered saturated or unsaturated ring, optionally containing one additional heteroatom selected from NR^{s}, O S, SO and SO₂, and each alkylene group is optionally fluoro-substituted. In some embodiments, R^{a} and R^{b} are joined to form, together with the atoms therebetween, pyrrolidinyl, imidazolidinyl, pyrazolidinyl, thiazolidinyl, isothiazolidinyl, piperidinyl, piperazinyl, thiomorpholinyl, or morpholinyl. In some embodiments, R^{a} and R^{b} are joined to form, together with the atoms therebetween, piperidinyl, piperazinyl, or morpholinyl. In some embodiments, R^{a} and R^{b} are joined to form, together with the atoms therebetween, piperazinyl or morpholinyl. In some embodiments, R^{a} and R^{b} are joined to form, together with the atoms therebetween, pyrrolidinyl.

In some embodiments, Ring A is substituted with one additional substituent selected from C₁₋₄alkyleneNR^{a}R^{b} and OC₁₋₄alkyleneNR^{a}R^{b}, and R^{a} and R^{b} are joined to form, together with the atoms therebetween, a 7-10 membered saturated or unsaturated ring, optionally containing one additional heteroatom selected from NR^{s}, O, S, SO and SO₂, and each alkylene group is optionally fluoro-substituted. In some embodiments, R^{a} and R^{b} are joined to form, together with the atoms therebetween, azepanyl, axepinyl, diazepinyl, oxazepanyl, thiazepinyl, azocanyl, azocinyl, azonanyl or azocinyl. In some embodiments, R^{a} and R^{b} are joined to form, together with the atoms therebetween, azepanyl, axepinyl, diazepinyl, or oxazepanyl. In some embodiments, R^{a} and R^{b} are joined to form, together with the atoms therebetween, oxazepanyl.

In some embodiments, R^{s} is selected from H, D, CHO, CO₂H, CONH₂, C₁₋₄alkyl, C₁₋₄alkenyl, C₁₋₄alkynyl, C(O)C₁₋₆alkyl, CO₂C₁₋₄alkyl, OC₁₋₄alkyl, CONHC₁₋₄alkyl, CON(C₁₋₄alkyl)(C₁₋₄alkyl), C₁₋₄alkyleneOC₁₋₄alkyl, C₂₋₄alkenyleneOC₁₋₄alkyl, and C₂₋₄alkynyleneOC₁₋₄alkyl, and each alkyl, alkenyl, alkynyl, alkylene, alkenylene and alkynylene group is optionally fluoro-substituted. In some embodiments. In some embodiments, R^{s} is selected from H, D, C₁₋₄alkyl, C(O)C₁₋₄alkyl, CO₂C₁₋₄alkyl, CONHC₁₋₄alkyl, C₁₋₄alkyleneOC₁₋₄alkyl, and each alkyl, alkylene and alkenylene group is optionally fluoro-substituted. In some embodiments, R^{s} is selected from H, D, C₁₋₄alkyl, and C(O)C₁₋₄alkyl, and each alkyl group is optionally fluoro-substituted. In some embodiments, R^{s} is selected from H, D, CH₃, CF₃, CHF₂, C(O)CH₃, C(O)CF₃, and C(O)CHF₂. In some embodiments, R^{s} is selected from H, D, CH₃, CF₃, and CHF₂. In some embodiments, R^{s} is selected from C(O)CH₃, C(O)CF₃, and C(O)CHF₂. In some embodiments, R^{s} is C(O)CH₃.

In some embodiments, R^{a} and R^{b} are joined to form, together with the atoms therebetween, a 3-6 membered saturated or unsaturated ring or a 7-10-membered saturated or unsaturated ring, and the 3-6 membered saturated or unsaturated ring or the 7-10-membered saturated or unsaturated ring optionally contain one additional heteroatom selected from NR^{s}, O, S, SO and SO₂, and are optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl, and each alkyl, alkenyl, alkynyl, alkylene, alkenylene and alkynylene group is optionally fluoro-substituted. In some embodiments, R^{a} and R^{b} are joined to form, together with the atoms therebetween, a 3-6 membered saturated or unsaturated ring or a 7-10-membered saturated or unsaturated ring, and the 3-6 membered saturated or unsaturated ring or the 7-10-membered saturated or unsaturated ring optionally contain one additional heteroatom selected from NR^{s}, O, S, SO and SO₂, and are optionally substituted with one or more of D, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, C₁₋₆alkyleneOC₁₋₆alkyl, C₂₋₆alkenyleneOC₁₋₆alkyl, and C₂₋₆alkynyleneOC₁₋₆alkyl, and each alkyl, alkenyl, alkynyl, alkylene, alkenylene and alkynylene group is optionally fluoro-substituted. In some embodiments, R^{a} and R^{b} are joined to form, together with the atoms therebetween, a 3-6 membered saturated or unsaturated ring or a 7-10-membered saturated or unsaturated ring, the 3-6 membered saturated or unsaturated ring or the 7-10-membered saturated or unsaturated ring optionally contain one additional heteroatom selected from NR^{s}, O, S, SO and SO₂, and are optionally substituted with one or more of D, C₁₋₆alkyl, C(O)C₁₋₆alkyl, OC₁₋₆alkyl, or C₁₋₆alkyleneOC₁₋₆alkyl, and each alkyl and alkenylene group is optionally fluoro-substituted.

In some embodiments, R^{a} and R^{b} are joined to form, together with the atoms therebetween, a 5 or 6 membered saturated or unsaturated ring, optionally containing one additional heteroatom selected from NR^{s}, O, S, SO and SO₂, and substituted with one or more of D. In some embodiments, R^{a} and R^{b} are joined to form, together with the atoms therebetween, a 5 or 6 membered saturated or unsaturated ring that is deutero-substituted piperidinyl, piperazinyl, or morpholinyl. In some embodiments, R^{a} and R^{b} are joined to form, together with the atoms therebetween, a deutero-substituted morpholinyl. In some embodiments, R^{a} and R^{b} are joined to form, together with the atoms therebetween, a 5 or 6 membered saturated or unsaturated ring that is substituted with C(O)C₁₋₆alkyl or C(O)C₁₋₆fluoroalkyl, suitably C(O)C₁₋₄alkyl or C(O)C₁₋₄fluoroalkyl, more suitably C(O)CH₃, C(O)CHF₂, or C(O)CHF₃, most suitably C(O)CH₃. In some embodiments, R^{a} and R^{b} are joined to form, together with the atoms therebetween, a 5 or 6 membered saturated or unsaturated ring, optionally containing one additional heteroatom selected from NR^{s}, O, S, SO and SO₂, and substituted with C₁₋₆alkyleneOC₁₋₆alkyl that is optionally fluoro-substituted, suitably C₁₋₄alkyleneOC₁₋₄alkyl that is optionally fluoro-substituted, more suitably CH₂OCH₃, CH₂OCF₃, CH₂OCF₂H, CH₂CH₂OCH₃, CH₂CH₂OCF₂H and CH₂CH₂OCF₃, most suitably CH₂OCH₃, CH₂OCF₃, and CH₂OCF₂H.

In some embodiments, R^{a} and R^{b} are joined to form, together with the atoms therebetween, a 3-10 membered saturated or unsaturated ring, containing one additional heteroatom selected from NR^{s}, O, and S. In some embodiments, R^{a} and R^{b} are joined to form, together with the atoms therebetween, a 3-10 membered saturated or unsaturated ring, containing one additional heteroatom selected from S, SO and SO₂. In some embodiments, R^{a} and R^{b} are joined to form, together with the atoms therebetween, a 3-10 membered saturated or unsaturated ring, containing one additional heteroatom selected from NR^{s} and O. In an embodiment, Ring A has no additional substituent.

In some embodiments, Ring A is pyridine and the compound of Formula I has one or the following structures:

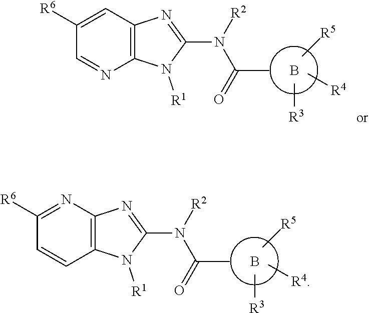

In some embodiments, the compound of Formula I has the following structure:

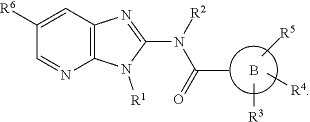

In some embodiments, Ring A is thienyl and the compound of Formula I has one of the following structures:

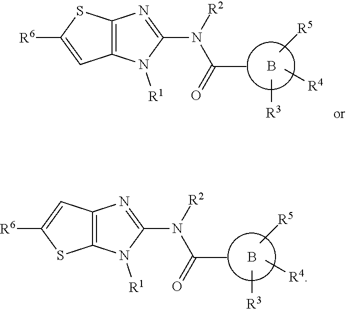

In some embodiments, the compound of Formula I has the following structure:

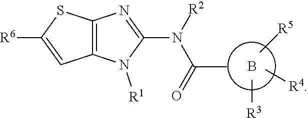

In some embodiments, R¹ is selected from C₁₋₆alkyleneR⁷, C₂₋₆alkenyleneR⁷, C₂₋₆alkynyleneR⁷, C₃₋₁₀cycloalkyl, 4-10-membered heterocycloalkyl, 5-10-membered heteroaryl and C₆₋₁₀aryl, the latter 4 groups and each C₁₋₆alkylene, C₂₋₆alkenylene and C₂₋₆alkynylene being optionally substituted with 1-2 substituents independently selected from Cl, F, C₁₋₄alkyleneR^{c}, C₁₋₄alkyleneOR^{c}, OC₁₋₄alkyleneOR^{c}, OR^{c}, NR^{c}R^{d}, CO₂R^{c}, CONR^{c}R^{d}, SO₂NR^{c}R^{d}, OCONR^{c}R^{d}, COR^{e}, C₃₋₆cycloalkyl, 3-6-membered heterocycloalkyl, 5-6-membered heteroaryl and phenyl;

R^{c} is selected from H, C₁₋₄alkyl, COC₁₋₄alkyl, C₃₋₆cycloalkyl, 3-6-membered heterocycloalkyl, 5-6-membered heteroaryl and phenyl;
R^{d} is selected from H and C₁₋₄alkyl;
R^{e} is selected from H, C₁₋₄alkyl, C₃₋₆cycloalkyl, 3-6-membered heterocycloalkyl, 5-6-membered heteroaryl and phenyl, and each alkyl, alkenyl, alkynyl, alkylene, alkenylene and alkynylene group is optionally fluoro-substituted.

In some embodiments, R¹ is selected from C₁₋₆alkyleneR⁷, C₂₋₆alkenyleneR⁷ and C₂₋₆alkynyleneR⁷; and each C₁₋₆alkylene, C₂₋₆alkenylene and C₂₋₆alkynylene is optionally substituted with 1-2 substituents independently selected from F, C₁₋₄alkyleneR^{c}, C₁₋₄alkyleneOR^{c}, OC₁₋₄alkyleneOR^{c}, OR^{c}, NR^{c}R^{d}, CO₂R^{c}, CONR^{c}R^{d}, SO₂NR^{c}R^{d}, OCONR^{c}R^{d} and COR^{e}; and R^{c}, R^{d} and R^{e} are independently selected from H and C₁₋₄alkyl, and each alkyl, alkylene, alkenylene and alkynylene group is optionally fluoro-substituted. In some embodiments, R¹ is selected from C₁₋₄alkyleneR⁷, C₂₋₄alkenyleneR⁷ and C₂₋₄alkynyleneR⁷; and each C₁₋₄alkylene, C₂₋₄alkenylene and C₂₋₄alkynylene being optionally substituted with 1 substituents independently selected from F, C₁₋₄alkyl, C₁₋₄alkyleneOR^{c}, OC₁₋₄alkyleneOR^{c} and OR^{c}; and R^{c} is selected from H and C₁₋₄alkyl, and each alkyl, alkylene, alkenylene and alkynylene group is optionally fluoro-substituted.

In some embodiment, R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₁₀cycloalkyl, 4-10-membered heterocycloalkyl, 5-10-membered heteroaryl and C₆₋₁₀aryl, the latter 4 groups being optionally substituted with 1-2 substituents independently selected from Cl, F, C₁₋₆alkyleneR^{m}, C₁₋₆alkyleneOR^{m}, OC₁₋₆alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl, or R⁷ is

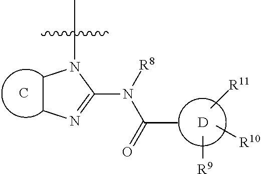

R^{j} is selected from H, C₁₋₄alkyl, COC₁₋₄alkyl, C₃₋₁₀cycloalkyl, 4-10-membered heterocycloalkyl, 5-10-membered heteroaryl and C₆₋₁₀aryl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{k} is selected from H and C₁₋₄alkyl;
R^{l} is selected from H, C₁₋₄alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{m} is selected from H and C₁₋₄alkyl;
Rⁿ is selected from H, C₁₋₄alkyl, C₃₋₁₀cycloalkyl, 4-10-membered heterocycloalkyl, 5-10-membered heteroaryl and C₆₋₁₀aryl;
R⁸, R⁹, R¹⁰ and R¹¹ are, independently, as defined for R², R³, R⁴ and R⁵, respectively, above;
Ring C is selected from phenyl and a 5 or 6-membered heteroaromatic ring comprising 1 to 3 heteroatoms selected from O, N and S, and Ring C is substituted with at least one R¹² and optionally substituted with one or two additional substituents independently selected from Cl, F, C₁₋₄alkyl, C₂₋₄alkenyl, C₂₋₄alkynyl, OR^{o}, NR^{o}R^{p}, C₁₋₄alkyleneOR^{o}, C₁₋₄alkyleneNR^{o}R^{p}, OC₁₋₄alkyleneOR^{o}, OC₁₋₄alkyleneNR^{o}R^{p}, C₂₋₄alkenyleneOR^{o}, C₂₋₄alkenyleneNR^{o}R^{p}OC₂₋₄alkenyleneOR^{o}, OC₂₋₄alkenyleneNR^{o}R^{p}, C₂₋₄alkynyleneOR^{o}, C₂₋₄alkynyleneNR^{o}R^{p}, OC₂₋₄alkynyleneOR^{o} and OC₂₋₄alkynyleneNR^{o}R^{p};
R^{o} and R^{p} are independently selected from H and C₁₋₄alkyl; or R^{o} and R^{p} are joined to form, together with the atoms therebetween, a 5 or 6 membered saturated or unsaturated ring, optionally containing one additional heteroatom selected from NR^{t}, O, S, SO and SO₂,
R^{t} is selected from H and C₁₋₄alkyl;
Ring D is a 5 or 6-membered heteroaromatic ring comprising 1 to 3 heteroatoms selected from O, N and S;
R¹² is selected from COR^{a}, CO₂R^{q}, CONR^{q}R^{r}, OCONR^{q}R^{r}, NHSO₂R^{q}, SO₂NR^{q}R^{r}, NHCOR^{q} and NHCO₂R^{q};
R^{q} and R^{r} are independently selected from H and C₁₋₄alkyl,
and each alkyl, alkenyl, alkynyl, alkylene, alkenylene and alkynylene group is optionally fluoro-substituted.

In some embodiments, R⁷ is selected from OR^{j}, NR^{j}R^{k}, NHCO₂Rⁱ, NHCOR^{l}, CO₂R^{j}, COR^{l}, CONR^{j}R^{k}, OCONR^{j}R^{k}, NHSO₂Rⁱ, SO₂NR^{j}R^{k}, C₃₋₁₀cycloalkyl, 4-10-membered heterocycloalkyl, 5-10-membered heteroaryl and C₆₋₁₀aryl, the latter 4 groups being optionally substituted with 1-2 substituents independently selected from Cl, F, C₁₋₆alkyleneR^{m}, C₁₋₆alkyleneOR^{m}, OC₁₋₆alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;

R^{j} is selected from H, C₁₋₄alkyl, COC₁₋₄alkyl, C₃₋₁₀cycloalkyl, 4-10-membered heterocycloalkyl, 5-10-membered heteroaryl and C₆₋₁₀aryl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{k} is selected from H and C₁₋₄alkyl;
R^{l} is selected from H, C₁₋₄alkyl, C₃₋₁₀cycloalkyl, 4-10-membered heterocycloalkyl, 5-10-membered heteroaryl and C₆₋₁₀aryl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{m} is selected from H and C₁₋₄alkyl;
Rⁿ is selected from H, C₁₋₄alkyl, C₃₋₁₀cycloalkyl, 4-10-membered heterocycloalkyl, 5-10-membered heteroaryl and C₆₋₁₀aryl,
and each alkyl, alkenyl, alkynyl, alkylene, alkenylene and alkynylene group is optionally fluoro-substituted.

In some embodiments, R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₆cycloalkyl, 4-6-membered heterocycloalkyl, 5-6-membered heteroaryl and phenyl, the latter 4 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₆cycloalkyl, 4-6-membered heterocycloalkyl, 5-6-membered heteroaryl and phenyl;

R^{j} is selected from H, C₁₋₄alkyl, COC₁₋₄alkyl, C₃₋₆cycloalkyl, 4-6-membered heterocycloalkyl, 5-6-membered heteroaryl and phenyl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₆cycloalkyl, 4-6-membered heterocycloalkyl, 5-6-membered heteroaryl and phenyl;
R^{k} is selected from H and C₁₋₄alkyl;
R^{l} is selected from H, C₁₋₄alkyl, C₃₋₆cycloalkyl, 4-6-membered heterocycloalkyl, 5-6-membered heteroaryl and phenyl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₆cycloalkyl, 4-6-membered heterocycloalkyl, 5-6-membered heteroaryl and phenyl;
R^{m} is selected from H and C₁₋₄alkyl;
Rⁿ is selected from H, C₁₋₄alkyl, C₃₋₁₀cycloalkyl, 4-6-membered heterocycloalkyl, 5-6-membered heteroaryl and phenyl,
and each alkyl, alkenyl, alkynyl, alkylene, alkenylene and alkynylene group is optionally fluoro-substituted.

In some embodiments, R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₆cycloalkyl, 4-6-membered heterocycloalkyl, 5-6-membered heteroaryl and phenyl, the latter 4 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ and COR^{m};

R^{j} is selected from H, C₁₋₄alkyl, COC₁₋₄alkyl, C₃₋₆cycloalkyl, 4-6-membered heterocycloalkyl, 5-6-membered heteroaryl and phenyl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ and COR^{m};
R^{k} is selected from H and C₁₋₄alkyl;
R^{l} is selected from H, C₁₋₄alkyl, C₃₋₆cycloalkyl, 4-6-membered heterocycloalkyl, 5-6-membered heteroaryl and phenyl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ and COR^{m};
R^{m} and Rⁿ are independently selected from H and C₁₋₄alkyl,
and each alkyl, alkenyl, alkynyl, alkylene, alkenylene and alkynylene group is optionally fluoro-substituted.

In some embodiments, R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₆cycloalkyl, 4-6-membered heterocycloalkyl, 5-6-membered heteroaryl and phenyl, the latter 4 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m} and phenyl;

R^{j} is selected from H, C₁₋₄alkyl, COC₁₋₄alkyl, C₃₋₆cycloalkyl, 4-6-membered heterocycloalkyl, 5-6-membered heteroaryl and phenyl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m} and phenyl;
R^{k} is selected from H and C₁₋₄alkyl;
R^{l} is selected from H, C₁₋₄alkyl, C₃₋₆cycloalkyl, 4-6-membered heterocycloalkyl, 5-6-membered heteroaryl and phenyl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m} and phenyl;
R^{m} and Rⁿ are independently selected from H and C₁₋₄alkyl,
and each alkyl, alkenyl, alkynyl, alkylene, alkenylene and alkynylene group is optionally fluoro-substituted.

In some embodiments, R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j} and SO₂NR^{j}R^{k}; and R^{j}, R^{k} and R^{l} are independently selected from H, C₁₋₄alkyl, fluoro-substituted C₁₋₄alkyl and CH₂Ph. In some embodiments, R⁷ is selected from OR^{j}, NR^{j}R^{k}, NHCO₂R^{j}, NHCOR^{l}, OCONR^{j}R^{k}, NHSO₂R^{j} and SO₂NR^{j}R^{k}; and R^{j}, R^{k} and R^{l} are independently selected from H, C₁₋₄alkyl, fluoro-substituted C₁₋₄alkyl and CH₂Ph. In some embodiments, R⁷ is selected from OR^{j}, and NHCO₂R^{j}; and R^{j}, R^{k} and R^{l} are independently selected from H, C₁₋₄alkyl and fluoro-substituted C₁₋₄alkyl. In some embodiments, R⁷ is selected from OCH₃, OCF₂H, OCF₃, NHCO₂tBu and NHCO₂CH₂Ph.

In some embodiments, R⁷ is

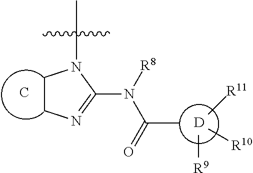

wherein R⁸, R⁹, R¹⁰ and R¹¹ are, independently, as defined for R², R³, R⁴ and R⁵, respectively, above;
Ring C is selected from phenyl and a 5 or 6-membered heteroaromatic ring comprising 1 to 3 heteroatoms selected from O, N and S, and Ring C is substituted with at least one R¹² and optionally substituted with one or two additional substituents independently selected from Cl, F, C₁₋₄alkyl, C₂₋₄alkenyl, C₂₋₄alkynyl, OR^{o}, NR^{o}R^{p}, C₁₋₄alkyleneOR^{o}, C₁₋₄alkyleneNR^{o}R^{p}, OC₁₋₄alkyleneOR^{o}, OC₁₋₄alkyleneNR^{o}R^{p}, C₂₋₄alkenyleneOR^{o}, C₂₋₄alkenyleneNR^{o}R^{p}, OC₂₋₄alkenyleneOR^{o}, OC₂₋₄alkenyleneNR^{o}R^{p}, C₂₋₄alkynyleneOR^{o}, C₂₋₄alkynyleneNR^{o}R^{p}, OC₂₋₄alkynyleneOR^{o} and OC₂₋₄alkynyleneNR^{o}R^{p};
R^{o} and R^{p} are independently selected from H and C₁₋₄alkyl, or R^{o} and R^{p} are joined to form, together with the atoms therebetween, a 5 or 6 membered saturated or unsaturated ring, optionally containing one additional heteroatom selected from NR^{t}, O, S, SO and SO₂,
R^{t} is selected from H and C₁₋₄alkyl;
Ring D is a 5 or 6 membered heteroaromatic ring comprising 1 to 3 heteroatoms selected from O, N and S;
R¹² is selected from COR^{a}, CO₂R^{q}, CONR^{q}R^{r}, OCONR^{q}R^{r}, NHSO₂R^{q}, SO₂NR^{q}R^{r}, NHCOR^{q} and NHCO₂R^{q};
R^{q} and R^{r} are independently selected from H and C₁₋₄alkyl,
and each alkyl, alkenyl, alkynyl, alkylene, alkenylene and alkynylene group is optionally fluoro-substituted.

In some embodiments, Ring C is selected from phenyl, pyridinyl, pyrimidinyl, pyridazinyl, pyrazinyl, triazinyl, tetrazinyl, thienyl, furanyl, pyrrolyl, thiazolyl, oxazolyl, and pyrazolyl. In some embodiments, Ring C is selected from phenyl, pyridinyl, thienyl and furanyl. In some embodiments, Ring C is phenyl, pyridinyl or thienyl.

In some embodiments, Ring C is substituted with one R¹² and optionally substituted with one additional substituent independently selected from Cl, F, C₁₋₄alkyl, OR^{o}, NR^{o}R^{p}, C₁₋₄alkyleneOR^{o}, NHC₁₋₄alkyleneNR^{o}R^{p}, OC₁₋₄alkyleneNR^{o}R^{p} and OC₁₋₄alkyleneOR; and R^{o} and R^{p} are independently selected from H and C₁₋₄alkyl, or R^{o} and R^{p} are joined to form, together with the atoms therebetween, a 5 or 6 membered saturated or unsaturated ring, optionally containing one additional heteroatom selected from NR^{t}, O S, SO and SO₂, and each alkyl, alkenyl, alkynyl, alkylene, alkenylene and alkynylene group is optionally fluoro-substituted.

In some embodiments, R¹² is selected from COR^{q}, CO₂R^{q} and CONR^{q}R^{r}; and R^{q} and R^{r} are independently selected from H and C₁₋₄alkyl. In some embodiments, R¹² is selected from COR^{a}, CO₂R^{q} and CONR^{q}R^{r}; and R^{q} and R^{r} are independently selected from H and CH₃. In some embodiments, R¹² is CONR^{q}R^{r}; and R^{q} and R^{r} are independently selected from H and CH₃. In an embodiment, R¹² is CONH₂.

In some embodiments, Ring C is optionally substituted with one additional substituent selected from Cl, F, C₁₋₄alkyl, C₂₋₄alkenyl, C₂-alkynyl, OR^{o}, NR^{o}R^{p}, C₁₋₄alkyleneOR^{o}, OC₁₋₄alkyleneOR^{o}, C₁₋₄alkyleneNR^{o}R^{p}, OC₁₋₄alkyleneNR^{o}R^{p}, C₂₋₄alkenyleneOR^{o}, C₂₋₄alkenyleneNR^{o}R^{p}, OC₂₋₄alkenyleneOR^{o}, OC₂₋₄alkenyleneNR^{o}R^{p}, C₂₋₄alkynyleneOR^{o}, C₂₋₄alkynyleneNR^{o}R^{p}, OC₂₋₄alkynyleneOR^{o} and OC₂₋₄alkynyleneNR^{o}R^{p}; and R^{o} and R^{p} are independently selected from H and C₁₋₄alkyl, or R^{o} and R^{p} are joined to form, together with the atoms therebetween, a 5 or 6 membered saturated or unsaturated ring, optionally containing one additional heteroatom selected from NR^{t}, O S, SO and SO₂, and each alkyl, alkenyl, alkynyl, alkylene, alkenylene and alkynylene group is optionally fluoro-substituted.

In some embodiments, Ring C is optionally substituted with one additional substituent selected from Cl, F, C₁₋₄alkyl, C₂₋₄alkenyl, C₂-alkynyl, OR^{o}, NR^{o}R^{p}, C₁₋₄alkyleneOR^{o}, OC₁₋₄alkyleneOR^{o}, C₁₋₄alkyleneNR^{o}R^{p}, OC₁₋₄alkyleneNR^{o}R^{p}, C₂₋₄alkenyleneOR^{o}, C₂₋₄alkenyleneNR^{o}R^{p}, OC₂₋₄alkenyleneOR^{o}, OC₂₋₄alkenyleneNR^{o}R^{p}, C₂₋₄alkynyleneOR^{o}, C₂₋₄alkynyleneNR^{o}R^{p}, OC₂₋₄alkynyleneOR^{o} and OC₂₋₄alkynyleneNR^{o}R^{p}; and R^{o} and R^{p} are independently selected from H and C₁₋₄alkyl, and each alkyl, alkenyl, alkynyl, alkylene, alkenylene and alkynylene group is optionally fluoro-substituted

In some embodiments, Ring C is optionally substituted with one additional substituent selected from Cl, F, CH₃, CH₂CH₃, CF₂H, OCH₃, OCH₂CH₃, OH, CF₃, OCF₃, CF₂H, OCF₂H, NH₂, NHCH₃, CH₂OCH₃, CH₂NHCH₃, CH₂NH₂, CH₂OCF₃, CH₂OCF₂H, CH₂CH₂OCH₃, CH₂CH₂NHCH₃, CH₂CH₂NH₂, CH₂CH₂OCF₂H, CH₂CH₂OCF₃, OCH₂OCH₃, OCH₂NHCH₃, OCH₂NH₂, OCH₂OCF₃, OCH₂OCF₂H, OCH₂CH₂OCH₃, OCH₂CH₂NHCH₃, OCH₂CH₂NH₂, OCH₂CH₂CH₂NHCH₃, OCH₂CH₂OCF₂H and OCH₂CH₂OCF₃. In some embodiments, Ring C is substituted with one additional substituent selected from F, CH₃, CH₂CH₃, CF₂H, OCH₃, OCH₂CH₃, OH, CF₃, OCF₃, CF₂H, OCF₂H, NH₂, NHCH₃, CH₂OCH₃, CH₂OCF₃, CH₂OCF₂H, CH₂CH₂OCH₃, CH₂CH₂OCF₂H, CH₂CH₂OCF₃, OCH₂OCH₃, OCH₂OCF₃, OCH₂OCF₂H, OCH₂CH₂OCH₃, OCH₂CH₂OCF₂H and OCH₂CH₂OCF₃. In some embodiments, Ring C is substituted with one additional substituent selected from F, CH₃, CF₃, OCF₃, OCF₂H, OCH₂CH₂OCF₂H and OCH₂CH₂OCF₃. In some embodiments, Ring C is substituted with one additional substituent selected from F, CH₃, CF₃, OCF₃, OCF₂H, OCH₂CH₂OCF₂H, and OCH₂CH₂OCF₃. In some embodiments, Ring C is substituted with one additional substituent selected from C₁₋₄alkyleneNR^{o}R^{p} and OC₁₋₄alkyleneNR^{o}R^{p}, wherein R^{o} and R^{p} are independently selected from H and CH₃, and each alkyl and alkylene, group is optionally fluoro-substituted In some embodiments, Ring C is substituted with one additional substituent selected from C₂₋₄alkyleneNR^{o}R^{p} and OC₂₋₄alkyleneR^{o}R^{p}, wherein R^{o} and R^{p} are independently selected from H and CH₃. In some embodiments, Ring C is substituted with one additional substituent selected from OCH₂CH₂CH₂NH₂, OCH₂CH₂CH₂N(CH₃)₂ and OCH₂CH₂CH₂NHCH₃.

In some embodiments, Ring C is substituted with one additional substituent selected from C₁₋₄alkyleneNR^{o}R^{p} and OC₁₋₄alkyleneNR^{o}R^{p}, wherein R^{o} and R^{p} are independently selected from H, C₁₋₆alkyl, C₁₋₆alkenyl, C₁₋₆alkynyl, C₁₋₆alkyleneOC₁₋₆alkyl, C₂₋₆alkenyleneOC₁₋₆alkyl, and C₂₋₆alkynyleneOC₁₋₆alkyl, and each alkyl, alkenyl, alkynyl, alkylene, alkenylene and alkynylene group is optionally fluoro-substituted. In some embodiments, Ring C is substituted with one additional substituent selected from C₁₋₄alkyleneNR^{o}R^{p} and OC₁₋₄alkyleneR^{o}R^{p}, wherein R^{o} is selected from H and C₁₋₆alkyl and R^{p} is selected from C₁₋₆alkyleneOC₁₋₆alkyl, C₂₋₆alkenyleneOC₁₋₆alkyl, and C₂₋₆alkynyleneOC₁₋₆alkyl, and each alkynyl, alkylene, alkenylene and alkynylene group is optionally fluoro-substituted. In some embodiments, Ring C is substituted with one additional substituent selected from C₁₋₄alkyleneNR^{o}R^{p} and OC₁₋₄alkyleneR^{o}R^{p}, wherein R^{o} is selected from H and C₁₋₆alkyl and R^{p} is C₁₋₆alkyleneOC₁₋₆alkyl, and each alkyl and alkylene group is optionally fluoro-substituted. In some embodiments, Ring C is substituted with OC₁₋₄alkyleneR^{o}R^{p}, wherein R^{o} is selected from H and C₁₋₆alkyl and R^{p} is selected from C₁₋₆alkyleneOC₁₋₆alkyl, C₂₋₆alkenyleneOC₁₋₆alkyl, C₁₋₆alkynyleneOC₁₋₆alkyl, C₁₋₆alkyleneOC₁₋₆fluoroalkyl, C₂₋₆alkenyleneOC₁₋₆fluoroalkyl, and C₁₋₆alkynyleneOC₁₋₆fluoroalkyl. In some embodiments, Ring C is substituted with OC₁₋₄alkyleneR^{o}R^{p}, wherein R^{o} is selected from H and C₁₋₆alkyl and R^{p} is C₁₋₆alkyleneOC₁₋₆alkyl. In some embodiments, Ring C is substituted OC₁₋₄alkyleneR^{o}R^{p}, wherein R^{o} is selected from H and C₁₋₄alkyl and R^{p} is C₁₋₄alkyleneOC₁₋₄alkyl or C₁₋₄alkyleneOC₁₋₄fluoroalkyl. In some embodiments, R^{p} is selected from CH₂OCH₃, CH₂OCF₃, CH₂OCF₂H, CH₂CH₂OCH₃, CH₂CH₂OCF₂H and CH₂CH₂OCF₃. In some embodiments, R^{b} is selected from CH₂CH₂OCH₃, CH₂CH₂OCF₂H and CH₂CH₂OCF₃. In some embodiments, R^{p} is selected from CH₂CH₂OCH₃ and CH₂CH₂OCF₂H.

In some embodiments, Ring C is substituted with one additional substituent selected from C₁₋₄alkyleneNR^{o}R^{p} and OC₁₋₄alkyleneNR^{o}R^{p}, wherein R^{o} and R^{p} are joined to form, together with the atoms therebetween, a 3-6 membered saturated or unsaturated ring, optionally containing one additional heteroatom selected from NR^{t}, O S, SO and SO₂, and each alkylene group is optionally fluoro-substituted. In some embodiments, Ring C is substituted with one additional substituent selected from C₁₋₄alkyleneNR^{o}R^{p} and OC₁₋₄alkyleneNR^{o}R^{p}, wherein R^{o} and R^{p} are joined to form, together with the atoms therebetween, a 3 or 4 membered saturated or unsaturated ring, optionally containing one additional heteroatom selected from NR^{t}, O S, SO and SO₂, and each alkylene group is optionally fluoro-substituted. In some embodiments, R^{o} and R^{p} are joined to form, together with the atoms therebetween, aziranyl, azetidinyl or diazetidinyl. In some embodiments, Ring C is substituted with one additional substituent selected from C₂₋₄alkyleneNR^{o}R^{p} and OC₂₋₄alkyleneR^{o}R^{p}, wherein R^{o} and R^{p} are joined to form, together with the atoms therebetween, a 5 or 6 membered saturated or unsaturated ring, optionally containing one additional heteroatom selected from NR^{t}, O S, SO and SO₂, and each alkylene group is optionally fluoro-substituted. In some embodiments, R^{o} and R^{p} are joined to form, together with the atoms therebetween, pyrrolidinyl, imidazolidinyl, pyrazolidinyl, thiazolidinyl, isothiazolidinyl, piperidinyl, piperazinyl, thiomorpholinyl, or morpholinyl. In some embodiments, R^{o} and R^{p} are joined to form, together with the atoms therebetween, piperidinyl, piperazinyl, or morpholinyl. In some embodiments, R^{o} and R^{p} are joined to form, together with the atoms therebetween, piperazinyl or morpholinyl. In some embodiments, R^{o} and R^{p} are joined to form, together with the atoms therebetween, pyrrolidinyl.

In some embodiments, Ring C is substituted with one additional substituent selected from C₁₋₄alkyleneNR^{o}R^{p} and OC₁₋₄alkyleneNR^{o}R^{p}, and R^{o} and R^{p} are joined to form, together with the atoms therebetween, a 7-10 membered saturated or unsaturated ring, optionally containing one additional heteroatom selected from NR^{t}, O S, SO and SO₂, and each alkenylene group is optionally fluoro-substituted. In some embodiments, R^{o} and R^{p} are joined to form, together with the atoms therebetween, azepanyl, axepinyl, diazepinyl, oxazepanyl, thiazepinyl, azocanyl, azocinyl, azonanyl or azocinyl. In some embodiments, R^{o} and R^{p} are joined to form, together with the atoms therebetween, azepanyl, axepinyl, diazepinyl, or oxazepanyl. In some embodiments, R^{o} and R^{p} are joined to form, together with the atoms therebetween, oxazepanyl.

In some embodiments, R^{t} is selected from H, D, CHO, CO₂H, CONH₂, C₁₋₄alkyl, C₁₋₄alkenyl, C₁₋₄alkynyl, C(O)C₁₋₆alkyl, CO₂C₁₋₄alkyl, OC₁₋₄alkyl, CONHC₁₋₄alkyl, CON(C₁₋₄alkyl)(C₁₋₄alkyl), C₁₋₄alkyleneOC₁₋₄alkyl, C₂₋₄alkenyleneOC₁₋₄alkyl, and C₂₋₄alkynyleneOC₁₋₄alkyl, and each alkyl, alkenyl, alkynyl, alkylene, alkenylene and alkynylene group is optionally fluoro-substituted. In some embodiments. In some embodiments, R^{t} is selected from H, D, C₁₋₄alkyl, C₁₋₄alkenyl, C₁₋₄alkynyl, C(O)C₁₋₄alkyl, CO₂C₁₋₄alkyl, CONHC₁₋₄alkyl, C₁₋₄alkyleneOC₁₋₄alkyl, and each alkyl, alkenyl, alkynyl, alkylene, alkenylene and alkynylene group is optionally fluoro-substituted. In some embodiments, R^{t} is selected from H, D, C₁₋₄alkyl, and C(O)C₁₋₄alkyl, and each alkyl group is optionally fluoro-substituted. In some embodiments, R^{t} is selected from H, D, CH₃, CF₃, CHF₂, C(O)CH₃, C(O)CF₃, and C(O)CHF₂. In some embodiments, R^{s} is selected from H, D, CH₃, CF₃, and CHF₂. In some embodiments, R^{t} is selected from C(O)CH₃, C(O)CF₃, and C(O)CHF₂. In some embodiments, R^{t} is C(O)CH₃.

In some embodiments, R^{o} and R^{p} are joined to form, together with the atoms therebetween, a 5 or 6 membered saturated or unsaturated ring or a 7-10-membered saturated or unsaturated ring, and the 5 or 6 membered saturated or unsaturated ring or the 7-10-membered saturated or unsaturated ring optionally contain one additional heteroatom selected from NR^{t}, O, S, SO and SO₂, and are substituted with one or more of D, OH, NH₂, CHO, CO₂H, CONH₂, C₁₋₆alkyl, C₁₋₆alkenyl, C₁₋₆alkynyl, C(O)C₁₋₄alkyl, CO₂C₁₋₄alkyl, CONHC₁₋₄alkyl C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl, and each alkyl, alkenyl, alkynyl, alkylene, alkenylene and alkynylene group is optionally fluoro-substituted. In some embodiments, R^{o} and R^{p} are joined to form, together with the atoms therebetween, a 5 or 6 membered saturated or unsaturated ring or a 7-10-membered saturated or unsaturated ring, and the 5 or 6 membered saturated or unsaturated ring or the 7-10-membered saturated or unsaturated ring optionally contain one additional heteroatom selected from NR^{t}, O, S, SO and SO₂, and are substituted with one or more of D, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, C₁₋₆alkyleneOC₁₋₆alkyl, C₂₋₆alkenyleneOC₁₋₆alkyl, and C₂₋₆alkynyleneOC₁₋₆alkyl, and each alkyl, alkenyl, alkynyl, alkylene, alkenylene and alkynylene group is optionally fluoro-substituted.

In some embodiments, R^{o} and R^{p} are joined to form, together with the atoms therebetween, a 5 or 6 membered saturated or unsaturated ring or a 7-10-membered saturated or unsaturated ring, and the 5 or 6 membered saturated or unsaturated ring or the 7-10-membered saturated or unsaturated ring optionally contain one additional heteroatom selected from NR^{t}, O, S, SO and SO₂, and are substituted with one or more of D, C₁₋₆alkyl, C(O)C₁₋₆alkyl, OC₁₋₆alkyl, or C₁₋₆alkyleneOC₁₋₆alkyl, and each, alkyl, and alkylene group is optionally fluoro-substituted.

In some embodiments, R^{o} and R^{p} are joined to form, together with the atoms therebetween, a 5 or 6 membered saturated or unsaturated ring, optionally containing one additional heteroatom selected from NR^{t}, O, S, SO and SO₂, and substituted with one or more of D. In some embodiments, R^{o} and R^{p} are joined to form, together with the atoms therebetween, deutero-substituted piperidinyl, piperazinyl, or morpholinyl. In some embodiments, R^{o} and R^{p} are joined to form, together with the atoms therebetween, deutero-substituted morpholinyl. In some embodiments, R^{o} and R^{p} are joined to form, together with the atoms therebetween, a 5 or 6 membered saturated or unsaturated ring that is substituted with C(O)C₁₋₆alkyl that is optionally fluoro-substituted, suitably C(O)C₁₋₄alkyl that is optionally fluoro-substituted, more suitably C(O)CH₃, C(O)CHF₂, or C(O)CHF₃, most suitably C(O)CH₃. In some embodiments, R^{o} and R^{p} are joined to form, together with the atoms therebetween, a 5 or 6 membered saturated or unsaturated ring, optionally containing one additional heteroatom selected from NR^{t}, O, S, SO and SO₂, and substituted with C₁₋₆alkyleneOC₁₋₆alkyl that is optionally fluoro-substituted, suitably C₁₋₄alkyleneOC₁₋₄alkyl that is optionally fluoro-substituted, more suitably CH₂OCH₃, CH₂OCF₃, CH₂OCF₂H, CH₂CH₂OCH₃, CH₂CH₂OCF₂H and CH₂CH₂OCF₃, most suitably CH₂OCH₃, CH₂OCF₃, and CH₂OCF₂H.

In some embodiments, R^{o} and R^{p} are joined to form, together with the atoms therebetween, a 3-10 membered saturated or unsaturated ring, containing one additional heteroatom selected from NR^{t}, O, and S. In some embodiments, R^{o} and R^{p} are joined to form, together with the atoms therebetween, a 3-10 membered saturated or unsaturated ring, containing one additional heteroatom selected from S, SO and SO₂. In some embodiments, R^{o} and R^{p} are joined to form, together with the atoms therebetween, a 3-10 membered saturated or unsaturated ring, containing one additional heteroatom selected from NR^{t} and O.

In an embodiment, Ring C has no additional substituent.

In some embodiments, Ring C is pyridine and R⁷ has one or the following structures:

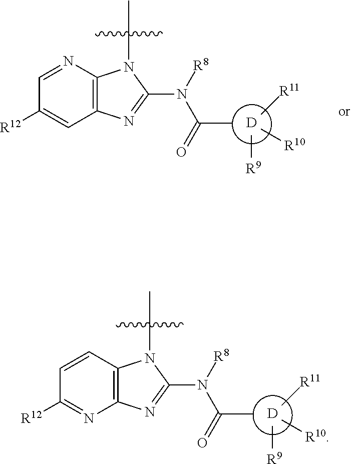

In some embodiments, R⁷ has the following structure:

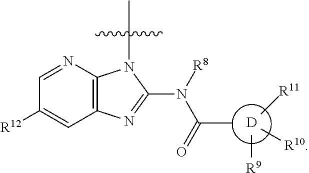

In some embodiments, Ring C is thienyl and R⁷ has one of the following structures:

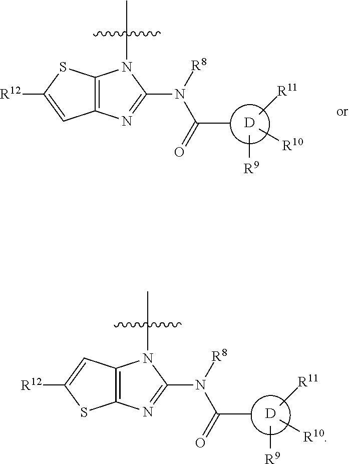

In some embodiments, R⁷ has the following structure:

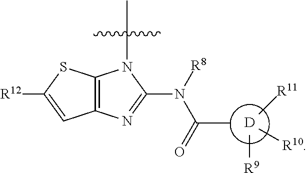

In some embodiments, R² is selected from H and C₁₋₄alkyl. In an embodiment, R² is H.

In some embodiments, R⁸ is selected from H and C₁₋₄alkyl. In an embodiment, R⁸ is H.

In some embodiments, Ring B is a 5-membered heteroaromatic ring comprising 1 to 3 heteroatoms selected from O, N and S. In some embodiments, Ring B is selected from thienyl, furanyl, pyrrolyl, thiazolyl, oxazolyl and pyrazolyl.

In some embodiments, Ring B is selected from thienyl and pyrazolyl. In an embodiment, Ring B is pyrazolyl.

In some embodiments, the compound of Formula I has the following structure:

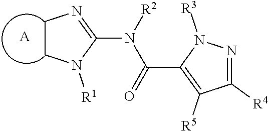

wherein R¹-R⁵ are as defined above.

In some embodiments, Ring D is a 5-membered heteroaromatic ring comprising 1 to 3 heteroatoms selected from O, N and S. In some embodiments, Ring D is selected from thienyl, furanyl, pyrrolyl, thiazolyl, oxazolyl and pyrazolyl. In some embodiments, Ring D is selected from thienyl and pyrazolyl. In an embodiment, Ring D is pyrazolyl.

In some embodiments, R⁷ has the following structure:

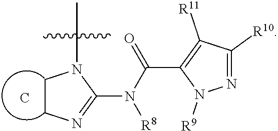

In some embodiments, R³, R⁴ and R⁵ are independently selected from H, C₁₋₆alkyl and C₁₋₆fluoroalkyl. In some embodiments, R³ and R⁴ are independently selected from H, C₁₋₆alkyl and C₁₋₆fluoroalkyl and R⁵ is H.

In some embodiments, R⁹, R¹⁰ and R¹¹ are independently selected from H, C₁₋₆alkyl and C₁₋₆fluoroalkyl. In some embodiments, R⁹ and R¹⁰ are independently selected from H, C₁₋₆alkyl and C₁₋₆fluoroalkyl, and R¹¹ is H.

In some embodiments, the compound of Formula I has the following structure:

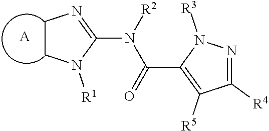

wherein
Ring A is a 5 or 6-membered heteroaromatic ring comprising 1 to 3 heteroatoms selected from O, N and S that is substituted with at least one R⁶ and optionally substituted with one or two additional substituents independently selected from halo, C₁₋₆alkyl, C₂₋₆alkenyl, C₂₋₆alkynyl, OR^{a}, NR^{a}R^{b}, C₁₋₆alkyleneOR^{a}, C₁₋₆alkyleneNR^{a}R^{b}, OC₁₋₆alkyleneOR^{a}, OC₁₋₆alkyleneNR^{a}R^{b}, C₂₋₆alkenyleneOR^{a}, C₂₋₆alkenyleneNR^{a}R^{b}, OC₂₋₆alkenyleneOR^{a}, OC₂₋₆alkenyleneNR^{a}R^{b} C₂₋₆alkynyleneOR^{a}, C₂₋₆alkynyleneNR^{a}R^{b}, OC₂₋₆alkynyleneOR^{a} and OC₂₋₆alkynyleneNR^{a}R^{b};
R^{a} and R^{b} are independently selected from H and C₁₋₆alkyl, or R^{a} and R^{b} are joined to form, together with the atoms therebetween, a 5 or 6 membered saturated or unsaturated ring, optionally containing one additional heteroatom selected from NR^{s}, O, S, SO and SO₂;
R^{s} is selected from H and C₁₋₆alkyl;
R¹ is selected from C₁₋₁₀alkyleneR⁷, C₂₋₁₀alkenyleneR⁷, C₂₋₁₀alkynyleneR⁷, C₃₋₁₀cycloalkyl, 4-10-membered heterocycloalkyl, 5-10-membered heteroaryl and C₆₋₁₀aryl, the latter 4 groups and each C₁₋₁₀alkylene, C₂₋₁₀alkenylene and C₂₋₁₀alkynylene being optionally substituted with 1-4 substituents independently selected from halo, nitro, C₁₋₆alkyleneR^{c}, C₁₋₆alkyleneOR^{c}, OC₁₋₆alkyleneOR^{c}, OR^{c}, NR^{c}R^{d}, CO₂R^{c}, CONR^{c}R^{d}, SO₂NR^{c}R^{d}, OCONR^{c}R^{d}, COR^{e}, C₃₋₁₀cycloalkyl, 3-10-membered heterocycloalkyl, 5-10-membered heteroaryl and C₆₋₁₀aryl;
R^{c} is selected from H, C₁₋₆alkyl, COC₁₋₆alkyl, C₃₋₁₀cycloalkyl, 3-10-membered heterocycloalkyl, 5-10-membered heteroaryl and C₆₋₁₀aryl;
R^{d} is selected from H and C₁₋₆alkyl;
R^{e} is selected from H, C₁₋₆alkyl, C₃₋₁₀cycloalkyl, 3-10-membered heterocycloalkyl, 5-10-membered heteroaryl and C₆₋₁₀aryl;
R² is selected from H, C₁₋₆alkyl, C₂₋₆alkenyl and C₂₋₆alkynyl;
R³ is selected from H, C₁₋₆alkyl, C₃₋₁₀cycloalkyl and 3-10-membered heterocycloalkyl, the latter three groups being optionally substituted with halo, OR^{f}, NR^{f}R^{g}, COR^{f}, CO₂R^{f}, CONR^{f}R^{g}, SO₂NR^{f}R^{g}, NHSO₂R^{f} and OCONR^{f}R^{g};
R^{f} and R^{g} are independently selected from H and C₁₋₆alkyl;
R⁴ is selected from H, C₁₋₆alkyl, C₂₋₆alkenyl, C₂₋₆alkynyl and C₃₋₇cycloalkyl;
R⁵ is selected from H, halo and C₁₋₆alkyl;
R⁶ is selected from COR^{h}, CO₂R^{h}, CONR^{h}Rⁱ, OCONR^{h}Rⁱ, NHSO₂R^{h}, SO₂NR^{h}Rⁱ, NHCOR^{h} and NHCO₂R^{h},
R^{h} and Rⁱ are independently selected from H and C₁₋₆alkyl;
R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₁₀cycloalkyl, 4-10-membered heterocycloalkyl, 5-10-membered heteroaryl and C₆₋₁₀aryl, the latter 4 groups being optionally substituted with 1-4 substituents independently selected from halo, nitro, C₁₋₆alkyleneR^{m}, C₁₋₆alkyleneOR^{m}, OC₁₋₆alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl, or R⁷ is

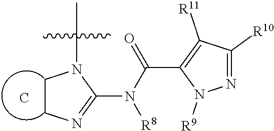

R^{j} is selected from H, C₁₋₆alkyl, COC₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{k} is selected from H and C₁₋₆alkyl;
R^{l} is selected from H, C₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{m} is selected from H and C₁₋₆alkyl;
Rⁿ is selected from H, C₁₋₆alkyl, C₃₋₁₀cycloalkyl, 4-10-membered heterocycloalkyl, 5-10-membered heteroaryl and C₆₋₁₀aryl;
R⁸, R⁹, R¹⁰ and R¹¹ are, independently, as defined for R², R³, R⁴ and R⁵, respectively;
Ring C is selected from phenyl and a 5 or 6-membered heteroaromatic ring comprising 1 to 3 heteroatoms selected from O, N and S, and Ring C is substituted with at least one R¹² and optionally substituted with one or two additional substituents independently selected from halo, C₁₋₆alkyl, C₂₋₆alkenyl, C₂₋₆alkynyl, OR^{o}, NR^{o}R^{p}, C₁₋₆alkyleneOR^{o}, C₁₋₆alkyleneNR^{o}R^{p}, OC₁₋₆alkyleneNR^{o}R^{p}, OC₁₋₆alkyleneOR^{o}, C₂₋₆alkenyleneOR^{o}, C₂₋₆alkenyleneNR^{o}R^{p}, OC₂₋₆alkenyleneNR^{o}R^{p}, OC₂₋₆alkenyleneOR^{o}, C₂₋₆alkynyleneOR^{o}, C₂₋₆alkynyleneNR^{o}R^{p}, OC₂₋₆alkynyleneOR^{o} and OC₂₋₆alkynyleneNR^{o}R^{p};
R^{o} and R^{p} are independently selected from H and C₁₋₆alkyl, or R^{o} and R^{p} are joined to form, together with the atoms therebetween, a 5 or 6 membered saturated or unsaturated ring, optionally containing one additional heteroatom selected from NR^{t}, O, S, SO and SO₂,
R^{t} is selected from H and C₁₋₆alkyl;
R¹² is selected from COR^{a}, CO₂R^{q}, CONR^{q}R^{r}, OCONR^{q}R^{r}, NHSO₂R^{q}, SO_{2N}R^{q}R^{r}, NHCOR^{q} and NHCO₂R^{q};
R^{q} and R^{r} are independently selected from H and C₁₋₆alkyl; and
each alkyl, alkenyl, alkynyl, alkylene, alkenylene and alkynylene group is optionally fluoro-substituted.

In some embodiments, the compound of Formula I is selected from the compounds listed below:

| Compound ID | Structure |
|---|---|
| A | 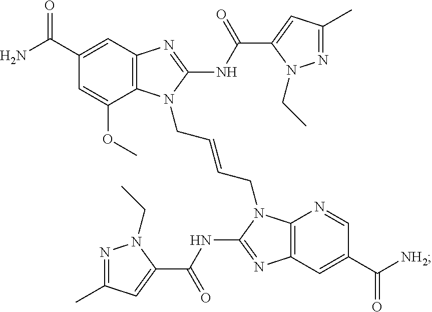 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OC)c21] |
| B | 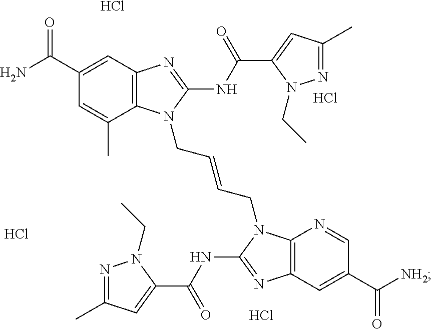 [SMILES: Cl ; Cl ; CCn1nc(C)cc1C(=O)Nc1nc2cc(C(N)=O)cnc2n1C/C=C/Cn1c(NC(=O)c2cc(C)nn2CC)nc2cc(C(N)=O)cc(C)c21 ; Cl ; Cl] |
| C | 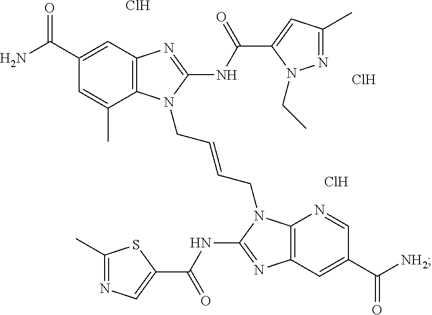 [SMILES: Cl ; CCn1nc(C)cc1C(=O)Nc1nc2cc(C(N)=O)cc(C)c2n1C/C=C/Cn1c(NC(=O)c2cnc(C)s2)nc2cc(C(N)=O)cnc21 ; Cl ; Cl] |
| D | 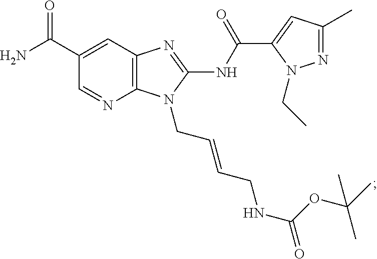 [SMILES: CCn1nc(C)cc1C(=O)Nc1nc2cc(C(N)=O)cnc2n1C/C=C/CNC(=O)OC(C)(C)C] |
| E | 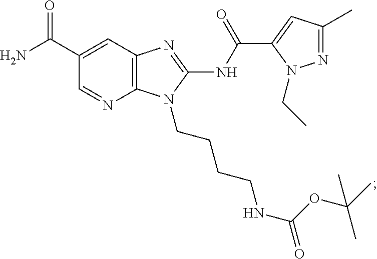 [SMILES: CCn1nc(C)cc1C(=O)Nc1nc2cc(C(N)=O)cnc2n1CCCCNC(=O)OC(C)(C)C] |
| F | 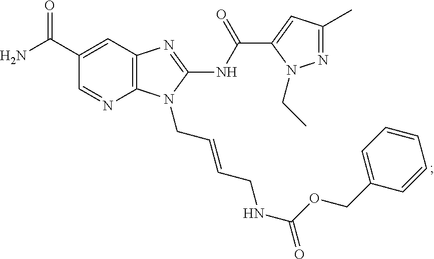 [SMILES: CCn1nc(C)cc1C(=O)Nc1nc2cc(C(N)=O)cnc2n1C/C=C/CNC(=O)OCc1ccccc1] |
| G | 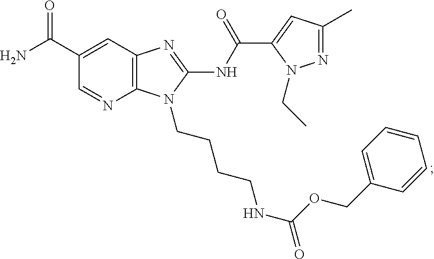 [SMILES: CCn1nc(C)cc1C(=O)Nc1nc2cc(C(N)=O)cnc2n1CCCCNC(=O)OCc1ccccc1] |
| H | 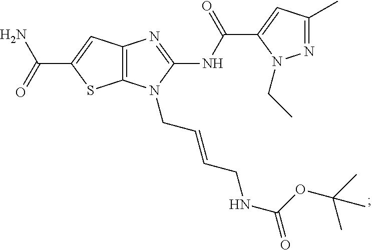 [SMILES: CCn1nc(C)cc1C(=O)Nc1nc2cc(C(N)=O)sc2n1C/C=C/CNC(=O)OC(C)(C)C] |
| J | 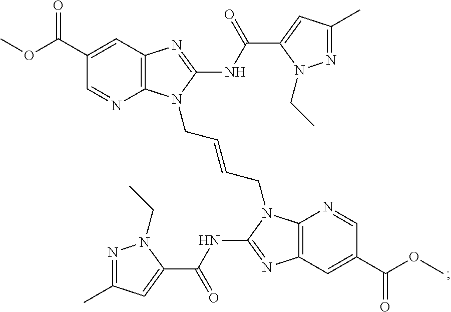 |
| K | 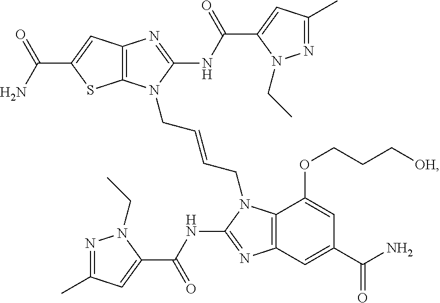 [SMILES: CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCO)c21] |
| L | 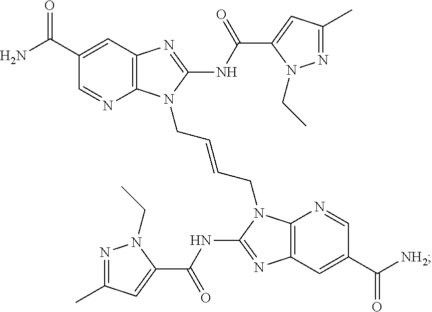 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cnc21] |
| M | 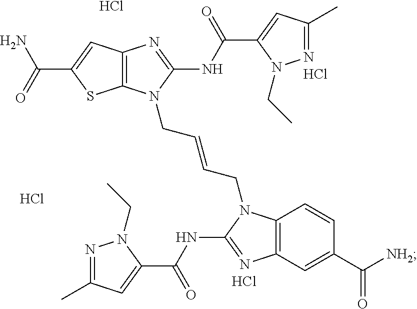 [SMILES: Cl ; CCn1nc(C)cc1C(=O)Nc1nc2cc(C(N)=O)ccc2n1C/C=C/Cn1c(NC(=O)c2cc(C)nn2CC)nc2cc(C(N)=O)sc21 ; Cl ; Cl ; Cl] |
| N | 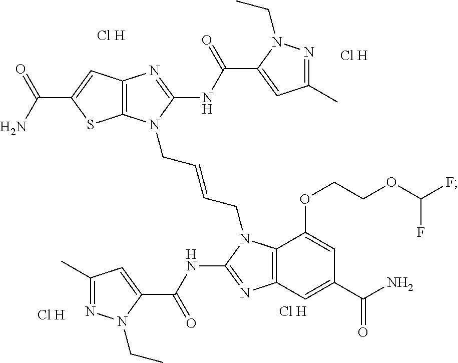 |
| O | 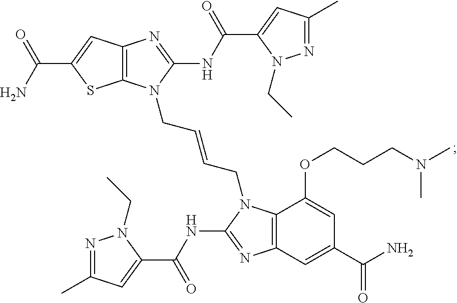 [SMILES: CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCN(C)C)c21] |
| P | 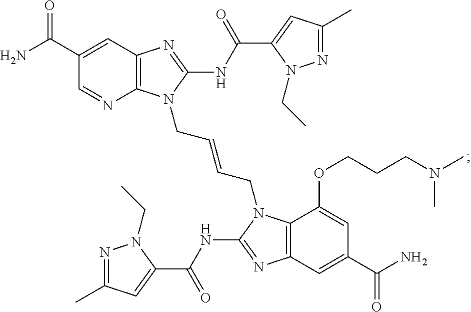 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN(C)C)c21] |
| Q | 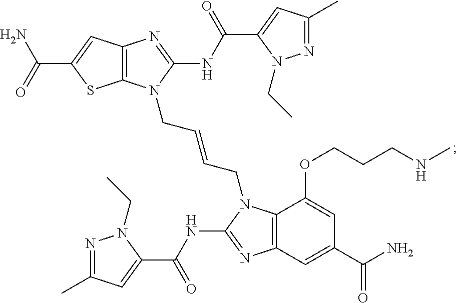 [SMILES: CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCNC)c21] |
| R | 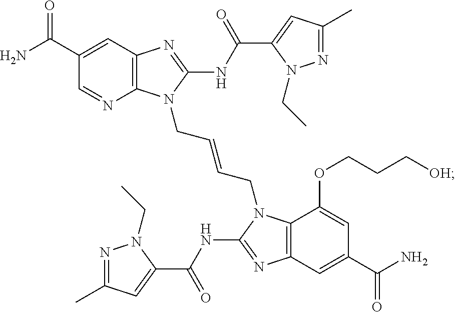 |
| S | 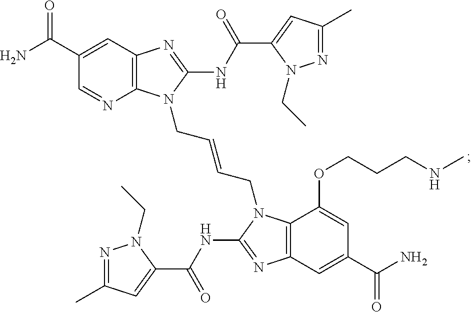 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNC)c21] |
| T | 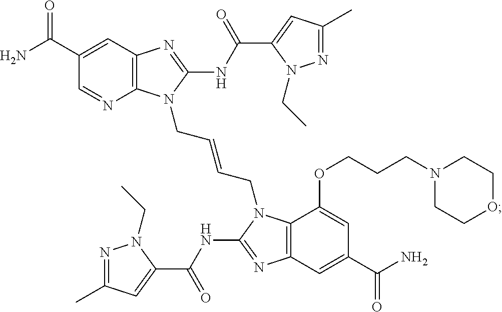 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21] |
| U | 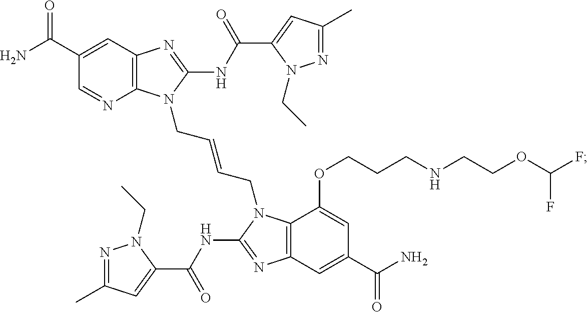 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNCCOC(F)F)c21] |
| V | 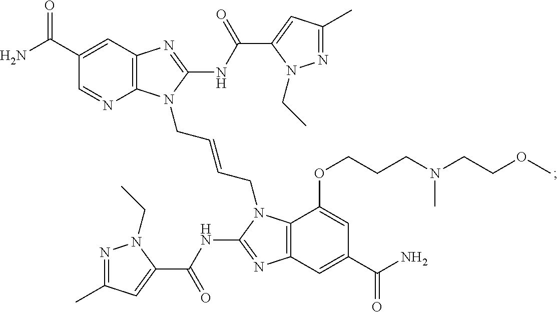 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN(C)CCOC)c21] |
| W | 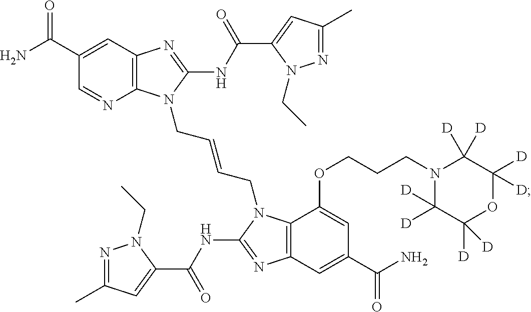 |
| X | 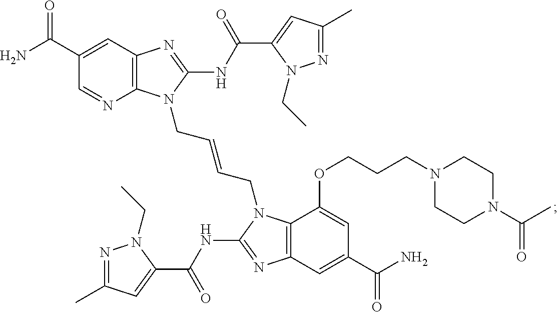 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCN(C(C)=O)CC3)c21] |
| Y | 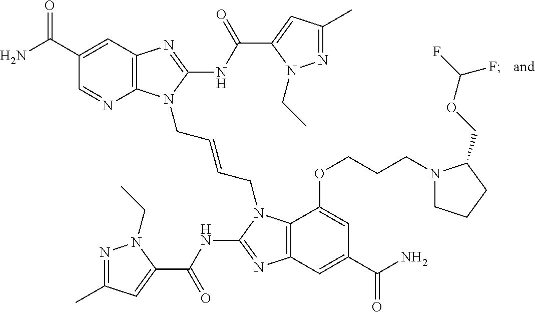 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC[C@H]3COC(F)F)c21] |
| Z | 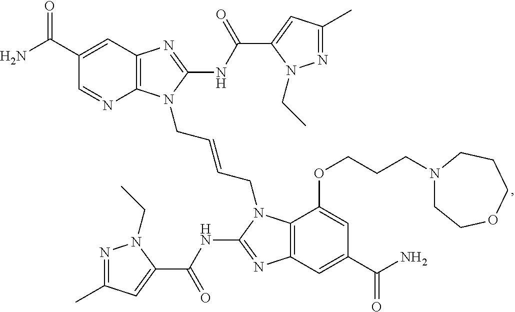 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCCOCC3)c21] |

or a pharmaceutically acceptable salt, solvate and/or prodrug thereof.

The present application also includes compounds that are a specific subset of compounds of Formula I, that is compounds of Formula II or a pharmaceutically acceptable salt, solvate and/or prodrug thereof:

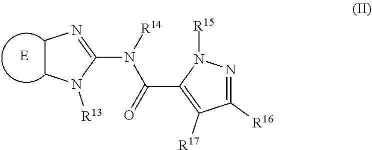

wherein:
Ring E is a 5 or 6-membered heteroaromatic ring comprising 1 to 3 heteroatoms selected from O, N and S that is substituted with at least one R¹⁸ and optionally substituted with one or two additional substituents independently selected from OC₁₋₆alkyleneNR¹⁹R²⁰, OC₂₋₆alkenyleneNR¹⁹R²⁰, OC₂₋₆alkynyleneNR¹⁹R²⁰, OC₁₋₆alkyleneNR²¹C₁₋₆alkyleneOC₁₋₆alkyl, OC₂₋₆alkenyleneNR²¹C₁₋₆alkyleneOC₁₋₆alkyl, OC₂₋₆alkynyleneNR²¹C₁₋₆alkyleneOC₁₋₆alkyl, OC₁₋₆alkyleneNR²¹C₂₋₆alkenyleneOC₁₋₆alkyl, OC₂₋₆alkenyleneNR²¹C₂₋₆alkenyleneOC₁₋₆alkyl, OC₂₋₆alkynyleneNR²¹C₂₋₆alkenyleneOC₁₋₆alkyl, OC₁₋₆alkyleneNR²¹C₂₋₆alkynyleneOC₁₋₆alkyl, OC₂₋₆alkenyleneNR²¹C₂₋₆alkynyleneOC₁₋₆alkyl, and OC₂₋₆alkynyleneNR²¹C₂₋₆alkynyleneOC₁₋₆alkyl;
R¹³ is selected from C₁₋₁₀alkyleneR²², C₂₋₁₀alkenyleneR²² and C₂₋₁₀alkynyleneR²²;
R¹⁴ is selected from H, C₁₋₆alkyl, C₂₋₆alkenyl and C₂₋₆alkynyl;
R¹⁵ is selected from H, C₁₋₆alkyl, C₃₋₁₀cycloalkyl and 3-10-membered heterocycloalkyl, the latter three groups being optionally substituted with halo, OR²³, NR²³R²⁴, COR²³, CO₂R²³, CONR²³R²⁴, SO₂NR²³R²⁴, NHSO₂R²³ and OCONR²³R²⁴;
R¹⁶ is selected from H, C₁₋₆alkyl, C₂₋₆alkenyl, C₂₋₆alkynyl and C₃₋₇cycloalkyl;
R¹⁷ is selected from H, halo and C₁₋₆alkyl;
R¹⁸ is selected from COR²⁵, CO₂R²⁵, CONR²⁵R²⁶, OCONR²⁵R²⁶, NHSO₂R²⁵, SO₂NR²⁵R²⁶, NHCOR²⁵ and NHCO₂R²⁵,
R¹⁹ and R²⁰ are joined to form, together with the nitrogen therebetween, a 3-6 membered saturated or unsaturated ring, optionally containing one additional heteroatom selected from NR²⁷, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl) or C₁₋₆alkyleneOC₁₋₆alkyl; or
R¹⁹ and R²⁰ are joined to form, together with the nitrogen therebetween, a 7-10 membered saturated or unsaturated ring, optionally containing one additional heteroatom selected from NR²⁷, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R²³, R²⁴, R²⁵ and R²⁶ are independently selected from H and C₁₋₆alkyl;
R²⁷ is selected from D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;

### R²² is

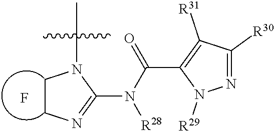

R²⁸, R²⁹, R³⁰ and R³¹ are, independently, as defined for R¹⁴, R¹⁵, R¹⁶ and R¹⁷, respectively;
Ring F is selected from phenyl and a 5 or 6-membered heteroaromatic ring comprising 1 to 3 heteroatoms selected from O, N and S, and Ring F is substituted with at least one R³² and substituted with one or two additional substituents independently selected from OC₁₋₆alkyleneNR³³R³⁴, OC₂₋₆alkenyleneNR³³R³⁴, OC₂₋₆alkynyleneNR³³R³⁴, OC₁₋₆alkyleneNR³⁵C₂₋₆alkenyleneOC₁₋₆alkyl, OC₁₋₆alkyleneNR³⁵C₁₋₆alkyleneOC₁₋₆alkyl, OC₂₋₆alkenyleneNR³⁵C₁₋₆alkyleneOC₁₋₆alkyl, OC₂₋₆alkynyleneNR³⁵C₁₋₆alkyleneOC₁₋₆alkyl, OC₁₋₆alkyleneNR³⁵C₂₋₆alkenyleneOC₁₋₆alkyl, OC₂₋₆alkenyleneNR³⁵C₂₋₆alkenyleneOC₁₋₆alkyl, OC₂₋₆alkynyleneNR³⁵C₂₋₆alkenyleneOC₁₋₆alkyl, OC₁₋₆alkyleneNR³⁵C₂₋₆alkynyleneOC₁₋₆alkyl, OC₂₋₆alkenyleneNR³⁵C₂₋₆alkynyleneOC₁₋₆alkyl, and OC₂₋₆alkynyleneNR³⁵C₂₋₆alkynyleneOC₁₋₆alkyl;
R³² is selected from COR³⁶, CO₂R³⁶, CONR³⁶R³⁷, OCONR³⁶R³⁷, NHSO₂R³⁶, SO₂NR³⁶R³⁷, NHCOR³⁶ and NHCO₂R³⁶;
R³³ and R³⁴ are joined to form, together with the nitrogen therebetween, a 3-6 membered saturated or unsaturated ring, optionally containing one additional heteroatom selected from NR³⁸, O, S, SO and SO₂, and substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl) or C₁₋₆alkyleneOC₁₋₆alkyl; or
R³³ and R³⁴ are joined to form, together with the nitrogen therebetween, a 7-10 membered saturated or unsaturated ring, optionally containing one additional heteroatom selected from NR³⁸, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R³⁵, R³⁶, and R³⁷ are independently selected from H and C₁₋₆alkyl;
R³⁸ is selected from D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
and
each alkyl, alkenyl, alkynyl, alkylene, alkenylene and alkynylene group is optionally fluoro-substituted.

In some embodiments, Ring E is selected from pyridinyl, pyrimidinyl, pyridazinyl, pyrazinyl, triazinyl, tetrazinyl, thienyl, furanyl, pyrrolyl, thiazolyl, oxazolyl and pyrazolyl. In some embodiments, Ring E is selected from pyridinyl, thienyl and furanyl. In some embodiments, Ring E is pyridinyl or thienyl. In some embodiments, Ring E is pyridinyl.

In some embodiments, Ring E is substituted with one R¹⁸ and optionally substituted with one additional substituent selected from OC₁₋₆alkyleneNR¹⁹R²⁰, OC₂₋₆alkenyleneNR¹⁹R²⁰, OC₂₋₆alkynyleneNR¹⁹R²⁰, OC₁₋₆alkyleneNR²¹C₂₋₆alkenyleneOC₁₋₆alkyl, OC₁₋₆alkyleneNR²¹C₁₋₆alkyleneOC₁₋₆alkyl, OC₂₋₆alkenyleneNR²¹C₁₋₆alkyleneOC₁₋₆alkyl, OC₂₋₆alkynyleneNR²¹C₁₋₆alkyleneOC₁₋₆alkyl, OC₁₋₆alkyleneNR²¹C₂₋₆alkenyleneOC₁₋₆alkyl, OC₂₋₆alkenyleneNR²¹C₂₋₆alkenyleneOC₁₋₆alkyl, OC₂₋₆alkynyleneNR²¹ 02-6alkenyleneOC₁₋₆alkyl, OC₁₋₆alkyleneNR²¹C₂₋₆alkynyleneOC₁₋₆alkyl, OC₂₋₆alkenyleneNR²¹C₂₋₆alkynyleneOC₁₋₆alkyl, and OC₂₋₆alkynyleneNR²¹C₂₋₆alkynyleneOC₁₋₆alkyl, and each alkyl, alkenyl, alkynyl, alkylene, alkenylene and alkynylene group is optionally fluoro-substituted. In some embodiments, Ring E is substituted with one R¹⁸ and optionally substituted with one additional substituent selected from OC₁₋₄alkyleneNR¹⁹R²⁰, OC₂₋₄alkenyleneNR¹⁹R²⁰, OC₂₋₄alkynyleneNR¹⁹R²⁰, OC₁₋₄alkyleneNR²¹C₁₋₄alkyleneOC₁₋₄alkyl, OC₂₋₄alkenyleneNR²¹C₁₋₄alkyleneOC₁₋₄alkyl, OC₂₋₄alkynyleneNR²¹C₁₋₄alkyleneOC₁₋₄alkyl, OC₁₋₄alkyleneNR²¹C₂₋₄alkenyleneOC₁₋₄alkyl, OC₂₋₄alkenyleneNR²¹C₂₋₄alkenyleneOC₁₋₆alkyl, OC₂₋₄alkynyleneNR²¹C₂₋₄alkenyleneOC₁₋₄alkyl, OC₁₋₄alkyleneNR²¹C₂₋₄alkynyleneOC₁₋₄alkyl, OC₂₋₄alkenyleneNR²¹C₂₋₄alkynyleneOC₁₋₄alkyl, and OC₂₋₄alkynyleneNR²¹C₂₋₄alkynyleneOC₁₋₄alkyl, and each alkyl, alkenyl, alkynyl, alkylene, alkenylene and alkynylene group is optionally fluoro-substituted.

In some embodiments, Ring E is substituted with one R¹⁸ and optionally substituted with one additional substituent selected from OC₁₋₄alkyleneNR¹⁹R²⁰ and OC₁₋₄alkyleneNR²¹C₁₋₄alkyleneOC₁₋₄alkyl and each alkyl and alkylene group is optionally fluoro-substituted.

In some embodiments, Ring E is substituted with one R¹⁸ and optionally substituted with OC₁₋₄alkyleneNR¹⁹R²⁰ and each alkyl and alkylene, group is optionally fluoro-substituted. In some embodiments, Ring E is substituted with one R¹⁸ and optionally substituted with one additional substituent selected from OCH₂NR¹⁹R²⁰, OCH₂CH₂NR¹⁹R²⁰ and OCH₂CH₂CH₂NR¹⁹R²⁰. In some embodiments, Ring E is substituted with one R¹⁸ and optionally substituted with OCH₂CH₂CH₂NR¹⁹R²⁰.

In some embodiments, Ring E is substituted with one additional substituent selected from C₁₋₄alkyleneNR¹⁹R²⁰ and OC₁₋₄alkyleneNR¹⁹R²⁰ wherein R¹⁹ and R²⁰ are joined to form, together with the atoms therebetween, a 3-6 membered saturated or unsaturated ring, optionally containing one additional heteroatom selected from NR²⁷, O S, SO and SO₂, and each alkylene group is optionally fluoro-substituted. In some embodiments, Ring E is substituted with one additional substituent selected from C₁₋₄alkyleneNR¹⁹R²⁰ and OC₁₋₄alkyleneNR¹⁹R²⁰, wherein R¹⁹ and R²⁰ are joined to form, together with the atoms therebetween, a 3 or 4 membered saturated or unsaturated ring, optionally containing one additional heteroatom selected from NR²⁷, O S, SO and SO₂, and each alkyl and alkylene group is optionally fluoro-substituted. In some embodiments, R¹⁹ and R²⁰ are joined to form, together with the atoms therebetween, aziranyl, azetidinyl or diazetidinyl. In some embodiments, R¹⁹ and R²⁰ are joined to form, together with the nitrogen therebetween, a 5 or 6 membered saturated or unsaturated ring selected from pyrrolidinyl, imidazolidinyl, pyrazolidinyl, thiazolidinyl, isothiazolidinyl, piperidinyl, piperazinyl, thiomorpholinyl, and morpholinyl. In some embodiments, R¹⁹ and R²⁰ are joined to form, together with the nitrogen therebetween, piperidinyl, piperazinyl, or morpholinyl. In some embodiments, R¹⁹ and R²⁰ are joined to form, together with the nitrogen therebetween, piperazinyl or morpholinyl. In some embodiments, R¹⁹ and R²⁰ are joined to form, together with the nitrogen therebetween, pyrrolidinyl.

In some embodiments, R¹⁹ and R²⁰ are joined to form, together with the nitrogen therebetween, a 7-10 membered saturated or unsaturated ring, optionally containing one additional heteroatom selected from NR²⁷, O, S, SO and SO₂. In some embodiments, R¹⁹ and R²⁰ are joined to form, together with the nitrogen therebetween, a 7-10 membered saturated or unsaturated ring selected from azepanyl, axepinyl, diazepinyl, oxazepanyl, thiazepinyl, azocanyl, azocinyl, azonanyl and azocinyl. In some embodiments, R¹⁹ and R²⁰ are joined to form, together with the nitrogen therebetween, azepanyl, axepinyl, diazepinyl, or oxazepanyl. In some embodiments, R¹⁹ and R²⁰ are joined to form, together with the nitrogen therebetween, oxazepanyl.

In some embodiments, R²⁷ is selected from H, D, CHO, CO₂H, CONH₂, C₁₋₄alkyl, C₁₋₆alkenyl, C₁₋₆alkynyl, C(O)C₁₋₆alkyl, CO₂C₁₋₄alkyl, OC₁₋₄alkyl, CONHC₁₋₄alkyl, CON(C₁₋₄alkyl)(C₁₋₄alkyl), C₁₋₄alkyleneOC₁₋₄alkyl, C₂₋₄alkenyleneOC₁₋₄alkyl, and C₂₋₄alkynyleneOC₁₋₄alkyl, and each alkyl, alkenyl, alkynyl, alkylene, alkenylene and alkynylene group is optionally fluoro-substituted. In some embodiments. In some embodiments, R²⁷ is selected from H, D, C₁₋₄alkyl, C₁₋₄alkenyl, C₁₋₄alkynyl, C(O)C₁₋₄alkyl, CO₂C₁₋₄alkyl, CONHC₁₋₄alkyl, C₁₋₄alkyleneOC₁₋₄alkyl, and each alkyl, alkenyl, alkynyl, alkylene, alkenylene and alkynylene group is optionally fluoro-substituted. In some embodiments, R²⁷ is selected from H, D, C₁₋₄alkyl, and C(O)C₁₋₄alkyl and each alkyl group is optionally fluoro-substituted. In some embodiments, R²⁷ is selected from H, D, CH₃, CF₃, CHF₂, C(O)CH₃, C(O)CF₃, and C(O)CHF₂. In some embodiments, R²⁷ is selected from H, D, CH₃, CF₃, and CHF₂. In some embodiments, R²⁷ is selected from C(O)CH₃, C(O)CF₃, and C(O)CHF₂. In some embodiments, R²⁷ is C(O)CH₃.

In some embodiments, R¹⁹ and R²⁰ are joined to form, together with the nitrogen therebetween, a 3-6 membered saturated or unsaturated ring or a 7-10 membered saturated or unsaturated ring, and the 3-6 membered saturated or unsaturated ring or the 7-10 membered saturated or unsaturated ring optionally containing one additional heteroatom selected from NR²⁷, O, S, SO and SO₂ and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, or C₂₋₆alkynyleneOC₁₋₆alkyl, suitably D, CHO, CO₂H, CONH₂, C₁₋₆alkyl, C(O)C₁₋₆alkyl, CO₂C₁₋₆alkyl, OC₁₋₆alkyl, C₁₋₆alkyleneOC₁₋₆alkyl, C₂₋₆alkenyleneOC₁₋₆alkyl, or C₂₋₆alkynyleneOC₁₋₆alkyl, more suitably one or more of D, CHO, C₁₋₆alkyl, C(O)C₁₋₆alkyl, OC₁₋₆alkyl, or C₁₋₆alkyleneOC₁₋₆alkyl, and each alkyl, alkenyl, alkynyl, alkylene, alkenylene and alkynylene group is optionally fluoro-substituted.

In some embodiments, R¹⁹ and R²⁰ are joined to form, together with the nitrogen therebetween, a 5 or 6 membered saturated or unsaturated ring, optionally containing one additional heteroatom selected from NR²⁷, O, S, SO and SO₂, and optionally substituted with one or more of D. In some embodiments, R¹⁹ and R²⁰ are joined to form, together with the nitrogen therebetween, optionally deutero-substituted piperidinyl, piperazinyl, or morpholinyl. In some embodiments, R¹⁹ and R²⁰ are joined to form, together with the nitrogen therebetween, deutero-substituted morpholinyl. In some embodiments, R¹⁹ and R²⁰ are joined to form, together with the nitrogen therebetween, a 5 or 6 membered saturated or unsaturated ring, optionally containing one additional heteroatom selected from NR²⁷, O, S, SO and SO₂, and optionally substituted with C(O)C₁₋₆alkyl or C(O)C₁₋₆fluoroalkyl, suitably C(O)C₁₋₄alkyl, or C(O)C₁₋₄fluoroalkyl more suitably C(O)CH₃, C(O)CHF₂, or C(O)CHF₃, even more suitably C(O)CH₃. In some embodiments, R¹⁹ and R²⁰ are joined to form, together with the nitrogen therebetween, a 5 or 6 saturated or unsaturated ring, optionally containing one additional heteroatom selected from NR²⁷, O, S, SO and SO₂, and optionally substituted with C₁₋₆alkyleneOC₁₋₆alkyl that is optionally fluoro-substituted, suitably C₁₋₄alkyleneOC₁₋₄alkyl, more suitably CH₂OCH₃, CH₂OCF₃, CH₂OCF₂H, CH₂CH₂OCH₃, CH₂CH₂OCF₂H or CH₂CH₂OCF₃, even more suitably CH₂OCH₃, CH₂OCF₃ or CH₂OCF₂H, and most suitably CH₂OCH₃ or CH₂OCF₂H.

In some embodiments, Ring E is substituted with one R¹⁸ and optionally substituted with OC₁₋₄alkyleneNR²¹C₁₋₄alkyleneOC₁₋₄alkyl wherein R²¹ is selected from H and C₁₋₄alkyl and each alkyl an alkylene group is optionally fluoro-substituted. In some embodiments, Ring E is substituted with one R¹⁸ and optionally substituted with one additional substituent selected from OCH₂CH₂CH₂NHCH₂CH₂OCH₃, OCH₂CH₂CH₂N(CH₃)CH₂CH₂OCH₃, OCH₂CH₂CH₂NHCH₂CH₂OCF₃, OCH₂CH₂CH₂N(CH₃)CH₂CH₂OCF₃, OCH₂CH₂CH₂NHCH₂CH₂OCHF₂ and OCH₂CH₂CH₂N(CH₃)CH₂CH₂OCHF₂. In some embodiments, Ring E is substituted with one R¹⁸ and optionally substituted with one additional substituent selected from OCH₂CH₂CH₂NHCH₂CH₂OCH₃, OCH₂CH₂CH₂N(CH₃)CH₂CH₂OCH₃, OCH₂CH₂CH₂NHCH₂CH₂OCHF₂, and OCH₂CH₂CH₂N(CH₃)CH₂CH₂OCHF₂.

In some embodiments, R¹⁸ is selected from COR²⁵, CO₂R²⁵ and CONR²⁵R²⁶; and R²⁵ and R²⁶ are independently selected from H, C₁₋₄alkyl and C₁₋₄fluoroalkyl. In some embodiments, R¹⁸ is selected from COR²⁵, CO₂R²⁵ and CONR²⁵R²⁶; and R²⁵ and R²⁶ are independently selected from H and CH₃. In some embodiments, R¹⁸ is CONR²⁵R²⁶; and R²⁵ and R²⁶ are independently selected from H and CH₃. In an embodiment, R¹⁸ is CONH₂.

In some embodiments, Ring E is pyridine and the compound of Formula II has one of the following structures:

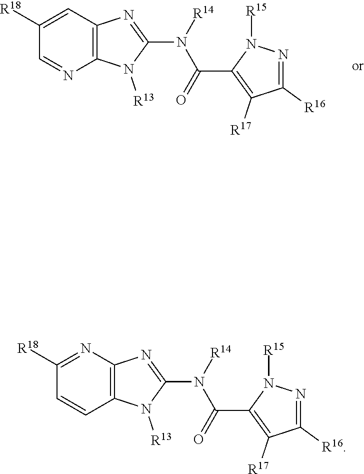

In some embodiments, the compound of Formula II has the following structure:

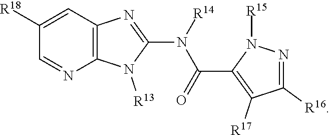

In some embodiments, Ring E is thienyl and the compound of Formula II has one of the following structures:

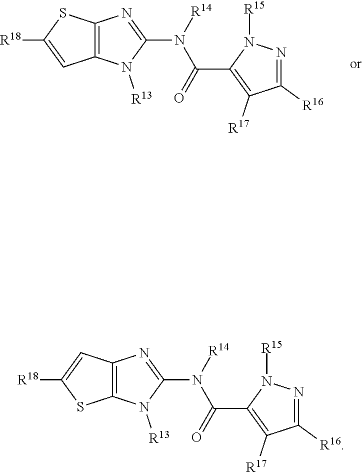

In some embodiments, the compound of Formula II has the following structure:

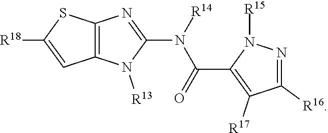

In some embodiments, R¹⁹ and R²⁰ are joined to form, together with the atoms therebetween, a 3-10 membered saturated or unsaturated ring, containing one additional heteroatom selected from NR²⁷, O, and S. In some embodiments, R¹⁹ and R²⁰ are joined to form, together with the atoms therebetween, a 3-10 membered saturated or unsaturated ring, containing one additional heteroatom selected from S, SO and SO₂. In some embodiments, R¹⁹ and R²⁰ are joined to form, together with the atoms therebetween, a 3-10 membered saturated or unsaturated ring, containing one additional heteroatom selected from NR²⁷ and 0.

In some embodiments, Ring E has no additional substituents.

In some embodiments, R¹³ is selected from C₁₋₆alkyleneR²², C₂₋₆alkenyleneR²² and C₂₋₆alkynyleneR²² and each alkylene, alkenylene and alkynylene group is optionally fluoro-substituted. In some embodiments, R¹³ is C₂₋₆alkenyleneR²². In some embodiments, R¹³ is C₄alkenyleneR²².

In some embodiments, Ring F is selected from phenyl, pyridinyl, pyrimidinyl, pyridazinyl, pyrazinyl, triazinyl, tetrazinyl, thienyl, furanyl, pyrrolyl, thiazolyl, oxazolyl, and pyrazolyl. In some embodiments, Ring F is selected from phenyl, pyridinyl, thienyl and furanyl. In some embodiments, Ring F is phenyl, pyridinyl or thienyl. In some embodiments, Ring F is phenyl or pyridinyl. In some embodiments, Ring F is phenyl.

In some embodiments, Ring F is phenyl and R²² is has the following structure:

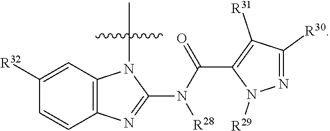

In some embodiments, Ring F is pyridine and R²² is has one of the following structures:

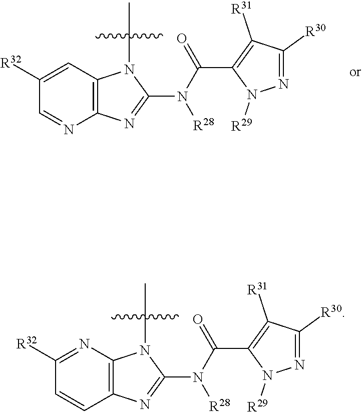

In an embodiment, R²² is has the following structure:

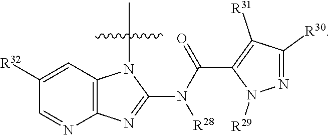

In some embodiments, Ring F is substituted with one R³² and substituted with one additional substituent selected from OC₁₋₆alkyleneNR³³R³⁴, OC₂₋₆alkenyleneNR³³R³⁴, OC₂₋₆alkynyleneNR³³R³⁴, OC₁₋₆alkyleneNR³⁵C₂₋₆alkenyleneOC₁₋₆alkyl, OC₁₋₆alkyleneNR³⁵1-6alkyleneOC₁₋₆alkyl, OC₂₋₆alkenyleneNR³⁵C₁₋₆alkyleneOC₁₋₆alkyl, OC₂₋₆alkynyleneNR³⁵C₁₋₆alkyleneOC₁₋₆alkyl, OC₁₋₆alkyleneNR³⁵C₂₋₆alkenyleneOC₁₋₆alkyl, OC₂₋₆alkenyleneNR³⁵C₂₋₆alkenyleneOC₁₋₆alkyl, OC₂₋₆alkynyleneNR³⁵C₂₋₆alkenyleneOC₁₋₆alkyl, OC₁₋₆alkyleneNR³⁵C₂₋₆alkynyleneOC₁₋₆alkyl, OC₂₋₆alkenyleneNR³⁵C₂₋₆alkynyleneOC₁₋₆alkyl, and OC₂₋₆alkynyleneNR³⁵C₂₋₆alkynyleneOC₁₋₆alkyl and each alkyl, alkenyl, alkynyl, alkylene, alkenylene and alkynylene group is optionally fluoro-substituted.

In some embodiments, Ring F is substituted with one R³² and substituted with one additional substituent selected from OC₁₋₄alkyleneNR³³R³⁴, OC₂₋₄alkenyleneNR³³R³⁴, OC₂₋₄alkynyleneNR³³R³⁴, OC₁₋₄alkyleneNR³⁵C₁₋₄alkyleneOC₁₋₄alkyl, OC₂₋₄alkenyleneNR³⁵C₁₋₄alkyleneOC₁₋₄alkyl, OC₂₋₄alkynyleneNR³⁵C₁₋₄alkyleneOC₁₋₄alkyl, OC₁₋₄alkyleneNR³⁵C₂₋₄alkenyleneOC₁₋₄alkyl, OC₂₋₄alkenyleneNR³⁵C₂₋₄alkenyleneOC₁₋₆alkyl, OC₂₋₄alkynyleneNR²¹C₂₋₄alkenyleneOC₁₋₄alkyl, OC₁₋₄alkyleneNR³⁵C₂₋₄alkynyleneOC₁₋₄alkyl, OC₂₋₄alkenyleneNR³⁵C₂₋₄alkynyleneOC₁₋₄alkyl, and OC₂₋₄alkynyleneNR³⁵C₂₋₄alkynyleneOC₁₋₄alkyl, and each alkyl, alkenyl, alkynyl, alkylene, alkenylene and alkynylene group is optionally fluoro-substituted. In some embodiments, Ring F is substituted with one R³² and substituted with one additional substituent selected from OC₁₋₄alkyleneNR³³R³⁴ and OC₁₋₄alkyleneNR³⁵C₁₋₄alkyleneOC₁₋₄alkyl, and each alkyl, and alkylene group is optionally fluoro-substituted.

In some embodiments, Ring F is substituted with one R³² and substituted with OC₁₋₄alkyleneNR³³R³⁴, and each alkylene group is optionally fluoro-substituted. In some embodiments, Ring F is substituted with one R³² and substituted with one additional substituent selected from OCH₂NR³³R³⁴, OCH₂CH₂NR³³R³⁴ and OCH₂CH₂CH₂NR³³R³⁴

In some embodiments, Ring F is substituted with one additional substituent selected from C₁₋₄alkyleneNR³³R³⁴ and OC₁₋₄alkyleneNR³³R³⁴ wherein R³³ and R³⁴ are joined to form, together with the atoms therebetween, a 3-6 membered saturated or unsaturated ring, optionally containing one additional heteroatom selected from NR³⁸, O S, SO and SO₂, and each alkylene group is optionally fluoro-substituted. In some embodiments, Ring F is substituted with one additional substituent selected from C₁₋₄alkyleneNR³³R³⁴ and OC₁₋₄alkyleneNR³³R³⁴, wherein R³³ and R³⁴ are joined to form, together with the atoms therebetween, a 3 or 4 membered saturated or unsaturated ring, optionally containing one additional heteroatom selected from NR³⁸, O, S, SO and SO₂, and each alkylene group is optionally fluoro-substituted. In some embodiments, R³³ and R³⁴ are joined to form, together with the atoms therebetween, aziranyl, azetidinyl or diazetidinyl. In some embodiments, R³³ and R³⁴ are joined to form, together with the nitrogen therebetween, a 5 or 6 membered saturated or unsaturated ring selected from pyrrolidinyl, imidazolidinyl, pyrazolidinyl, thiazolidinyl, isothiazolidinyl, piperidinyl, piperazinyl, thiomorpholinyl, and morpholinyl. In some embodiments, R³³ and R³⁴ are joined to form, together with the nitrogen therebetween, piperidinyl, piperazinyl, or morpholinyl. In some embodiments, R³³ and R³⁴ are joined to form, together with the nitrogen therebetween, piperazinyl or morpholinyl. In some embodiments, R³³ and R³⁴ are joined to form, together with the nitrogen therebetween, pyrrolidinyl.

In some embodiments, R³³ and R³⁴ are joined to form, together with the nitrogen therebetween, a 7-10 membered saturated or unsaturated ring, optionally containing one additional heteroatom selected from NR³⁸, O, S, SO and SO₂. In some embodiments, R³³ and R³⁴ are joined to form, together with the nitrogen therebetween, a 7-10 membered saturated or unsaturated ring selected from azepanyl, axepinyl, diazepinyl, oxazepanyl, thiazepinyl, azocanyl, azocinyl, azonanyl and azocinyl. In some embodiments, R³³ and R³⁴ are joined to form, together with the nitrogen therebetween, azepanyl, axepinyl, diazepinyl, or oxazepanyl. In some embodiments, R³³ and R³⁴ are joined to form, together with the nitrogen therebetween, oxazepanyl.

In some embodiments, R³⁸ is selected from H, D, CHO, CO₂H, CONH₂, C₁₋₄alkyl, C₁₋₄alkenyl, C₁₋₄alkynyl, C(O)C₁₋₆alkyl, CO₂C₁₋₄alkyl, OC₁₋₄alkyl, CONHC₁₋₄alkyl, CON(C₁₋₄alkyl)(C₁₋₄alkyl), C₁₋₄alkyleneOC₁₋₄alkyl, C₂₋₄alkenyleneOC₁₋₄alkyl, and C₂₋₄alkynyleneOC₁₋₄alkyl, and each alkyl, alkenyl, alkynyl, alkylene, alkenylene and alkynylene group is optionally fluoro-substituted.

In some embodiments, R³⁸ is selected from H, D, C₁₋₄alkyl, C₁₋₄alkenyl, C₁₋₄alkynyl, C(O)C₁₋₄alkyl, CO₂C₁₋₄alkyl, CONHC₁₋₄alkyl, C₁₋₄alkyleneOC₁₋₄alkyl, and each alkyl, alkenyl, alkynyl and alkylene group is optionally fluoro-substituted. In some embodiments, R³⁸ is selected from H, D, C₁₋₄alkyl, and C(O)C₁₋₄alkyl, and each alkyl group is optionally fluoro-substituted. In some embodiments, R³⁸ is selected from H, D, CH₃, CF₃, CHF₂, C(O)CH₃, C(O)CF₃, and C(O)CHF₂. In some embodiments, R³⁸ is selected from H, D, CH₃, CF₃, and CHF₂. In some embodiments, R³⁸ is selected from C(O)CH₃, C(O)CF₃, and C(O)CHF₂. In some embodiments, R³⁸ is C(O)CH₃.

In some embodiments, R³³ and R³⁴ are joined to form, together with the nitrogen therebetween, a 7-10 membered saturated or unsaturated ring, optionally containing one additional heteroatom selected from NR³⁸, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl, suitably one or more of D, CHO, CO₂H, CONH₂, C₁₋₆alkyl, C(O)C₁₋₆alkyl, CO₂C₁₋₆alkyl, OC₁₋₆alkyl, C₁₋₆alkyleneOC₁₋₆alkyl, C₂₋₆alkenyleneOC₁₋₆alkyl, and C₂₋₆alkynyleneOC₁₋₆alkyl more suitably one or more of D, CHO, C₁₋₆alkyl, C(O)C₁₋₆alkyl, OC₁₋₆alkyl, or C₁₋₆alkyleneOC₁₋₆alkyl, and each alkyl, alkenyl, alkynyl, alkylene, alkenylene and alkynylene group is optionally fluoro-substituted.

In some embodiments, R³³ and R³⁴ are joined to form, together with the nitrogen therebetween, a 3-6 membered saturated or unsaturated ring, optionally containing one additional heteroatom selected from NR³⁸, O, S, SO and SO₂, and substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl, suitably one or more of D, CHO, CO₂H, CONH₂, C₁₋₆alkyl, C(O)C₁₋₆alkyl, CO₂C₁₋₆alkyl, OC₁₋₆alkyl, C₁₋₆alkyleneOC₁₋₆alkyl, C₂₋₆alkenyleneOC₁₋₆alkyl, and C₂₋₆alkynyleneOC₁₋₆alkyl, more suitably one or more of D, CHO, C₁₋₆alkyl, C(O)C₁₋₆alkyl, OC₁₋₆alkyl, or C₁₋₆alkyleneOC₁₋₆alkyl, and each alkyl, alkenyl, alkynyl, alkylene, alkenylene and alkynylene group is optionally fluoro-substituted.

In some embodiments, R³³ and R³⁴ are joined to form, together with the nitrogen therebetween, a 5 or 6 membered saturated or unsaturated ring, optionally containing one additional heteroatom selected from NR³⁸, O, S, SO and SO₂, and substituted with one or more of D. In some embodiments, R³³ and R³⁴ are joined to form, together with the nitrogen therebetween, deutero-substituted piperidinyl, piperazinyl, or morpholinyl. In some embodiments, R³³ and R³⁴ are joined to form, together with the nitrogen therebetween, deutero-substituted morpholinyl. In some embodiments, R³³ and R³⁴ are joined to form, together with the nitrogen therebetween, a 5 or 6 membered saturated or unsaturated ring that is substituted with C(O)C₁₋₆alkyl or C(O)C₁₋₆fluoroalkyl, suitably C(O)C₁₋₄alkyl or C(O)C₁₋₄fluoroalkyl, more suitably C(O)CH₃, C(O)CHF₂, or C(O)CHF₃, even more suitably C(O)CH₃. In some embodiments, R³³ and R³⁴ are joined to form, together with the nitrogen therebetween, a 5 or 6 membered saturated or unsaturated ring, optionally containing one additional heteroatom selected from NR³⁸, O, S, SO and SO₂, and substituted with C₁₋₆alkyleneOC₁₋₆alkyl, suitably C₁₋₄alkyleneOC₁₋₄alkyl which is optionally fluoro-substituted, more suitably CH₂OCH₃, CH₂OCF₃, CH₂OCF₂H, CH₂CH₂OCH₃, CH₂CH₂OCF₂H or CH₂CH₂OCF₃, even more suitably CH₂OCH₃, CH₂OCF₃ or CH₂OCF₂H, most suitably CH₂OCH₃ or CH₂OCF₂H.

In some embodiments, R³³ and R³⁴ are joined to form, together with the atoms therebetween, a 3-10 membered saturated or unsaturated ring, containing one additional heteroatom selected from NR³⁸, O, and S. In some embodiments, R³³ and R³⁴ are joined to form, together with the atoms therebetween, a 3-10 membered saturated or unsaturated ring, containing one additional heteroatom selected from S, SO and SO₂. In some embodiments, R³³ and R³⁴ are joined to form, together with the atoms therebetween, a 3-10 membered saturated or unsaturated ring, containing one additional heteroatom selected from NR³⁸ and O.

In some embodiments, Ring F is substituted with one R³² and substituted with OC₁₋₄alkyleneNR³⁵C₁₋₄alkyleneOC₁₋₄alkyl wherein R³⁵ is selected from H and C₁₋₄alkyl, and each alkyl, and alkylene group is optionally fluoro-substituted. In some embodiments, Ring F is substituted with one R³² and substituted with one additional substituent selected from OCH₂CH₂CH₂NHCH₂CH₂OCH₃, OCH₂CH₂CH₂N(CH₃)CH₂CH₂OCH₃, OCH₂CH₂CH₂NHCH₂CH₂OCF₃, OCH₂CH₂CH₂N(CH₃)CH₂CH₂OCF₃, OCH₂CH₂CH₂NHCH₂CH₂OCHF₂, and OCH₂CH₂CH₂N(CH₃)CH₂CH₂OCHF₂. In some embodiments, Ring F is substituted with one R³² and substituted with one additional substituent selected from OCH₂CH₂CH₂NHCH₂CH₂OCH₃, OCH₂CH₂CH₂N(CH₃)CH₂CH₂OCH₃, OCH₂CH₂CH₂NHCH₂CH₂OCHF₂, and OCH₂CH₂CH₂N(CH₃)CH₂CH₂OCHF₂.

In some embodiments, R³² is selected from COR³⁶, CO₂R³⁶ and CONR³⁶R³⁷; and R³⁶ and R³⁷ are independently selected from H, C₁₋₄alkyl and C₁₋₄fluoroalkyl. In some embodiments, R³² is selected from COR³⁶, CO₂R³⁶ and CONR³⁶R³⁷; and R³⁶ and R³⁷ are independently selected from H and CH₃. In some embodiments, R³² is CONR³⁶R³⁷; and R³⁶ and R³⁷ are independently selected from H and CH₃. In an embodiment, R³² is CONH₂.

In some embodiments, R¹⁴ is selected from H, C₁₋₄alkyl and C₁₋₄fluoroalkyl. In an embodiment, R¹⁴ is H.

In some embodiments, R²⁸ is selected from H, C₁₋₄alkyl and C₁₋₄fluoroalkyl. In an embodiment, R²⁸ is H.

In some embodiments, R¹⁵, R¹⁶ and R¹⁷ are independently selected from H, C₁₋₆alkyl and C₁₋₆fluoroalkyl. In some embodiments, R¹⁵ and R¹⁶ are independently selected from H, C₁₋₆alkyl, and C₁₋₆fluoroalkyl and R¹⁷ is H. In some embodiments, R¹⁵ and R¹⁶ are independently selected from CH₃ and CH₂CH₃. In an embodiment, R¹⁵ is CH₂CH₃. In an embodiment, R¹⁶ is CH₃.

In some embodiments, R²⁹, R³⁰ and R³¹ are independently selected from H and C₁₋₆alkyl. In some embodiments, R²⁹ and R³⁰ are independently selected from H and C₁₋₆alkyl and R³¹ is H. In some embodiments, R²⁹ and R³⁰ are independently selected from CH₃ and CH₂CH₃. In an embodiment, R²⁹ is CH₂CH₃. In an embodiment, R³⁰ is CH₃.

In some embodiments, the compound of Formula II is selected from the compounds listed below:

| Compound ID | Structure |
|---|---|
| U | 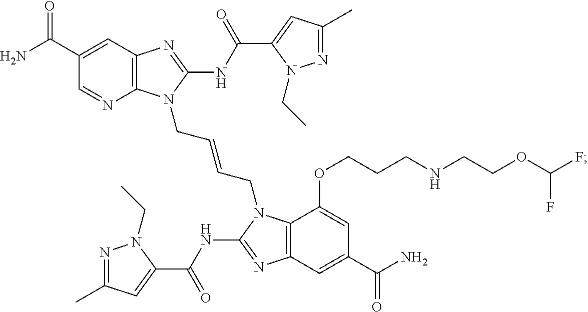 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNCCOC(F)F)c21] |
| V | 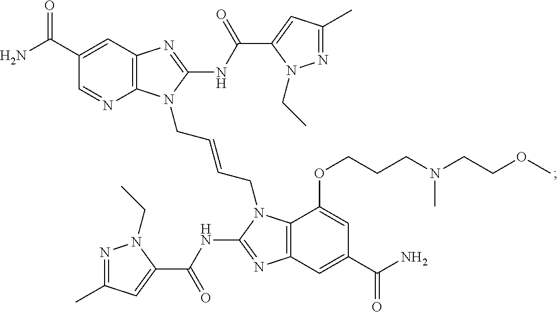 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN(C)CCOC)c21] |
| W | 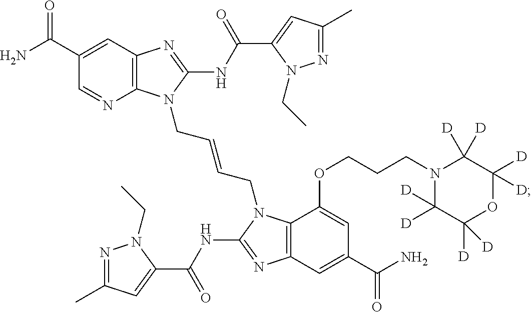 |
| X | 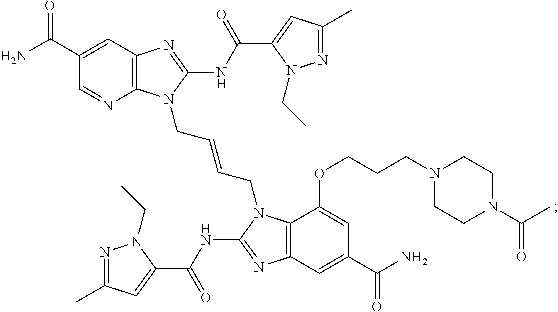 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCN(C(C)=O)CC3)c21] |
| Y | 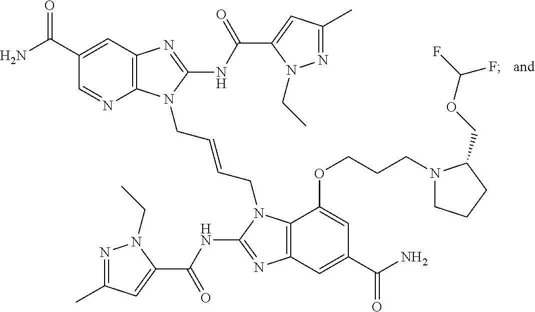 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC[C@H]3COC(F)F)c21] |
| Z | 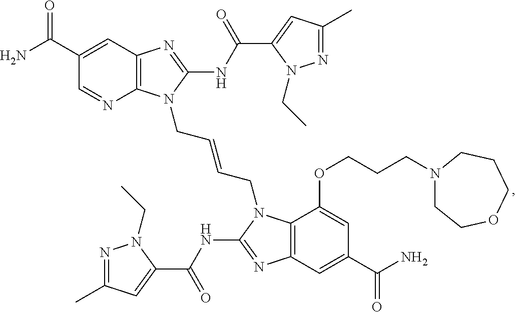 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCCOCC3)c21] |

or a pharmaceutically acceptable salt, solvate and/or prodrug thereof.

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, Pa., 1980, pp. 1530-1533 for further discussion of suppository dosage forms.

In some embodiments, compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In some embodiments, one or more compounds of the application may also be coupled to suitable antibodies as targetable drug carriers. For example, compounds of application may be coupled to suitable antibodies to form an antibody-drug conjugate (ADC), as described for example in Polakis, P., Pharmacol. Revs., 2016, 68, 3-19. Therefore the present application includes an ADC comprising one or more compounds of the application. An antibody may generally include any polypeptide comprising a framework region from an immunoglobulin or fragments thereof that specifically binds and recognizes an antigen, such as a carbohydrate, polynucleotide, lipid, polypeptide, etc. In some embodiments, the antibody is one that specifically binds to a cancer antigen, for example known cancer antigens CD22, CD33, CD30, HER2, Mesothelin, Melan-A, CD19, CD20, CD79b, Trop2, HER3, MAGE, MART-1. In some embodiments, the antibody is one that specifically binds to an immune cell surface receptor, for example CD80, CD86, GMCSF-R, DC-SIGN, CD36. In some embodiments, the antibody is one that specifically binds to an abnormally expressed protein characteristic of a cancer. In some embodiments, compounds of application may be coupled to suitable antibodies to form an ADC through a linker. The linker may be or comprise a cleavable group that allows the compound of the application to be cleaved from the rest of the conjugate in vivo by the biological environment. In some embodiments, the linker is cleaved in or near the desired target site of action. In some embodiments, the linker is noncleavable.

In some embodiments, compounds of the application may be coupled with suitable viral, non-viral or other vectors. Viral vectors may include retrovirus, lentivirus, adenovirus, herpesvirus, poxvirus, alphavirus, vaccinia virus and/or adeno-associated viruses. Non-viral vectors may include nanoparticles, cationic lipids, cationic polymers, metallic nanoparticles, nanorods, liposomes, micelles, microbubbles, cell-penetrating peptides, and/or lipospheres. Nanoparticles may include silica, lipid, carbohydrate, and/or other pharmaceutically acceptable polymers. The compounds of the application including pharmaceutically acceptable salts, solvates and prodrugs thereof are suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of a pharmaceutically acceptable carrier, all percentages by weight being based on the total composition.

Compounds of the application may be used alone or in combination with other known agents useful for treating diseases, disorders or conditions mediated by STING protein activation, or that are treatable by activation of STING. When used in combination with other agents useful in treating diseases, disorders or conditions mediated by STING protein activation, or that are treatable by activation of STING, it is an embodiment that the compounds of the application are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g. a compound of Formula I), an additional therapeutic agent, and a pharmaceutically acceptable carrier.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg.

In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three or four daily doses.

To be clear, in the above, the term “a compound” also includes embodiments wherein one or more compounds are referenced.

### III. Methods and Uses of the Application

The compounds of the application have been shown to be capable of activating STING activity, such as STING protein activity.

Accordingly, the present application includes a method for activating STING in a cell, either in a biological sample or in a patient, comprising administering an effective amount of one or more compounds of the application to the cell. The application also includes a use of one or more compounds of the application for activating STING in a cell as well as a use of one or more compounds of the application for the preparation of a medicament for activating STING in a cell. The application further includes one or more compounds of the application for use in activating STING in a cell.

As the compounds of the application have been shown to be capable of activating STING protein activity, the compounds of the application are useful for treating diseases, disorders or conditions by activating STING. Therefore the compounds of the present application are useful as medicaments. Accordingly, the present application includes a compound of the application for use as a medicament.

The present application also includes a method of treating a disease, disorder or condition by activation of STING comprising administering a therapeutically effective amount of one or more compounds of the application to a subject in need thereof.

The present application also includes a use of one or more compounds of the application for treatment of a disease, disorder or condition by activation of STING as well as a use of one or more compounds of the application for the preparation of a medicament for treatment of a disease, disorder or condition by activation of STING. The application further includes one or more compounds of the application for use in treating a disease, disorder or condition by activation of STING.

In an embodiment, the disease, disorder or condition is a neoplastic disorder. Accordingly, the present application also includes a method of treating a neoplastic disorder comprising administering a therapeutically effective amount of one or more compounds of the application to a subject in need thereof. The present application also includes a use of one or more compounds of the application for treatment of a neoplastic disorder as well as a use of one or more compounds of the application for the preparation of a medicament for treatment of a neoplastic disorder. The application further includes one or more compounds of the application for use in treating a neoplastic disorder. In an embodiment, the treatment is in an amount effective to ameliorate at least one symptom of the neoplastic disorder, for example, reduced cell proliferation or reduced tumor mass, among others, in a subject in need of such treatment.

Compounds of the application have been demonstrated to be effective against a panel of human tumor cell line. Therefore in another embodiment of the present application, the disease, disorder or condition that is treated by activation of STING is cancer. Accordingly, the present application also includes a method of treating cancer comprising administering a therapeutically effective amount of one or more compounds of the application to a subject in need thereof. The present application also includes a use of one or more compounds of the application for treatment of cancer as well as a use of one or more compounds of the application for the preparation of a medicament for treatment of cancer. The application further includes one or more compounds of the application for use in treating cancer. In an embodiment, the compound is administered for the prevention of cancer in a subject such as a mammal having a predisposition for cancer.

In an embodiment, the cancer is selected from a cancer of the skin, blood, prostate, colorectum, pancreas, kidney, ovary, breast, for example mammary, liver, tongue and lung. In another embodiment, the cancer is selected from leukaemia, lymphoma, non-Hodgkin's lymphoma and multiple myeloma. In a further embodiment of the present application, the cancer is selected from leukemia, melanoma, lung cancer, colon cancer, brain cancer, ovarian cancer, breast cancer, prostate cancer and kidney cancer.

In an embodiment, the disease, disorder or condition that is treated by activation of STING is a disease, disorder or condition associated with an uncontrolled and/or abnormal cellular activity affected directly or indirectly by activation of STING. In another embodiment, the uncontrolled and/or abnormal cellular activity that is affected directly or indirectly by activation of STING is proliferative activity in a cell. Accordingly, the application also includes a method of inhibiting proliferative activity in a cell, comprising administering an effective amount of one or more compounds of the application to the cell. The present application also includes a use of one or more compounds of the application for inhibition of proliferative activity in a cell as well as a use of one or more compounds of the application for the preparation of a medicament for inhibition of proliferative activity in a cell. The application further includes one or more compounds of the application for use in inhibiting proliferative activity in a cell.

The present application also includes a method of inhibiting uncontrolled and/or abnormal cellular activities affected directly or indirectly by STING protein in a cell, either in a biological sample or in a subject, comprising administering an effective amount of one or more compounds of the application to the cell. The application also includes a use of one or more compounds of the application for inhibition of uncontrolled and/or abnormal cellular activities affected directly or indirectly by STING protein in a cell as well as a use of one or more compounds of the application for the preparation of a medicament for inhibition of uncontrolled and/or abnormal cellular activities affected directly or indirectly by STING protein in a cell. The application further includes one or more compounds of the application for use in inhibiting uncontrolled and/or abnormal cellular activities affected directly or indirectly by STING protein in a cell.

Accordingly, the present application also includes a method of treating a disease, disorder or condition that is treatable by activation of STING comprising administering a therapeutically effective amount of one or more compounds of the application in combination with another known agent useful for treatment of a disease, disorder or condition treatable by activation of STING to a subject in need thereof. The present application also includes a use of one or more compounds of the application in combination with another known agent useful for treatment of a disease, disorder or condition treatable by activation of STING for treatment of a disease, disorder or condition treatable by activation of STING, as well as a use of one or more compounds of the application in combination with another known agent useful for treatment of a disease, disorder or condition treatable by activation of STING for the preparation of a medicament for treatment of a disease, disorder or condition treatable by activation of STING. The application further includes one or more compounds of the application in combination with another known agent useful for treatment of a disease, disorder or condition treatable by activation of STING for use in treating a disease, disorder or condition treatable by activation of STING. In an embodiment, the disease, disorder or condition treatable by activation of STING is cancer such as acute myloid leukemia (AML), acute lyphocytic leukemia (ALL), melanoma, prostate cancer, endometrial cancer, schwannoma, mantel cell lymphoma, rhabdomyosarcoma, glioma, glioblastoma, B-ALL, T-ALL, lung cancer, gastric cancer, pancreatic cancer and breast cancer.

In a further embodiment, the disease, disorder or condition treatable by activation of STING is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies.

In some embodiments, the subject is a mammal. In some embodiments, the subject is human.

### IV. Preparation of Compounds of the Application

Compounds of the present application can be prepared by various synthetic processes. The choice of particular structural features and/or substituents may influence the selection of one process over another. The selection of a particular process to prepare a given compound of Formula I is within the purview of the person of skill in the art. Some starting materials for preparing compounds of the present application are available from commercial chemical sources. Other starting materials, for example as described below, are readily prepared from available precursors using straightforward transformations that are well known in the art. In the Schemes below showing the preparation of compounds of the application, all variables are as defined in Formula I, unless otherwise stated.

In some embodiments, compounds of Formula I are prepared as shown in Scheme 1. Therefore, amino compounds of Formula III are coupled with carboxylic acid compounds of Formula IV under amide formation conditions. For example, the amino compounds of Formula III can be coupled with the carboxylic acid compounds of Formula IV in the presence of coupling reagents such as hexafluorophosphate benzotriazole tetramethyl uronium (HBTU) in an inert solvent to provide compounds of Formula I.

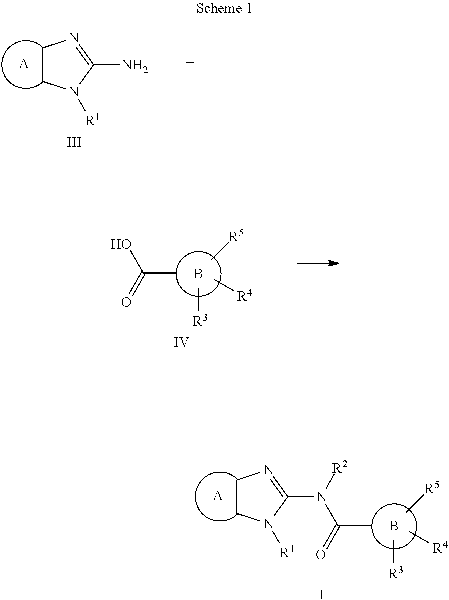

In some embodiments, the compounds of Formula III are prepared as shown in Scheme 2. Therefore compounds of Formula VI are reacted with various activated nitriles, such as BrCN under ring formation conditions to provide the compound of Formula III.

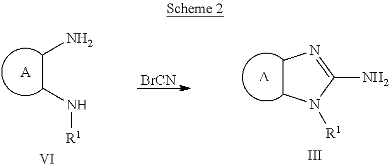

In some embodiments, compounds of Formula I wherein R² is H are prepared as shown in Scheme 3. Therefore compounds of Formula VI are coupled with various isothiocyanates of Formula VII under ring formation conditions, for example, in the presence of 1-ethyl-3-(3-dimethylaminopropyl) carbodiimide (EDCl) to provide compounds of Formula I wherein R² is H.

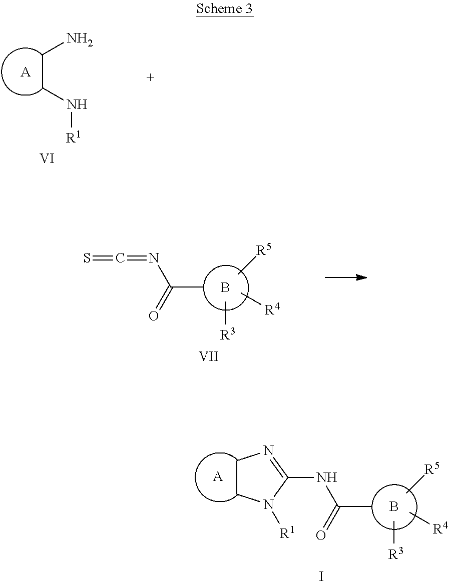

In some embodiments, compounds of Formula VI are prepared as shown in Scheme 4. Therefore halo-nitro compounds of Formula VIII are coupled to various amines IX by displacement using various bases such as DIPEA. The resulting nitro compounds XI can be reduced using reducing agents such as sodium hydrosulfite (Na₂S₂O₄) or Zinc dust and ammonium chloride to provide compounds of Formula VI.

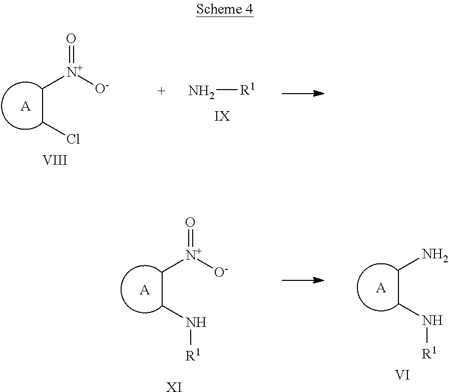

In some embodiments, when R⁷ in compounds of Formula I is

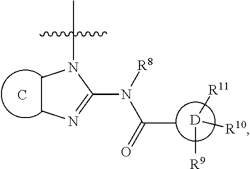

R⁷ is prepared as shown in Schemes 1 to 4.

In some embodiments, when R⁷ in compounds of Formula I is

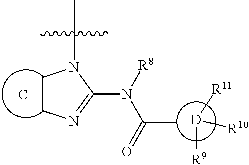

and is identical to

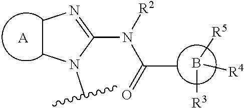

(i.e., compound of Formula I is a dimer) then the compound of Formula I can be prepared using simultaneous imidazole ring formation reactions using, for example, a compound of Formula XII.

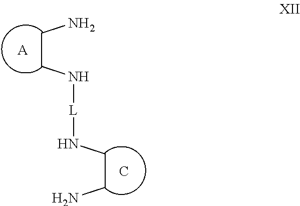

wherein ring A and ring C are as defined in Formula I and L is selected from C₁₋₁₀alkylene, C₂₋₁₀alkenylene and C₂₋₁₀alkynylene, and, for example, the methods shown in Scheme 2 or Scheme 3 followed by reaction with a compound of Formula IV as shown in Scheme 1.

The compounds of Formula IV VI, VII and VIII may be prepared using methods known in the art or are commercially available.

Compounds of Formula II, being a subset of compounds of Formula I, may be prepared using analogous methods to those shown above for the preparation of compounds of Formula I.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine).

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

The following non-limiting examples are illustrative of the present application:

### EXAMPLES

### General Methods

All starting materials used herein were commercially available or earlier described in the literature. The ¹H and ¹³C NMR spectra were recorded either on Bruker 300, Bruker DPX400 or Varian +400 spectrometers operating at 300, 400 and 400 MHz for 1H NMR respectively, using TMS or the residual solvent signal as an internal reference, in deuterated chloroform as solvent unless otherwise indicated. All reported chemical shifts are in ppm on the delta-scale, and the fine splitting of the signals as appearing in the recordings is generally indicated, for example as s: singlet, br s: broad singlet, d: doublet, t: triplet, q: quartet, m: multiplet. Unless otherwise indicated, in the tables below, ¹H NMR data was obtained at 400 MHz, using CDCl₃ as the solvent.

Purification of products was carried out using Chem Elut Extraction Columns (Varian, cat #1219-8002), Mega BE-SI (Bond Elut Silica) SPE Columns (Varian, cat #12256018; 12256026; 12256034) or by flash chromatography in silica-filled glass columns.

### Example 1 Preparation of Compound G

### (a) 6-chloro-5-nitro-pyridine-3-carboxamide

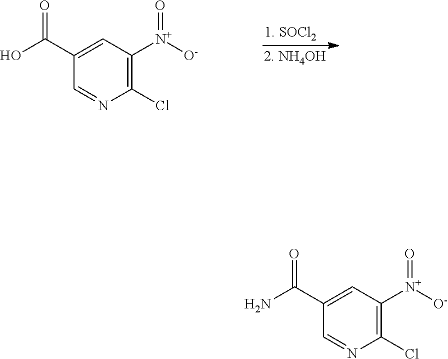 [SMILES: O=C(O)c1cnc(Cl)c([N+](=O)[O-])c1 ; NC(=O)c1cnc(Cl)c([N+](=O)[O-])c1]

6-chloro-5-nitro-pyridine-3-carboxylic acid (4.05 g, 20 mmol) was stirred in SOCl₂ (20 mL) at reflux for 4 h. The mixture was concentrated in vacuo and dried under vacuum then stirred in THF (20 mL) at −78° C. and treated with concentrated aqueous ammonium hydroxide (14 mL). The mixture was stirred for 10 min then warmed to room temperature and quenched with water; the solid material was collected via vacuum filtration to give the desired product as an off-white solid (3.16 g, 78%). [MH]⁺=202.0

### (b) 6-(4-aminobutylamino)-5-nitro-pyridine-3-carboxamide

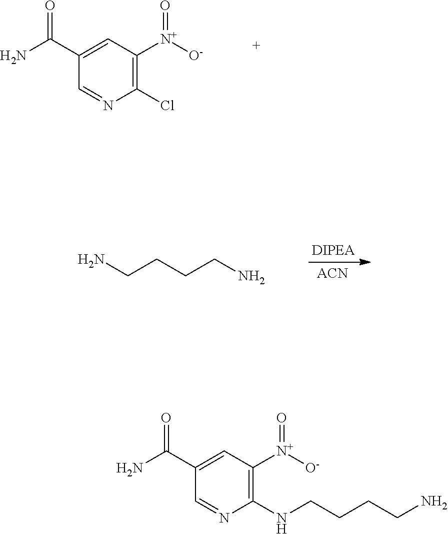

To a stirred solution of butane-1,4-diamine (582 mg, 6.0 mmol) in ACN (8 mL) and DIPEA (1.05 mL, 6.0 mmol) was added 6-chloro-5-nitro-pyridine-3-carboxamide (400 mg, 2.0 mmol), portionwise over 1 h; stirred at RT overnight. The mixture was diluted with water and ether, then filtered to collect the desired product as a light yellow solid (370 mg, 73%). ¹H NMR (d₆-DMSO) δ 8.92 (d, J=4 Hz, 1H), 8.88 (d, J=4 Hz, 1H), 8.09 (s, 1H), 7.45 (s, 1H), 3.62 (t, J=8 Hz, 2H), 3.31 (brs, 2H), 2.61-2.53 (m, 2H), 1.69-1.57 (m, 2H), 1.46-1.33 (m, 2H).

### (c) benzyl N-[4-[(5-carbamoyl-3-nitro-2-pyridyl)amino]butyl]carbamate

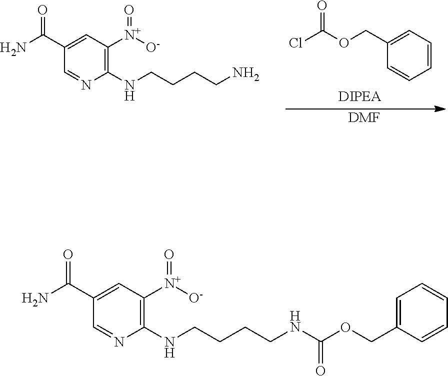

To a stirred solution of 6-(4-aminobutylamino)-5-nitro-pyridine-3-carboxamide (360 mg, 1.4 mmol) in DMF (6 mL) and DIPEA (488 μL, 2.8 mmol) at RT was added benzyl chloroformate (260 mL, 1.8 mmol), dropwise. The mixture was diluted with water and filtered; the filter cake was washed with water and diethyl ether then dried in the oven overnight giving the desired product as a yellow solid (480 mg, 89%). ¹H NMR (d₆-DMSO) δ 8.93 (d, J=4 Hz, 1H), 8.88 (d, J=4 Hz, 1H), 8.79 (t, J=6 Hz, 1H), 8.09 (brs, 1H), 7.46 (brs, 1H), 7.39-7.23 (m, 6H), 5.01 (s, 2H), 3.62 (q, J=8 Hz, 2H), 3.03 (q, J=8 Hz, 2H), 1.67-1.57 (m, 2H), 1.52-1.42 (m, 2H).

### (d) benzyl N-[4-[(3-amino-5-carbamoyl-2-pyridyl)amino]butyl]carbamate

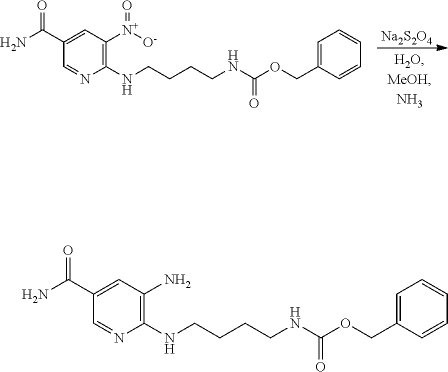 [SMILES: NC(=O)c1cnc(NCCCCNC(=O)OCc2ccccc2)c([N+](=O)[O-])c1 ; "Na2S2O4" ; NC(=O)c1cnc(NCCCCNC(=O)OCc2ccccc2)c(N)c1]

To a stirred suspension of benzyl N-[4-[(5-carbamoyl-3-nitro-2-pyridyl)amino]butyl]carbamate (480 mg, 1.2 mmol) in methanol (15 mL) and ammonium hydroxide (2.03 mL, 30 mmol) was added sodium dithionite (1.29 g, 7.4 mmol) as a solution in water (5 mL); mixture was stirred at room temperature for 4 h. The mixture was then diluted with water and extracted with ethyl acetate (3×). The combined organics were washed with brine (1×) and dried over anhydrous sodium sulfate; filtered and concentrated to give crude product as a light brown foam, used directly in the subsequent reaction. [MH]⁺=358.2

### (e) benzyl N-[4-(2-amino-6-carbamoyl-imidazo[4,5-b]pyridin-3-yl)butyl]carbamate

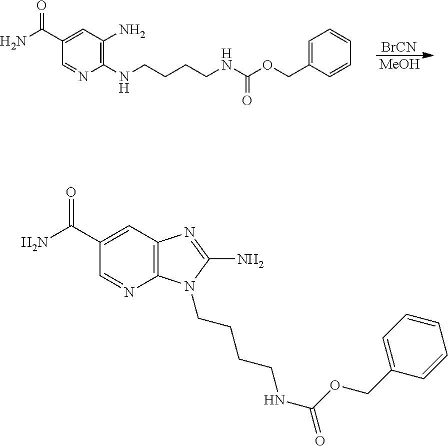

To a stirred solution of benzyl N-[4-[(3-amino-5-carbamoyl-2-pyridyl)amino]butyl]carbamate (1.2 mmol) in methanol was added cyanogen bromide (190 mg, 1.8 mmol); mixture was stirred at 60° C. overnight; the mixture was treated with more cyanogen bromide (190 mg, 1.8 mmol) and stirred overnight at 60° C. The mixture was treated with 2 M ammonia in methanol, concentrated onto silica gel then chromatographed in 0 to 12% (2M NH₃ in MeOH) in dichloromethane. Product containing fractions were concentrated in vacuo giving the desired product as a rose coloured solid. [MH]=383.2

### (f) benzyl N-[4-[6-carbamoyl-2-[(2-ethyl-5-methyl-pyrazole-3-carbonyl)amino]imidazo[4,5-b]pyridin-3-yl]butyl]carbamate (Compound G)

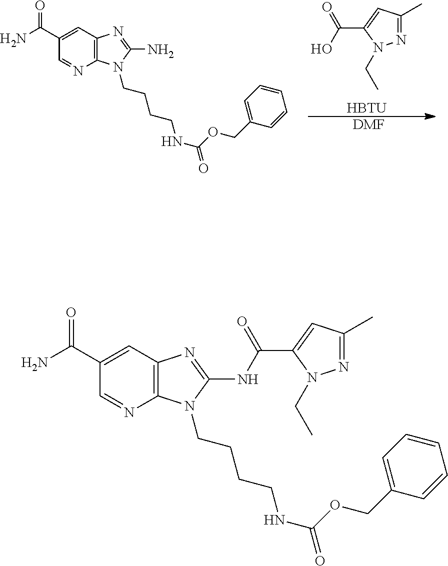

To a stirred solution of 2-ethyl-5-methyl-pyrazole-3-carboxylic acid (60 mg, 0.39 mmol) and HBTU (148 mg, 0.39 mmol) in DMF (3 mL) was added DIPEA (136 μL, 0.78 mmol). The resulting mixture as stirred for 10 min then treated with benzyl N-[4-(2-amino-6-carbamoyl-imidazo[4,5-b]pyridin-3-yl)butyl]carbamate (100 mg, 0.26 mmol); stirred overnight at room temperature. The mixture was diluted with ethyl acetate and washed with brine (1×), brine:water (1:1, lx) and brine (1×). The organic phase was dried, filtered and concentrated in vacuo then chromatographed in 0-7% (2 M NH₃ in MeOH) in DCM. The product containing fractions were concentrated in vacuo then triturated with diethyl ether and hexanes; filtered to collect the desired product as a beige solid (61 mg, 45%). ¹H NMR (d₆-DMSO) δ 8.78 (d, J=4 Hz, 1H), 8.17 (s, 1H), 8.15 (d, J=4 Hz, 1H), 7.55 (s, 1H), 7.37-7.21 (m, 6H), 6.72 (s, 1H), 4.98 (s, 2H), 4.62 (q, J=8 Hz, 2H), 4.27-4.17 (m, 2H), 3.11-3.00 (m, 2H), 2.19 (s, 3H), 1.90-1.79 (m, 2H), 1.52-1.41 (m, 2H), 1.37 (t, J=8 Hz, 3H).

### Example 2 Preparation of Compound H

### (a) methyl 5-[[(E)-4-(tert-butoxycarbonylamino)but-2-enyl]amino]-4-nitro-thiophene-2-carboxylate

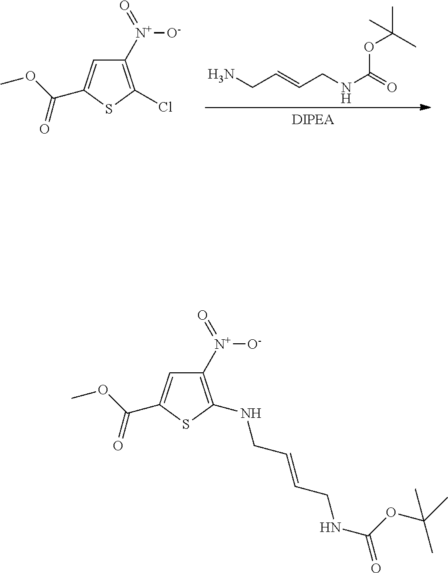 [SMILES: COC(=O)c1cc([N+](=O)[O-])c(Cl)s1 ; CC(C)(C)OC(=O)NC/C=C/CN ; COC(=O)c1cc([N+](=O)[O-])c(NC/C=C/CNC(=O)OC(C)(C)C)s1]

To a stirred solution of methyl 5-chloro-4-nitro-thiophene-2-carboxylate (1 g, 4.51 mmol) in DMF (10 mL) was added tert-butyl N-[(E)-4-aminobut-2-enyl]carbamate hydrochloride (1.51 g, 6.77 mmol) followed by DIPEA (3.14 mL, 18 mmol). The resulting red mixture was stirred at RT for 1 h, then diluted with water (slowly) giving a suspension which was filtered. The filter cake was stirred in diethyl ether and filtered to collect the solid; dried in oven to give the desired product (1.5 g, 89%). ¹H NMR (d₆-DMSO) δ 9.56 (brs, 1H), 7.86 (s, 1H), 7.01 (brs, 1H), 5.80-5.68 (m, 1H), 5.59-5.45 (m, 1H), 4.05-3.97 (m, 2H), 3.80 (s, 3H), 3.60-3.50 (m, 2H), 1.37 (s, 9H).

### (b) methyl 4-amino-5-[[(E)-4-(tert-butoxycarbonylamino)but-2-enyl]amino]thiophene-2-carboxylate

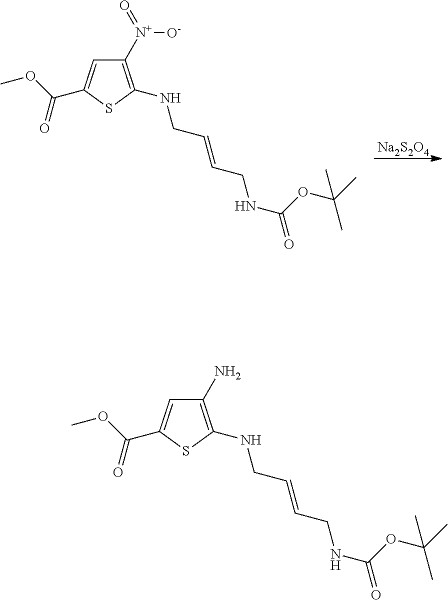

To a stirred suspension of methyl 5-[[(E)-4-(tert-butoxycarbonylamino)but-2-enyl]amino]-4-nitro-thiophene-2-carboxylate (1.5 g, 4.04 mmol) in methanol (20 mL) and ammonium hydroxide (7.2 mL, 101 mmol) was added sodium dithionite (4.2 g, 24.2 mmol) as a solution in water (20 mL); the resulting mixture was stirred at 60° C. for 2 h. The mixture was diluted with ethyl acetate and washed with brine (2×); the organic phase was dried, filtered and concentrated in vacuo giving the crude product as a red foam (1.3 g, used directly in the subsequent reaction (1.3 g, 95%). [MH]+=342.2

### (c) methyl 3-[(E)-4-(tert-butoxycarbonylamino)but-2-enyl]-2-[(2-ethyl-5-methyl-pyrazole-3-carbonyl)amino]thieno[2,3-d]imidazole-5-carboxylate

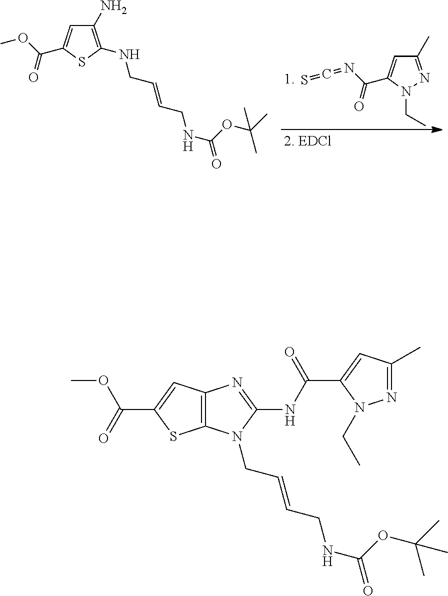 [SMILES: COC(=O)c1cc(N)c(NC/C=C/CNC(=O)OC(C)(C)C)s1 ; CCn1nc(C)cc1C(=O)N=C=S ; CCn1nc(C)cc1C(=O)Nc1nc2cc(C(=O)OC)sc2n1C/C=C/CNC(=O)OC(C)(C)C]

To a stirred solution of methyl 4-amino-5-[[(E)-4-(tert-butoxycarbonylamino)but-2-enyl]amino]thiophene-2-carboxylate (1.3 g, 3.8 mmol) in DMF (15 mL) was added 2-ethyl-5-methyl-pyrazole-3-carbonyl isothiocyanate (743 mg, 3.8 mmol). The mixture was stirred at RT for 20 min then treated with N-(3-Dimethylaminopropyl)-N′-ethylcarbodiimide hydrochloride (1.09 g, 5.7 mmol) followed by DIPEA (2 mL, 11.4 mmol); stirred at RT for 4 h. The mixture was then diluted with ethyl acetate and washed with brine (1×), brine:water (1×), saturated sodium bicarbonate (1×), brine:water (1×) and brine (1×). The organic phase was dried, filtered and concentrated in vacuo then chromatographed in 0-100% ethyl acetate in hexanes. The product containing fractions were concentrated in vacuo then stirred in methanol; the resulting suspension was filtered to collect the desired product as a white solid (1.06 g, 56%). ¹H NMR (d₆-DMSO) δ 7.73 (brs, 1H), 7.11-7.02 (m, 1H), 6.96-6.56 (m, 1H), 6.00-5.86 (m, 1H), 5.77-5.62 (m, 1H), 4.80-4.62 (m, 2H), 4.61-4.37 (m, 2H), 3.83 (s, 3H), 3.68-3.51 (m, 2H), 3.21-3.13 (m, 1H), 2.20 (s, 3H), 1.48-1.16 (s, 12H).

### (d) 3-[(E)-4-(tert-butoxycarbonylamino)but-2-enyl]-2-[(2-ethyl-5-methyl-pyrazole-3-carbonyl)amino]thieno[2,3-d]imidazole-5-carboxylic acid

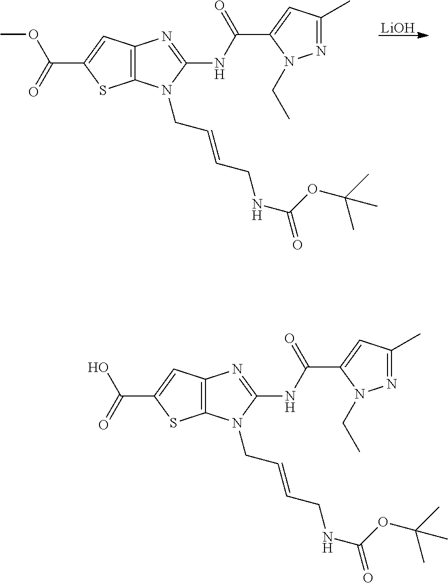

To a stirred suspension of methyl 3-[(E)-4-(tert-butoxycarbonylamino)but-2-enyl]-2-[(2-ethyl-5-methyl-pyrazole-3-carbonyl)amino]thieno[2,3-d]imidazole-5-carboxylate (1.05 g, 2.1 mmol) in methanol (10 mL) was added lithium hydroxide (200 mg, 8.36 mmol) as a solution in water. The resulting mixture was stirred at 60° C. for 2 h (a small amount of THF (1 mL) was added to aid solubility). The mixture was concentrated in vacuo, diluted with water and acidified to pH ˜3-4. The suspension was filtered to collect the product as a white solid, dried in oven (727 mg, 71%). [MH]⁺=489.2 (e) tert-butyl N-[(E)-4-[5-carbamoyl-2-[(2-ethyl-5-methyl-pyrazole-3-carbonyl)amino]thieno[2,3-d]imidazol-3-yl]but-2-enyl]carbamate (Compound H)

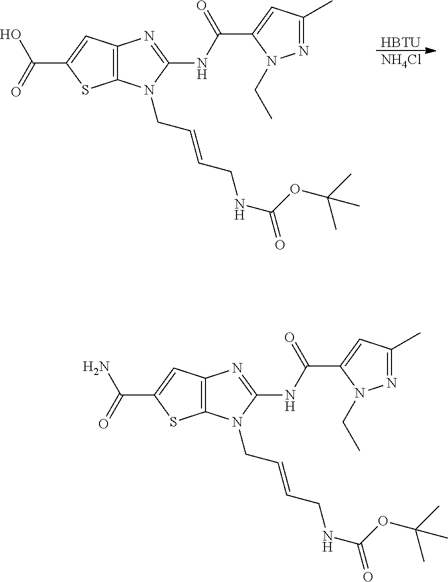

To a stirred solution of 3-[(E)-4-(tert-butoxycarbonylamino)but-2-enyl]-2-[(2-ethyl-5-methyl-pyrazole-3-carbonyl)amino]thieno[2,3-d]imidazole-5-carboxylic acid (1.81 g, 3.7 mmol) in DMF (18 mL) was added HBTU (1.69 g, 4.45 mmol) followed by DIPEA (1.61 mL, 9.25 mmol). The mixture was stirred for 15 min then treated with ammonium chloride (418 mg, 7.4 mmol), stirred for 2 h at RT. The mixture was diluted with water, filtered. The filter cake was dried, then stirred in diethyl ether and hexanes. Filtered to give the desired product (1.56 g, 87%). [MH]+=488.3

### Example 3 Preparation of Compound M

### (a) 3-[(E)-4-aminobut-2-enyl]-2-[(2-ethyl-5-methyl-pyrazole-3-carbonyl)amino]thieno[2,3-d]imidazole-5-carboxamide trihydrochloride

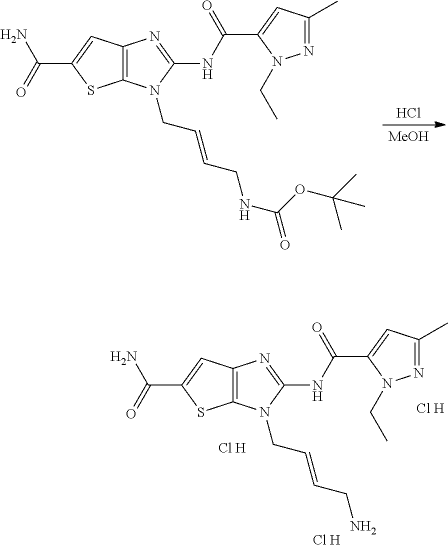

tert-butyl N-[(E)-4-[5-carbamoyl-2-[(2-ethyl-5-methyl-pyrazole-3-carbonyl)amino]thieno[2,3-d]imidazol-3-yl]but-2-enyl]carbamate (145 mg, 0.3 mmol) was stirred in methanol (1 mL) and treated with HCl, 4 M In dioxane (1.5 mL, 6 mmol). The mixture was stirred for 20 min then concentrated in vacuo giving the desired product (147 mg, quantitative yield). [MH]⁺=388.2

### (b) 3-[(E)-4-(4-carbamoyl-2-nitro-anilino)but-2-enyl]-2-[(2-ethyl-5-methyl-pyrazole-3-carbonyl)amino]thieno[2,3-d]imidazole-5-carboxamide

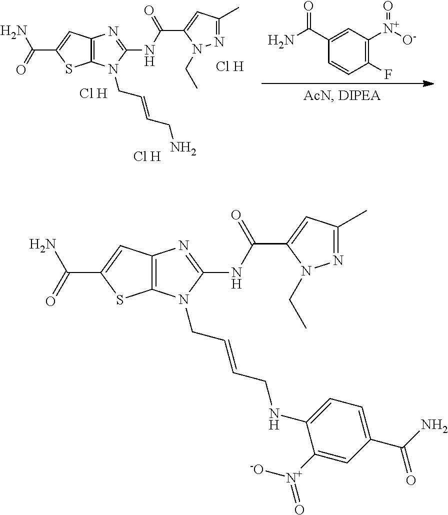

To a stirred suspension of 3-[(E)-4-aminobut-2-enyl]-2-[(2-ethyl-5-methyl-pyrazole-3-carbonyl)amino]thieno[2,3-d]imidazole-5-carboxamide trihydrochloride (147 mg, 0.3 mmol) in ACN (5 mL) and DIPEA (313 μL, 1.8 mmol) was added 4-fluoro-3-nitro-benzamide (66 mg, 0.36 mmol); mixture was stirred at 90° C. for 1 h. The mixture was concentrated in vacuo, diluted with water and ether then filtered to collect the desired product, dried in oven (125 mg, 75%). [MH]⁺=552.2

### (c) 3-[(E)-4-(2-amino-4-carbamoyl-anilino)but-2-enyl]-2-[(2-ethyl-5-methyl-pyrazole-3-carbonyl)amino]thieno[2,3-d]imidazole-5-carboxamide

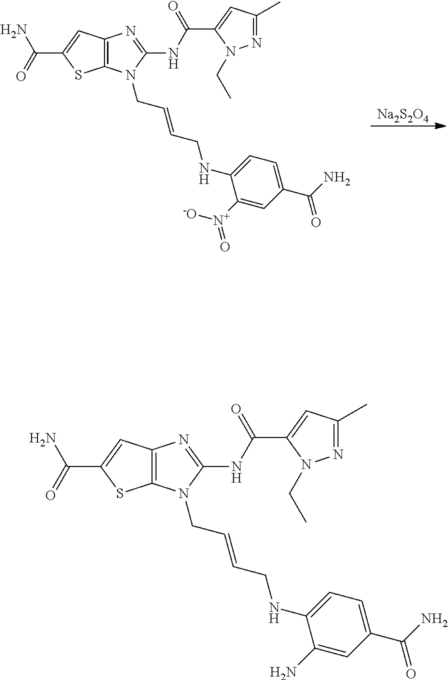

3-[(E)-4-(4-carbamoyl-2-nitro-anilino)but-2-enyl]-2-[(2-ethyl-5-methyl-pyrazole-3-carbonyl)amino]thieno[2,3-d]imidazole-5-carboxamide (125 mg, 0.23 mmol) was stirred in methanol (6 mL) and DIPEA (0.4 mL) then treated with sodium dithionite (237 mg, 1.36 mmol) in water (1 mL). The mixture was stirred at RT for 40 min. The reaction mixture was diluted with water and brine then extracted with ethyl acetate (2×). The organic phase was dried, filtered and concentrated in vacuo then triturated with DCM and diethyl ether giving the desired product as an off-white solid (95 mg, 81%). [MH]+=522.2

### (d) 3-[(E)-4-(2-amino-5-carbamoyl-benzimidazol-1-yl)but-2-enyl]-2-[(2-ethyl-5-methyl-pyrazole-3-carbonyl)amino]thieno[2,3-d]imidazole-5-carboxamide

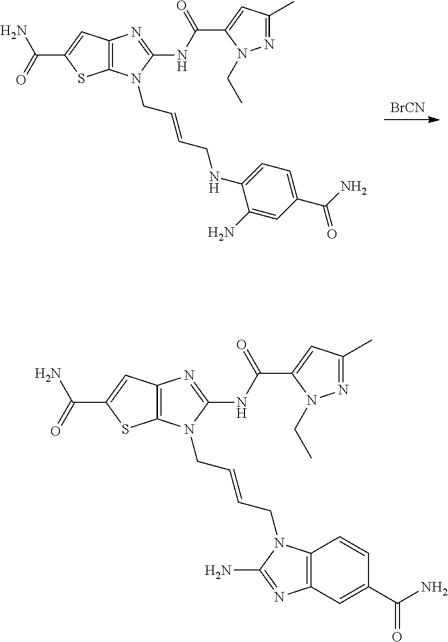 [SMILES: CCn1nc(C)cc1C(=O)Nc1nc2cc(C(N)=O)sc2n1C/C=C/CNc1ccc(C(N)=O)cc1N ; "BrCN" ; CCn1nc(C)cc1C(=O)Nc1nc2cc(C(N)=O)sc2n1C/C=C/Cn1c(N)nc2cc(C(N)=O)ccc21]

To a stirred suspension of 3-[(E)-4-(2-amino-4-carbamoyl-anilino)but-2-enyl]-2-[(2-ethyl-5-methyl-pyrazole-3-carbonyl)amino]thieno[2,3-d]imidazole-5-carboxamide (95 mg, 0.18 mmol) in methanol (4 mL) was added cyanogen bromide (58 mg, 0.55 mmol); reaction was stirred at 60° C. for 3 h. The mixture was concentrated in vacuo and stirred with DCM; DIPEA (0.2 mL) was added. The mixture was sonicated and filtered; filter cake was washed with DCM and diethyl ether to give the desired product (91 mg, 91%) as a pale solid. [MH]⁺=547.3

### (e) 3-[(E)-4-[5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]thieno[2,3-d]imidazole-5-carboxamide Tetrahydrochloride (Compound M)

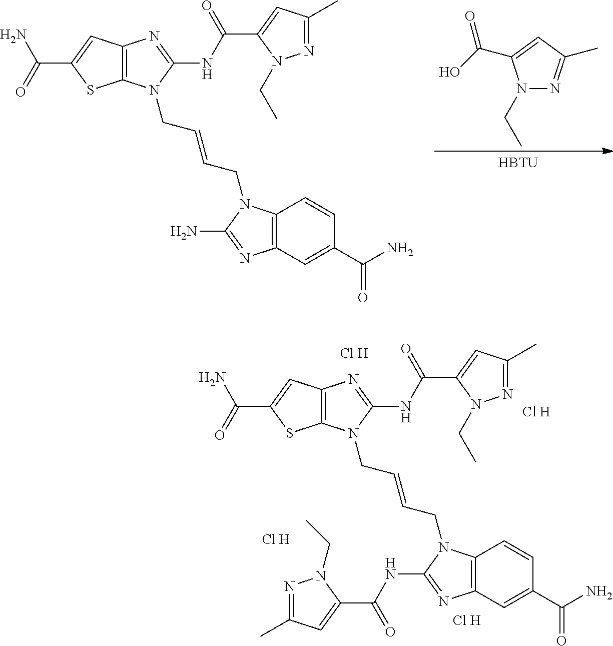

To a stirred solution of 3-[(E)-4-(2-amino-5-carbamoyl-benzimidazol-1-yl)but-2-enyl]-2-[(2-ethyl-5-methyl-pyrazole-3-carbonyl)amino]thieno[2,3-d]imidazole-5-carboxamide (91 mg, 0.17 mmol) and 2-ethyl-5-methyl-pyrazole-3-carboxylic acid (51 mg, 0.33 mmol) in DMF (1.5 mL) was added HBTU (126 mg, 0.33 mmol) followed by DIPEA (87 μL, 0.5 mmol); reaction was stirred at RT for 3 h. The mixture was adsorbed onto silica gel and chromatographed in 0-15% methanol in ethyl acetate. Product containing fractions were concentrated in vacuo and triturated with ethyl acetate and water. The collected solid was stirred in methanol and treated with HCl, 2M in diethyl ether (0.2 mL); the resulting suspension was filtered to collect the desired product (58 mg, 42%). ¹H NMR (d₆-DMSO) δ 8.00 (d, J=4 Hz, 1H), 7.81 (s, 1H), 7.76 (dd, J=8 Hz, 4 Hz, 1H), 7.50 (d, J=8 Hz, 1H), 6.70 (s, 1H), 6.62 (s, 1H), 6.15-6.07 (5.94-5.84 (m, 1H), 4.94 (d, J=8 Hz, 2H), 4.73 (d, J=8 Hz, 2H), 4.58 (q, J=8 Hz, 2H), 4.45 (q, J=8 Hz, 1H), 2.17 (s, 3H), 2.16 (s, 3H), 1.33 (t, J=8 Hz, 3H), 1.26 (t, J=8 Hz, 3H).

### Example 4 Preparation of Compound L

### (a) 4-[[(E)-4-(tert-butoxycarbonylamino)but-2-enyl]amino]-3-nitro-benzoic Acid

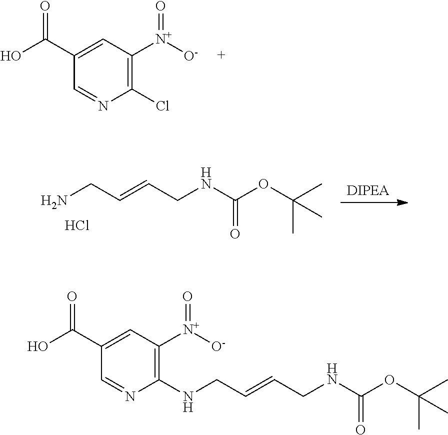

To a stirred solution of 6-chloro-5-nitro-pyridine-3-carboxylic acid (500 mg, 2.46 mmol) in dioxane (5 mL) was added tert-butyl N-[(E)-4-aminobut-2-enyl]carbamate hydrochloride (500 mg, 2.71 mmol) followed by DIPEA (1.09 mL, 6.27 mmol); mixture was stirred at RT overnight. The mixture was diluted with water and acidified to pH ˜3 with 3 N HCl. The resulting suspension was filtered to give the desired product as a yellow solid, dried in oven (636 mg, 73%). ¹H NMR (d₆-DMSO) δ 13.20 (brs, 1H), 9.00 (t, J=6 Hz, 1H), 8.89 (d, J=4 Hz, 1H), 8.74 (d, J=4 Hz, 1H), 6.99-6.91 (m, 1H), 5.72-5.53 (m, 2H), 4.27-4.18 (m, 2H), 3.56-3.47 (m, 2H), 1.36 (s, 9H).

### (b) tert-butyl N-[(E)-4-[(5-carbamoyl-3-nitro-2-pyridyl)amino]but-2-enyl]carbamate

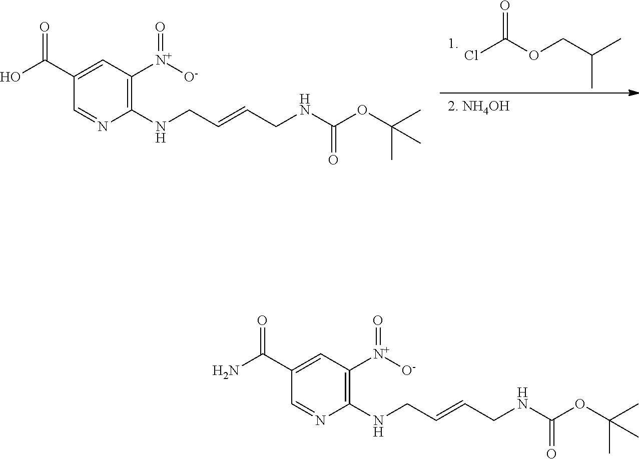

To a stirred solution of 4-[[(E)-4-(tert-butoxycarbonylamino)but-2-enyl]amino]-3-nitro-benzoic acid (630 mg, 1.79 mmol) in THF was added N-methylmorpholine (236 μL, 2.15 mmol). The mixture was cooled to 0° C. and treated with isobutyl chloroformate (256 μL, 1.97 mmol) dropwise. The mixture was warmed to RT, stirred for 20 min then cooled again to 0° C. and treated with ammonium hydroxide (640 μL, 8.96 mmol), dropwise. The mixture was warmed to room temperature and diluted with water. The resulting suspension was filtered to collect the desired product (456 mg, 72%). ¹H NMR (d₆-DMSO) δ 8.92 (d, J=4 Hz, 1H), 8.89 (d, J=4 Hz, 1H), 8.86 (t, J=6 Hz, 1H), 8.10 (brs, 1H), 7.47 (brs, 1H), 6.96 (t, J=6 Hz, 1H), 5.72-5.53 (m, 2H), 4.27-4.16 (m, 2H), 3.58-3.57 (m, 2H), 1.35 (s, 9H).

### (c) 6-[[(E)-4-aminobut-2-enyl]amino]-5-nitro-pyridine-3-carboxamide dihydrochloride

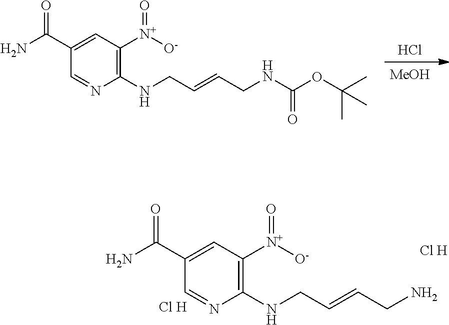

To a stirred suspension of tert-butyl N-[(E)-4-[(5-carbamoyl-3-nitro-2-pyridyl)amino]but-2-enyl]carbamate (351 mg, 1 mmol) in methanol (4 mL) was added HCl, 4 M in dioxane (2.5 mL, 10 mmol). The resulting mixture was stirred for 1 h (mild exotherm observed). The mixture was then concentrated in vacuo giving the desired crude product used directly in the subsequent step (324 mg, quant).

### (d) 6-[[(E)-4-[(5-carbamoyl-3-nitro-2-pyridyl)amino]but-2-enyl]amino]-5-nitro-pyridine-3-carboxamide

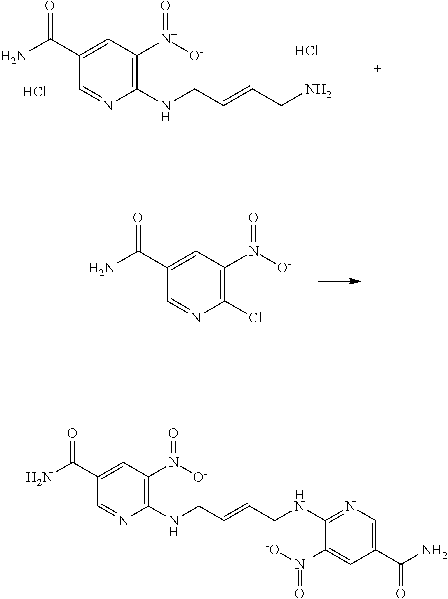

6-[[(E)-4-aminobut-2-enyl]amino]-5-nitro-pyridine-3-carboxamide dihydrochloride (324 mg) and 6-chloro-5-nitro-pyridine-3-carboxamide (210 mg) was stirred in acetonitrile (12 mL) and DIPEA (1 mL) at 70° C. for 4 h. The mixture was concentrated in vacuo then stirred in ethyl acetate overnight. Filtered to collect the crude product (333 mg, 80%). ¹H NMR (d₆-DMSO) δ 8.91 (d, J=4 Hz, 2H), 8.88 (d, J=4 Hz, 2H), 8.88-8.84 (m, 2H), 8.10 (s, 2H), 7.47 (s, 2H), 5.82-5.78 (m, 2H), 4.27-4.21 (m, 4H).

### (e) 2-amino-3-[(E)-4-(2-amino-6-carbamoyl-imidazo[4,5-b]pyridin-3-yl)but-2-enyl]imidazo[4,5-b]pyridine-6-carboxamide Dihydrobromide

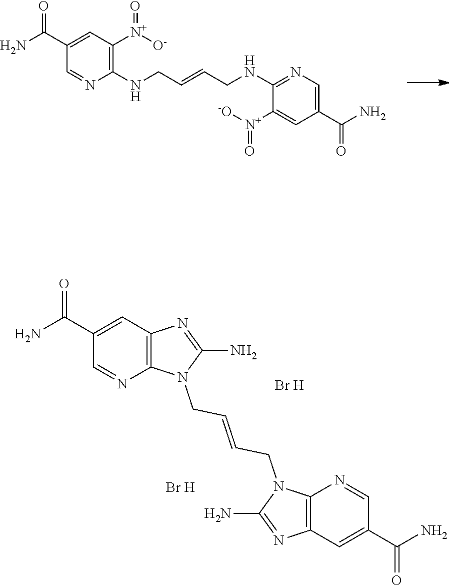

6-[[(E)-4-[(5-carbamoyl-3-nitro-2-pyridyl)amino]but-2-enyl]amino]-5-nitro-pyridine-3-carboxamide (333 mg, 0.8 mmol) was stirred in methanol (10 mL) and ammonium hydroxide (2.9 mL) and treated with sodium dithionite (1.67 g, 9.6 mmol) as a solution I water (10 mL). The mixture was stirred for 4 h, then concentrated in vacuo. The residue was stirred in water and filtered; the filter cake was stirred in methanol, treated with cyanogen bromide (339 mg, 3.2 mmol) and stirred at 60° C. overnight. The mixture was concentrated in vacuo and stirred in diethyl ether; filtered to obtain the desired product (160 mg, 35%).

### (f) 3-[(E)-4-[6-carbamoyl-2-[(2-ethyl-5-methyl-pyrazole-3-carbonyl)amino]imidazo[4,5-b]pyridin-3-yl]but-2-enyl]-2-[(2-ethyl-5-methyl-pyrazole-3-carbonyl)amino]imidazo[4,5-b]pyridine-6-carboxamide (Compound L)

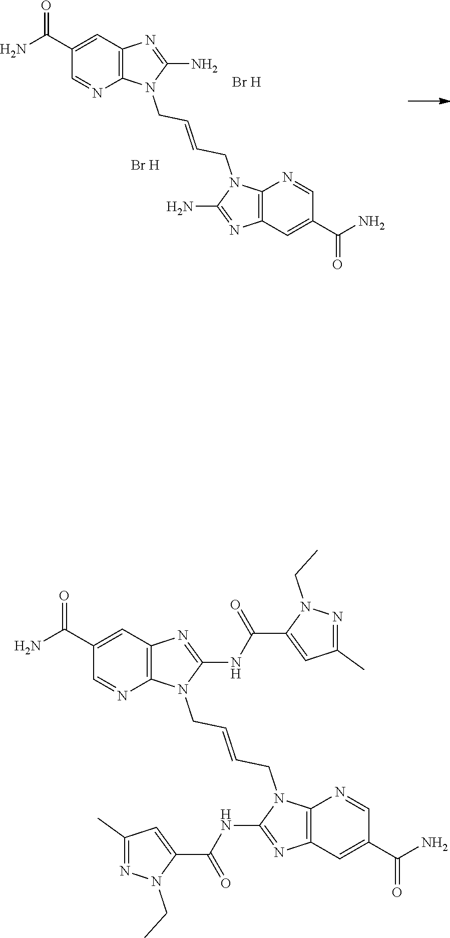

To a stirred solution of 2-ethyl-5-methyl-pyrazole-3-carboxylic acid (142 mg, 0.92 mmol) in DMF (3 mL) was added HBTU (349 mg, 0.92 mmol) followed by DIPEA (258 μL, 1.5 mmol). The mixture was stirred at RT then treated with 2-amino-3-[(E)-4-(2-amino-6-carbamoyl-imidazo[4,5-b]pyridin-3-yl)but-2-enyl]imidazo[4,5-b]pyridine-6-carboxamide dihydrobromide (150 mg, 0.37 mmol); stirred at 80° C. overnight. The mixture was diluted with ethyl acetate and filtered; the filter cake was stirred in methanol and filtered to collect the desired product as a brown solid (70 mg, 39%). [MH]+=679.4

### Example 5 Preparation of Compound F

### (a) benzyl N-[(E)-4-[(5-carbamoyl-3-nitro-2-pyridyl)amino]but-2-enyl]carbamate

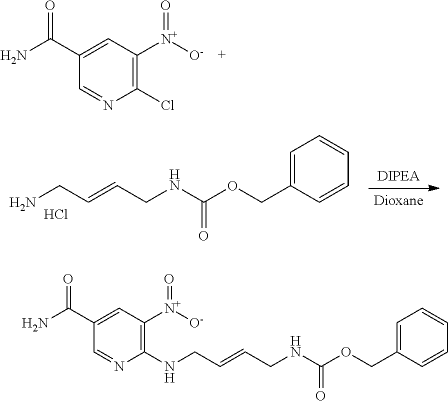

To a stirred suspension of 6-chloro-5-nitro-pyridine-3-carboxamide (400 mg, 2.0 mmol) and benzyl N-[(E)-4-aminobut-2-enyl]carbamate hydrochloride (610 mg, 2.4 mmol) in dioxane was added DIPEA (742 μL, 6.0 mmol); stirred for 1 h. The mixture was diluted with ethyl acetate and water then filtered to collect the desired product as a yellow solid (750 mg, 97%). ¹H NMR (d₆-DMSO) δ 8.92 (d, J=4 Hz, 1H), 8.89 (d, J=4 Hz, 1H), 8.88-8.85 (m, 1H), 8.11 (s, 1H), 7.47 (s, 1H), 7.45-7.39 (m, 1H), 7.39-7.28 (m, 5H), 5.75-5.57 (m, 2H), 5.02 (s, 2H), 4.26-4.20 (m, 2H), 3.65-3.59 (m, 2H).

### (b) benzyl N-[(E)-4-[(3-amino-5-carbamoyl-2-pyridyl)amino]but-2-enyl]carbamate

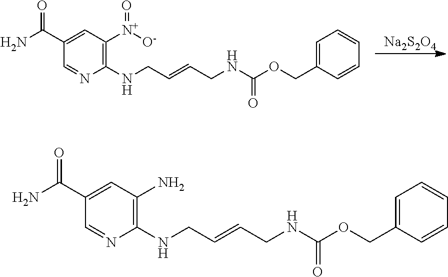

To a stirred suspension of benzyl N-[(E)-4-[(5-carbamoyl-3-nitro-2-pyridyl)amino]but-2-enyl]carbamate (300 mg, 0.78 mmol) in methanol (6 mL) and ammonium hydroxide (1.35 mL, 0.02 mmol) was added sodium dithionite as a solution in water (3 mL); stirred at room temperature for 2 h. The mixture was diluted with water and extracted with ethyl acetate (3×). The combined organics were washed with brine, dried, filtered and concentrated in vacuo; the residue was used directly in the subsequent reaction. [MH]=356.2

### (c) benzyl N-[(E)-4-(2-amino-6-carbamoyl-imidazo[4,5-b]pyridin-3-yl)but-2-enyl]carbamate

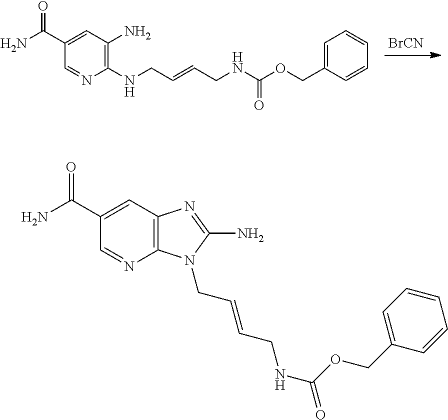

To a stirred solution of benzyl N-[(E)-4-[(3-amino-5-carbamoyl-2-pyridyl)amino]but-2-enyl]carbamate (276 mg, 0.78 mmol) in methanol was added cyanogen bromide (83 mg, 0.78 mmol); stirred at 60° C. overnight. More cyanogen bromide (166 mg) was added, stirred overnight at 60° C. The mixture was treated with 2 M NH₃ in methanol, adsorbed onto silica gel and chromatographed in 0-10% (2M NH₃ in MeOH) in DCM. The product containing fractions were concentrated in vacuo giving a brown oil (60 mg, 20%). [MH]+=381.2

### (d) benzyl N-[(E)-4-[6-carbamoyl-2-[(2-ethyl-5-methyl-pyrazole-3-carbonyl)amino]imidazo[4,5-b]pyridin-3-yl]but-2-enyl]carbamate (Compound F)

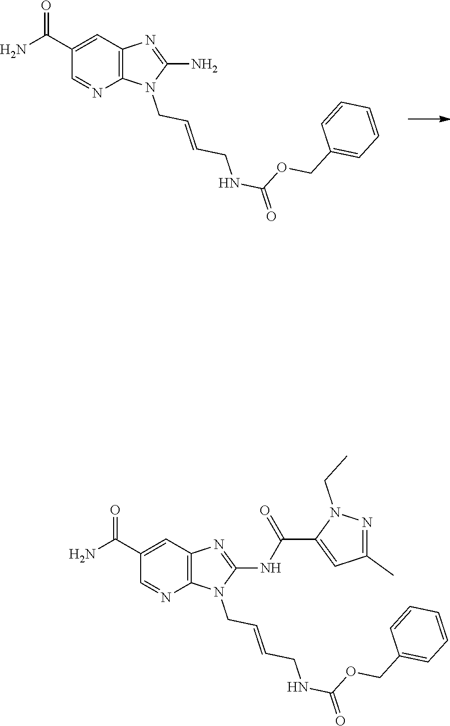 [SMILES: NC(=O)c1cnc2c(c1)nc(N)n2C/C=C/CNC(=O)OCc1ccccc1 ; CCn1nc(C)cc1C(=O)Nc1nc2cc(C(N)=O)cnc2n1C/C=C/CNC(=O)OCc1ccccc1]

To a stirred solution of 2-ethyl-5-methyl-pyrazole-3-carboxylic acid (36 mg, 0.24 mmol) in DMF (2 mL) was added HBTU (91 mg, 0.24 mmol) followed by DIPEA. The mixture was stirred at RT for 15 min then treated with benzyl N-[(E)-4-(2-amino-6-carbamoyl-imidazo[4,5-b]pyridin-3-yl)but-2-enyl]carbamate (60 mg, 0.16 mmol); stirred overnight. The mixture was diluted with water and saturated sodium bicarbonate then extracted with ethyl acetate (4×). The combined organics were washed with brine, dried, filtered and concentrated in vacuo then chromatographed in 0-10% (2M NH₃ in MeOH) in DCM. The product containing fractions were concentrated in vacuo then triturated with hexanes giving the desired product as a beige solid (25 mg, 31%). ¹H NMR (d₆-DMSO) δ 12.89 (s, 1H), 8.76 (s, 1H), 8.15 (s, 1H), 7.53 (s, 1H), 7.40 (s, 1H), 7.36-7.18 (m, 5H), 6.71 (s, 1H), 5.84-5.73 (m, 1H), 5.73-5.61 (m, 1H), 4.97 (s, 2H), 4.85-4.75 (m, 2H), 4.67-4.56 (m, 2H), 3.65-3.54 (m, 2H), 2.19 (s, 3H), 1.36 (t, J=8 Hz, 3H).

### Example 6 Preparation of Compound D

### (a) tert-butyl N-[(E)-4-[(3-amino-5-carbamoyl-2-pyridyl)amino]but-2-enyl]carbamate

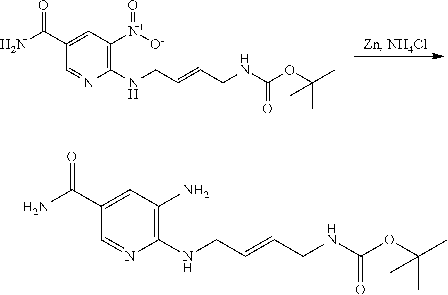

To a stirred solution of tert-butyl N-[(E)-4-[(5-carbamoyl-3-nitro-2-pyridyl)amino]but-2-enyl]carbamate (2.48 g, 7.06 mmol) in methanol (30 mL), THF (30 mL) and water (30 mL) was added ammonium chloride (3.78 g, 70.6 mmol) and zinc dust (4.61 g, 70.6 mmol); stirred at RT for 15 min. The mixture was filtered to remove zinc salts and concentrated in vacuo to remove organic solvents. The concentrate was diluted with ethyl acetate and washed with saturated sodium bicarbonate (1×) and brine (1×). The organic phase was dried, filtered and concentrated in vacuo giving the desired crude product as an orange/red foam (2.2 g, 97%). [MH]⁺=322.2

### (b) tert-butyl N-[(E)-4-(2-amino-6-carbamoyl-imidazo[4,5-b]pyridin-3-yl)but-2-enyl]carbamate

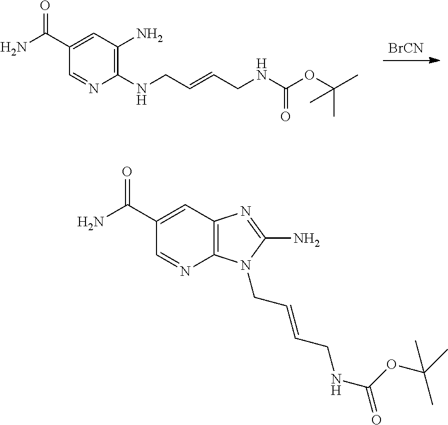

To a stirred solution of tert-butyl N-[(E)-4-[(3-amino-5-carbamoyl-2-pyridyl)amino]but-2-enyl]carbamate (2.2 g, 6.8 mmol) was added cyanogen bromide (1.5 g, 14.1 mmol); stirred at 60° C. overnight. The mixture was then concentrated in vacuo, diluted with water, saturated sodium bicarbonate and brine then extracted with ethyl acetate (5×). The combined organics were dried, filtered and concentrated in vacuo then triturated with ethyl acetate and diethyl ether; filtered to give the desired product as a pink solid (1.29 g, 54%). [MH]⁺=347.2

### (c) tert-butyl N-[(E)-4-[6-carbamoyl-2-[(2-ethyl-5-methyl-pyrazole-3-carbonyl)amino]imidazo[4,5-b]pyridin-3-yl]but-2-enyl]carbamate (Compound D)

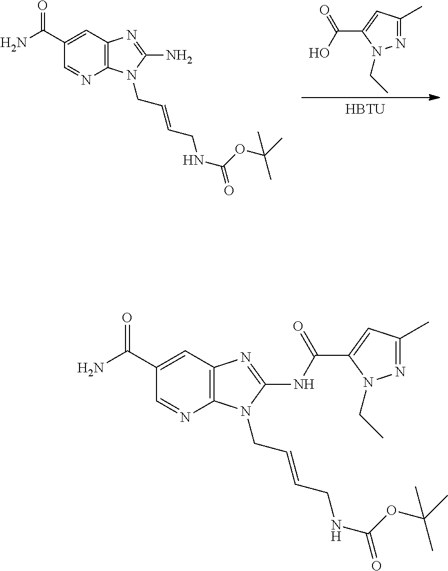 [SMILES: CC(C)(C)OC(=O)NC/C=C/Cn1c(N)nc2cc(C(N)=O)cnc21 ; "HBTU" ; CCn1nc(C)cc1C(=O)O ; CCn1nc(C)cc1C(=O)Nc1nc2cc(C(N)=O)cnc2n1C/C=C/CNC(=O)OC(C)(C)C]

To a stirred solution of tert-butyl N-[(E)-4-(2-amino-6-carbamoyl-imidazo[4,5-b]pyridin-3-yl)but-2-enyl]carbamate (693 mg, 2.0 mmol) and 2-ethyl-5-methyl-pyrazole-3-carboxylic acid (401 mg, 2.6 mmol) in DMF (5 mL) was added HBTU (1.14 g, 3.0 mmol) followed by DIPEA (1.05 mL, 6.0 mmol). The resulting mixture was stirred at RT overnight. Quenched with ice and sodium carbonate, extracted with ethyl acetate (3×). The combined organics were washed with brine (2×), dried, filtered and concentrated in vacuo. The residue was stirred in ethyl acetate, water and diethyl ether then filtered; filter cake was dried in oven to give the desired product (645 mg, 67%). ¹H NMR (d₆-DMSO) δ 8.65 (s, 1H), 8.50 (s, 1H), 7.94 (s, 1H), 7.31 (s, 1H), 6.92 (m, 1H), 6.56 (s, 1H), 5.82-5.69 (m, 1H), 5.60-5.47 (m, 1H), 4.88-4.78 (m, 2H), 4.28-4.14 (m, 2H), 3.53-3.41 (m, 2H), 2.10 (s, 3H), 1.33 (s, 9H), 0.92-0.79 (m, 3H).

### Example 7 Preparation of Compound E

### tert-butyl N-[4-[6-carbamoyl-2-[(2-ethyl-5-methyl-pyrazole-3-carbonyl)amino]imidazo[4,5-b]pyridin-3-yl]butyl]carbamate (Compound E)

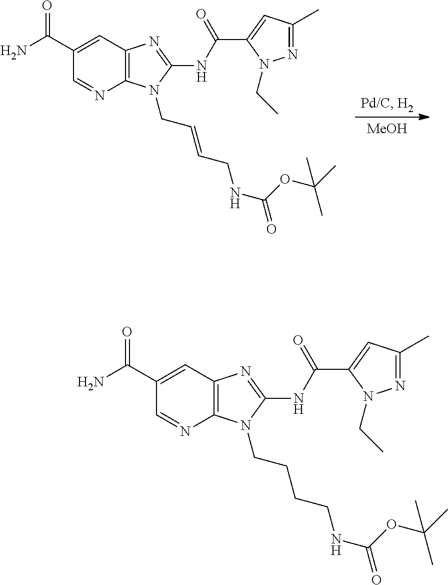 [SMILES: "MeOH" ; CCn1nc(C)cc1C(=O)Nc1nc2cc(C(N)=O)cnc2n1C/C=C/CNC(=O)OC(C)(C)C ; CCn1nc(C)cc1C(=O)Nc1nc2cc(C(N)=O)cnc2n1CCCCNC(=O)OC(C)(C)C]

tert-butyl N-[(E)-4-[6-carbamoyl-2-[(2-ethyl-5-methyl-pyrazole-3-carbonyl)amino]imidazo[4,5-b]pyridin-3-yl]but-2-enyl]carbamate (Compound D, 100 mg, 0.207 mmol) was stirred in methanol (20 mL) and treated with palladium, 10 wt. % on activated carbon (100 mg). The suspension was stirred under an atmosphere of hydrogen (balloon pressure) overnight. The mixture was acidified with 1 N HCl to aid dissolution of product, then filtered to remove palladium/carbon. The filtrate was neutralized with sodium bicarbonate, concentrated to remove methanol, diluted with water and filtered. The filter cake was stirred in methanol, concentrated in vacuo then triturated with diethyl ether to give the desired product (39 mg, 39%). ¹H NMR (d₆-DMSO) δ 8.65 (s, 1H), 8.47 (s, 1H), 7.93 (s, 1H), 7.30 (s, 1H), 6.84-6.74 (m, 1H), 6.56 (s, 1H), 4.33-4.07 (m, 4H), 3.00-2.87 (m, 2H), 2.08 (s, 3H), 1.86-1.69 (m, 2H), 1.34 (s, 9H), 0.90-0.74 (m, 3H).

### Example 8 Preparation of Compound A

### (a) 1-[(E)-4-aminobut-2-enyl]-2-[(2-ethyl-5-methyl-pyrazole-3-carbonyl)amino]-7-methoxy-benzimidazole-5-carboxamide dihydrochloride

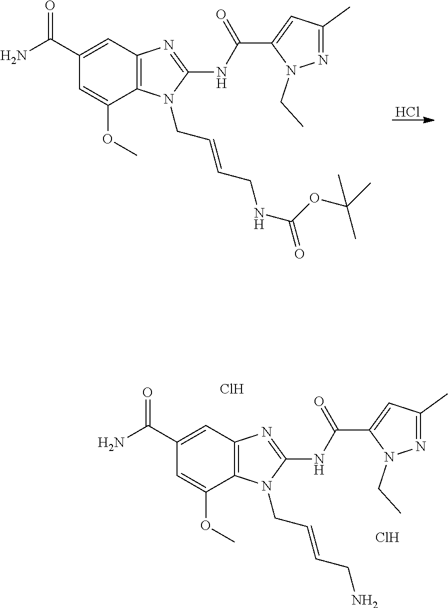 [SMILES: CCn1nc(C)cc1C(=O)Nc1nc2cc(C(N)=O)cc(OC)c2n1C/C=C/CNC(=O)OC(C)(C)C ; Cl ; Cl ; CCn1nc(C)cc1C(=O)Nc1nc2cc(C(N)=O)cc(OC)c2n1C/C=C/CN ; Cl]

tert-butyl N-[(E)-4-[5-carbamoyl-2-[(2-ethyl-5-methyl-pyrazole-3-carbonyl)amino]-7-methoxy-benzimidazol-1-yl]but-2-enyl]carbamate (116 mg, 0.23 mmol) was stirred in methanol and treated with hydrogen chloride, 4 M in dioxane (8 mL). The resulting mixture was stirred for 1 h, then concentrated in vacuo giving the desired product as a white solid (109 mg, quant.). [MH]=412.3

### (b) 1-[(E)-4-[(5-carbamoyl-3-nitro-2-pyridyl)amino]but-2-enyl]-2-[(2-ethyl-5-methyl-pyrazole-3-carbonyl)amino]-7-methoxy-benzimidazole-5-carboxamide

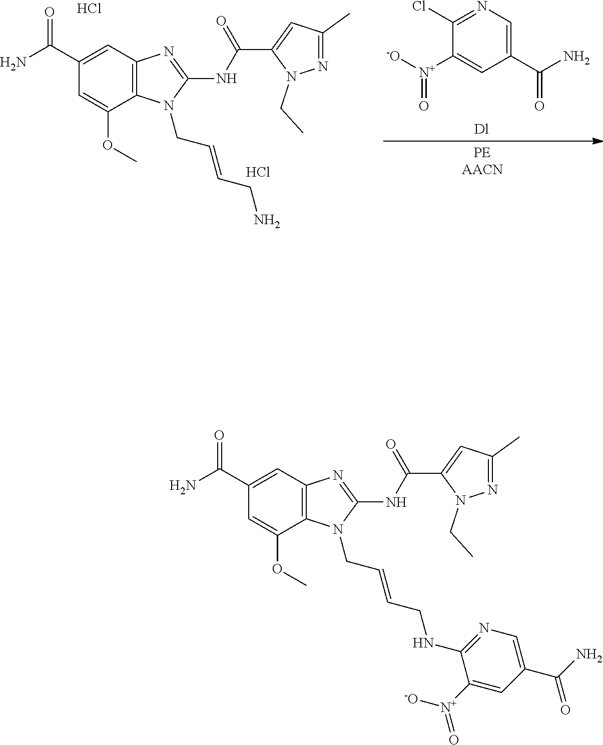

1-[(E)-4-aminobut-2-enyl]-2-[(2-ethyl-5-methyl-pyrazole-3-carbonyl)amino]-7-methoxy-benzimidazole-5-carboxamide dihydrochloride (109 mg, 0.23 mmol) and 6-chloro-5-nitro-pyridine-3-carboxamide (55 mg, 0.27 mmol) were stirred in ACN (5 mL) and treated with DIPEA (394 μL, 292 mg); stirred at 90° C. for 5 h. The mixture was concentrated in vacuo, triturated with diethyl ether and water then filtered to collect the desired crude product (131 mg). [MH]⁺=577.3

### (c) 1-[(E)-4-[(3-amino-5-carbamoyl-2-pyridyl)amino]but-2-enyl]-2-[(2-ethyl-5-methyl-pyrazole-3-carbonyl)amino]-7-methoxy-benzimidazole-5-carboxamide

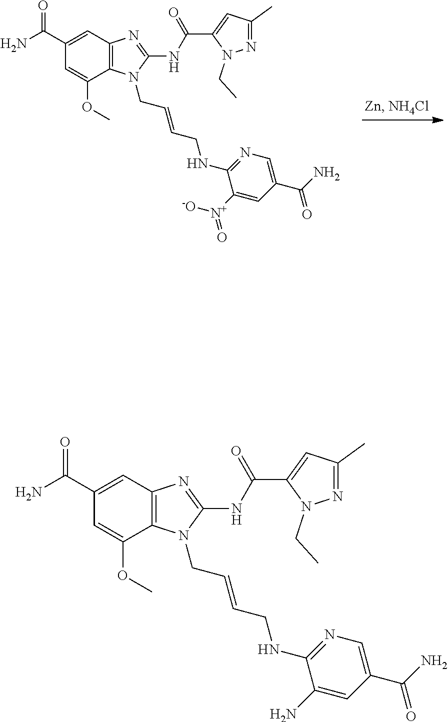

To a stirred suspension of 1-[(E)-4-[(5-carbamoyl-3-nitro-2-pyridyl)amino]but-2-enyl]-2-[(2-ethyl-5-methyl-pyrazole-3-carbonyl)amino]-7-methoxy-benzimidazole-5-carboxamide (131 mg) in methanol (6 mL), THF (6 mL) and water (6 mL) was added ammonium chloride (280 mg) and zinc dust (280 mg); reaction was stirred for 20 min. The mixture was filtered, concentrated to remove organic solvents and basified with potassium carbonate and brine; extracted with ethyl acetate (2×). The combined organics were dried, filtered and concentrated giving the desired crude product (56 mg). [MH]⁺=547.3

### (d) 2-amino-3-[(E)-4-[5-carbamoyl-2-[(2-ethyl-5-methyl-pyrazole-3-carbonyl)amino]-7-methoxy-benzimidazol-1-yl]but-2-enyl]imidazo[4,5-b]pyridine-6-carboxamide Hydrobromide

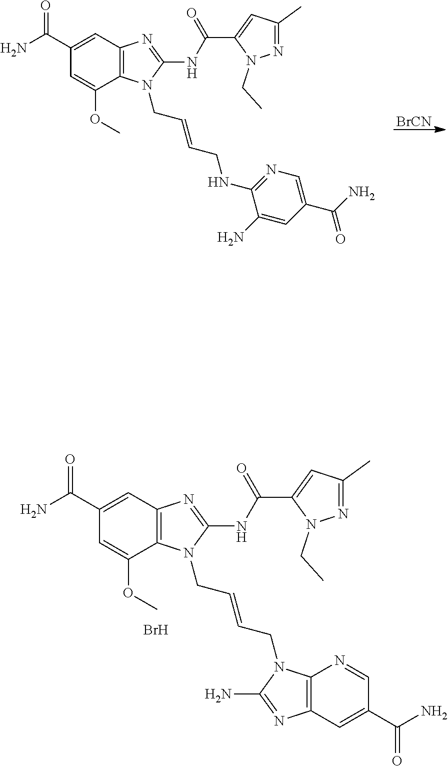 [SMILES: CCn1nc(C)cc1C(=O)Nc1nc2cc(C(N)=O)cc(OC)c2n1C/C=C/CNc1ncc(C(N)=O)cc1N ; "BrCN" ; CCn1nc(C)cc1C(=O)Nc1nc2cc(C(N)=O)cc(OC)c2n1C/C=C/Cn1c(N)nc2cc(C(N)=O)cnc21 ; Br]

1-[(E)-4-[(3-amino-5-carbamoyl-2-pyridyl)amino]but-2-enyl]-2-[(2-ethyl-5-methyl-pyrazole-3-carbonyl)amino]-7-m ethoxy-benzimidazole-5-carboxamide (56 mg, 0.10 mmol) was stirred in methanol (3 mL) and treated with cyanogen bromide (56 mg, 0.53 mmol); stirred at 60° C. overnight. The mixture was concentrated in vacuo then triturated with diethyl ether to give the desired product (45 mg, 67%). [MH]⁺=572.2

### (e) 3-[(E)-4-[5-carbamoyl-2-[(2-ethyl-5-methyl-pyrazole-3-carbonyl)amino]-7-methoxy-benzimidazol-1-yl]but-2-enyl]-2-[(2-ethyl-5-methyl-pyrazole-3-carbonyl)amino]imidazo[4,5-b]pyridine-6-carboxamide (Compound A)

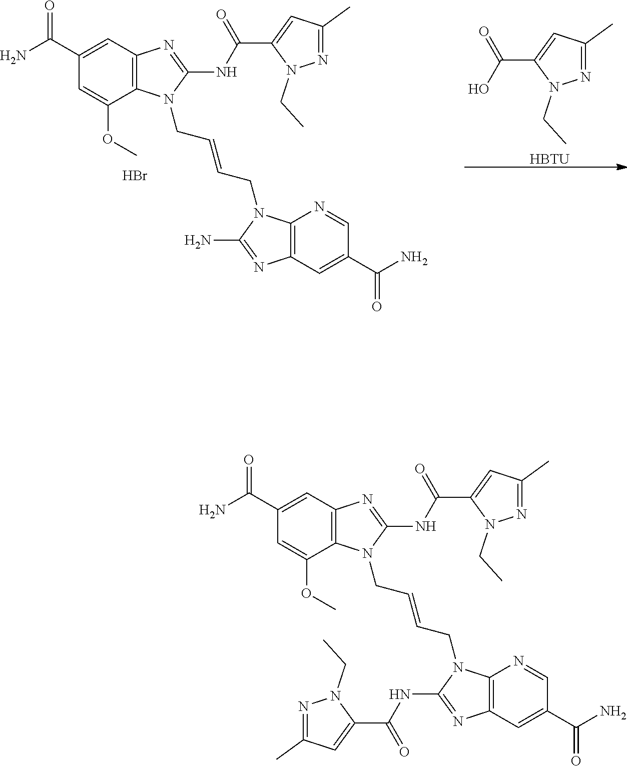 [SMILES: CCn1nc(C)cc1C(=O)Nc1nc2cc(C(N)=O)cc(OC)c2n1C/C=C/Cn1c(N)nc2cc(C(N)=O)cnc21 ; Br ; CCn1nc(C)cc1C(=O)O ; "HBTU" ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OC)c21]

To a stirred solution of 2-amino-3-[(E)-4-[5-carbamoyl-2-[(2-ethyl-5-methyl-pyrazole-3-carbonyl)amino]-7-methoxy-benzimidazol-1-yl]but-2-enyl]imidazo[4,5-b]pyridine-6-carboxamide hydrobromide (45 mg, 0.079 mmol) and 2-ethyl-5-methyl-pyrazole-3-carboxylic acid (15 mg, 0.094 mmol) in DMF (2 mL) was added HBTU (45 mg, 0.12 mmol) followed by DIPEA (41 μL, 0.24 mmol); stirred overnight. The mixture was diluted with diethyl ether and filtered; the filter cake was stirred in methanol then filtered to collect the desired product as a beige solid (8 mg, 16%). [MH]+=354.7 (doubly protonated)

### Example 9

### (a) 1-[(E)-4-[(5-carbamoyl-3-nitro-2-pyridyl)amino]but-2-enyl]-2-[(2-ethyl-5-methyl-pyrazole-3-carbonyl)amino]-7-methyl-benzimidazole-5-carboxamide

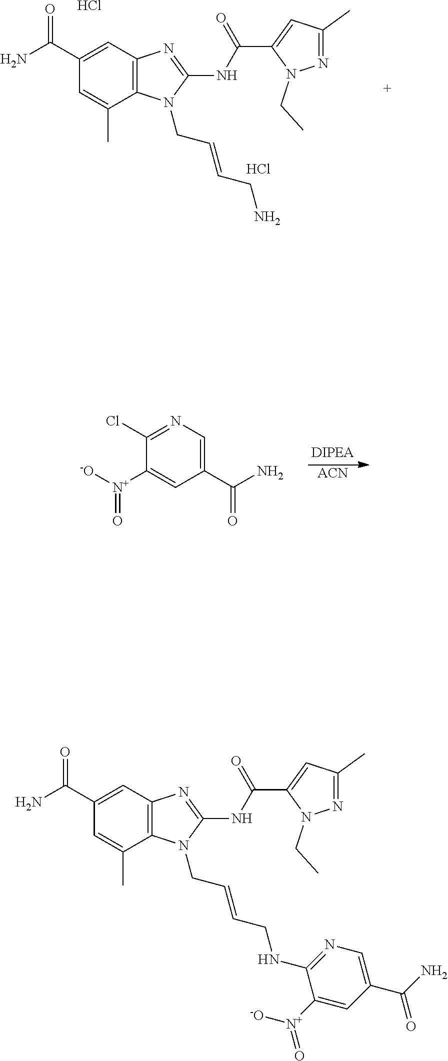

1-[(E)-4-aminobut-2-enyl]-2-[(2-ethyl-5-methyl-pyrazole-3-carbonyl)amino]-7-methyl-benzimidazole-5-carboxamide dihydrochloride (398 mg, 0.85 mmol) and 6-chloro-5-nitro-pyridine-3-carboxamide (172 mg, 0.85 mmol) was stirred in ACN (20 mL) and treated with DIPEA (1.5 mL, 8.55 mmol); stirred at 90° C. for 1 h. The mixture was concentrated in vacuo then diluted with ether. The suspension was filtered and the filter cake was washed with water and ether. Filter cake was dried in oven to obtain desired crude product (476 mg, quant); used directly in the subsequent reaction. [MH]+⁼561.3

### (b) 1-[(E)-4[(3-amino-5-carbamoyl-2-pyridyl)amino]but-2-enyl]-2-[(2-ethyl-5-methyl-pyrazole-3-carbonyl)amino]-7-methyl-benzimidazole-5-carboxamide

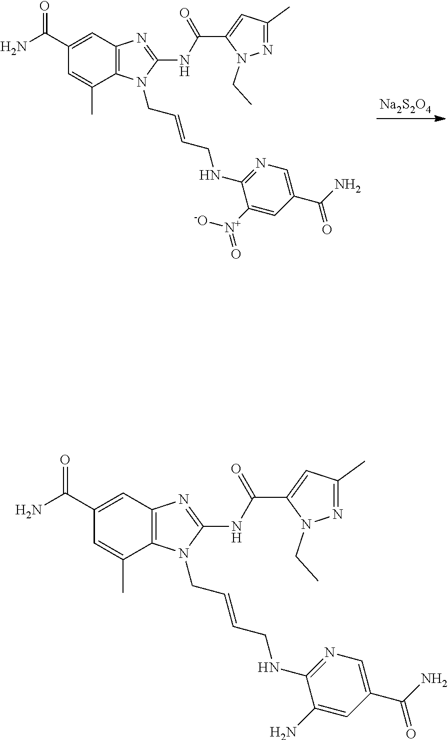

1-[(E)-4-[(5-carbamoyl-3-nitro-2-pyridyl)amino]but-2-enyl]-2-[(2-ethyl-5-methyl-pyrazole-3-carbonyl)amino]-7-methyl-benzimidazole-5-carboxamide (476 mg, 0.85 mmol) was stirred in methanol (15 mL), water (15 mL) and THF (15 mL) then treated with ammonium chloride (450 mg) and zinc dust (550 mg); stirred for 30 min. The mixture was concentrated in vacuo, basified with ammonium hydroxide and filtered. The filter cake was washed with water and diethyl ether; dried in oven to give the desired product (420 mg, 92%). [MH]⁺=531.3

### (c) 2-amino-3-[(E)-4-[5-carbamoyl-2-[(2-ethyl-5-methyl-pyrazole-3-carbonyl)amino]-7-methyl-benzimidazol-1-yl]but-2-enyl]imidazo[4,5-b]pyridine-6-carboxamide

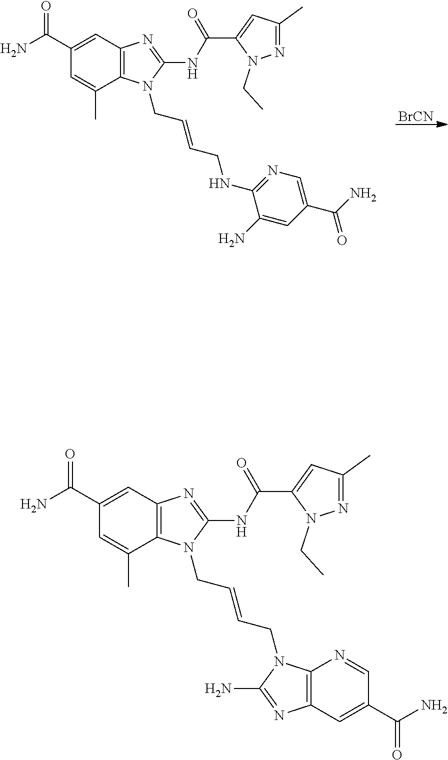 [SMILES: CCn1nc(C)cc1C(=O)Nc1nc2cc(C(N)=O)cc(C)c2n1C/C=C/CNc1ncc(C(N)=O)cc1N ; "BrCN" ; CCn1nc(C)cc1C(=O)Nc1nc2cc(C(N)=O)cc(C)c2n1C/C=C/Cn1c(N)nc2cc(C(N)=O)cnc21]

1-[(E)-4-[(3-amino-5-carbamoyl-2-pyridyl)amino]but-2-enyl]-2-[(2-ethyl-5-methyl-pyrazole-3-carbonyl)amino]-7-methyl-benzimidazole-5-carboxamide (280 mg, 0.53 mmol) was stirred in methanol (5 mL) and treated with cyanogen bromide (167 mg, 1.6 mmol); stirred at 60° C. overnight. The mixture was concentrated in vacuo; resuspended in methanol and treated with trimethylamine (2 mL). Concentrated in vacuo then triturated with DCM; filtered to collect desired product (200 mg, 68%). [MH]=556.3

### Example 10 Preparation of Compound B

### (a) 3-[(E)-4-[5-carbamoyl-2-[(2-ethyl-5-methyl-pyrazole-3-carbonyl)amino]-7-methyl-benzimidazol-1-yl]but-2-enyl]-2-[(2-ethyl-5-methyl-pyrazole-3-carbonyl)amino]imidazo[4,5-b]pyridine-6-carboxamide Tetrahydrochloride (Compound B)

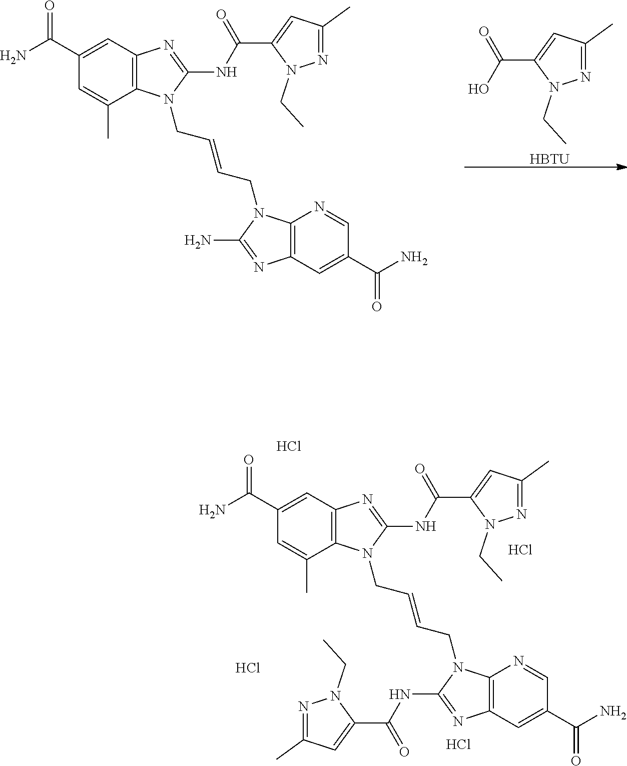

To a stirred solution of 2-amino-3-[(E)-4-[5-carbamoyl-2-[(2-ethyl-5-methyl-pyrazole-3-carbonyl)amino]-7-methyl-benzimidazol-1-yl]but-2-enyl]imidazo[4,5-b]pyridine-6-carboxamide (100 mg, 0.18 mmol) and 2-ethyl-5-methyl-pyrazole-3-carboxylic acid (56 mg, 0.36 mmol) in DMF (1.5 mL) was added HBTU (137 mg, 0.36 mmol) followed DIPEA (94 μL, 0.54 mmol); stirred overnight at RT. The mixture was diluted with ethyl acetate and filtered. The filter cake was stirred in ethyl acetate and water then filtered. The filter cake was stirred in methanol and treated with 4 M HCl in dioxane to dissolve, filtered to remove insoluble material. The filtrate was concentrated in vacuo and triturated with acetone to give the desired product as a hydrochloride salt (45 mg, 30%). ¹H NMR (d₆-DMSO) δ 8.72 (s, 1H), 8.19-8.11 (m, 2H), 7.92-7.83 (m, 2H), 7.56-7.47 (m, 2H), 7.29 (s, 1H), 6.56 (s, 1H), 6.53 (s, 1H), 5.95-5.86 (m, 1H), 5.63-5.51 (m, 1H), 5.02-4.94 (m, 1H), 4.82-4.74 (m, 2H), 4.58-4.48 (m, 4H), 2.54 (s, 3H), 2.16-2.07 (m, 6H), 1.33-1.22 (m, 6H).

### Example 11 Preparation of Compound C

### (a) N-[6-carbamoyl-3-[(E)-4-[5-carbamoyl-2-[(2-ethyl-5-methyl-pyrazole-3-carbonyl)amino]-7-methyl-benzimidazol-1-yl]but-2-enyl]imidazo[4,5-b]pyridin-2-yl]-2-methyl-thiazole-5-carboxamide Hydrochloride (Compound C)

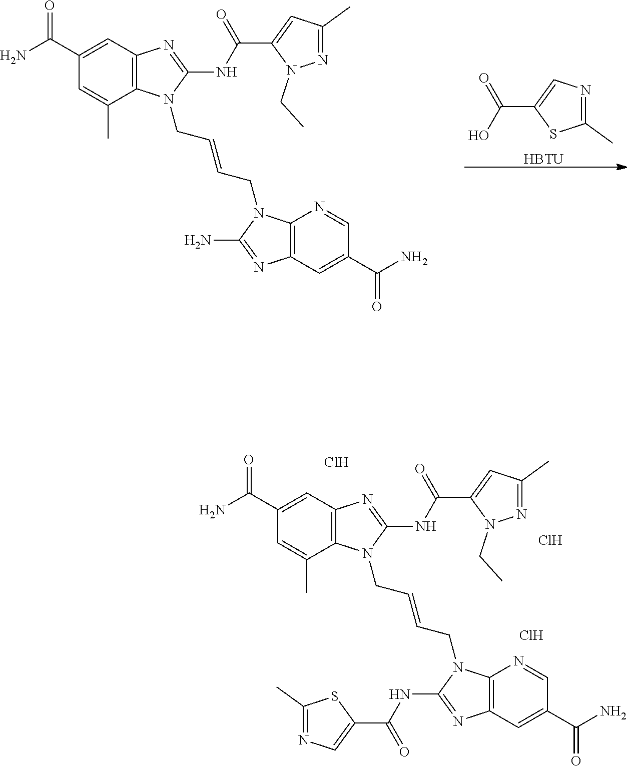

To a stirred solution of 2-amino-3-[(E)-4-[5-carbamoyl-2-[(2-ethyl-5-methyl-pyrazole-3-carbonyl)amino]-7-methyl-benzimidazol-1-yl]but-2-enyl]imidazo[4,5-b]pyridine-6-carboxamide (100 mg, 0.18 mmol) and 2-methylthiazole-5-carboxylic acid (52 mg, 0.36 mmol) in DMF (1.5 mL) was added HBTU (137 mg, 0.36 mmol) followed DIPEA (94 μL, 0.54 mmol); stirred overnight at RT. The mixture was diluted with ethyl acetate and filtered. The filter cake was stirred in ethyl acetate and water then filtered. The filter cake was stirred in methanol and treated with 4 M HCl in dioxane to dissolve, filtered to remove insoluble material. The filtrate was concentrated in vacuo and triturated with acetone to give the desired product as a hydrochloride salt (70 mg, 46%). ¹H NMR (d₆-DMSO) δ 8.72 (s, 1H), 8.18-8.10 (m, 3H), 7.91-7.82 (m, 2H), 7.54 (s, 1H), 7.48 (s, 1H), 7.28 (s, 1H), 6.53 (s, 1H), 6.01-5.92 (m, 1H), 5.65-5.52 (m, 1H), 5.03-4.96 (m, 2H), 4.81-4.72 (m, 2H), 4.58-4.57 (m, 2H), 2.63 (s, 3H), 2.54 (s, 3H), 2.12 (s, 3H), 1.28 (t, J=8 Hz, 3H).

### Example 12 Preparation of Compound N

### (a) 4-chloro-3-hydroxy-5-nitro-benzamide

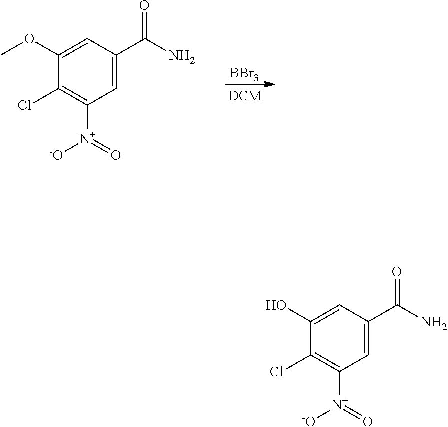

To as stirred suspension of 4-chloro-3-methoxy-5-nitro-benzamide (1.85 g, 8.02 mmol) in DCM (20 mL) was added boron tribromide (3.04 mL, 32.08 mmol), slowly; stirred overnight at room temperature. The mixture was poured over ice and stirred in water giving a suspension which was filtered. The filter cake was stirred in water (acidified with minimal HCl); filtered then dried in oven to give the desired product as a white solid (1.33 g, 76%). ¹H NMR (d₆-DMSO) δ 11.53 (brs, 1H), 8.18 (brs, 1H), 7.93 (d, J=4 Hz, 1H), 7.72 (d, J=4 Hz, 1H), 7.67 (brs, 1H).

### (b) 4-chloro-3-[2-(difluoromethoxy)ethoxy]-5-nitro-benzamide

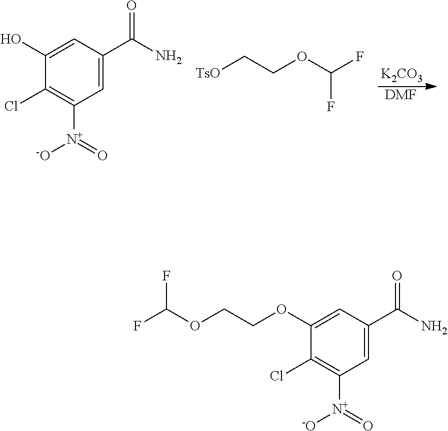

To a stirred suspension of 4-chloro-3-hydroxy-5-nitro-benzamide (1.4 g, 6.46 mmol) and potassium carbonate (1.78 g, 12.9 mmol) in DMF (10 mL) was added 2-(difluoromethoxy)ethyl 4-methylbenzenesulfonate (1.72 g, 6.46 mmol); stirred at 65° C. for 2 h. The mixture was diluted with ethyl acetate then washed with brine (1×), brine:water (1×) and brine (1×). The organic phase was dried, filtered and concentrated in vacuo then chromatographed in 0-70% ethyl acetate in hexanes giving the desired product as a white solid (1.2 g, 60%). ¹H NMR (d₆-DMSO) δ 8.28 (brs, 1H), 8.08 (d, J=4 Hz, 1H), 7.91 (d, J=4 Hz, 1H), 7.80 (brs, 1H), 6.78 (t, J=76 Hz, 1H), 4.51-4.44 (m, 2H), 4.29-4.24 (m, 2H).

### (c) 3-[(E)-4-aminobut-2-enyl]-2-[(2-ethyl-5-methyl-pyrazole-3-carbonyl)amino]thieno[2,3-d]imidazole-5-carboxamide Trihydrochloride

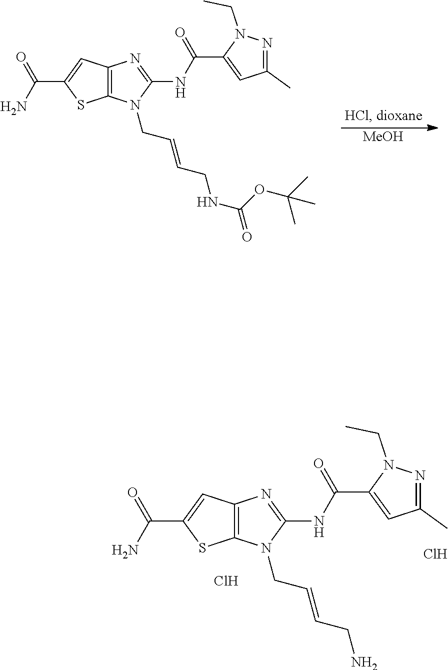 [SMILES: "MeOH" ; CCn1nc(C)cc1C(=O)Nc1nc2cc(C(N)=O)sc2n1C/C=C/CNC(=O)OC(C)(C)C ; CCn1nc(C)cc1C(=O)Nc1nc2cc(C(N)=O)sc2n1C/C=C/CN ; Cl ; Cl]

To a stirred suspension of tert-butyl N-[(E)-4-[5-carbamoyl-2-[(2-ethyl-5-methyl-pyrazole-3-carbonyl)amino]thieno[2,3-d]imidazol-3-yl]but-2-enyl]carbamate (1.56 g, 3.19 mmol) in methanol (8 mL) was added HCl, 4 M in dioxane (8 mL, 31.9 mmol); stirred at room temperature for 3 h. The mixture was concentrated in vacuo to remove excess solvent then stirred in diethyl ether; the resulting suspension was filtered to obtain the desired product (1.54 g, 97%). LCMS: 388.1 (MH⁺).

### (d) 3-[(E)-4-[4-carbamoyl-2-[2-(difluoromethoxy)ethoxy]-6-nitro-anilino]but-2-enyl]-2-[(2-ethyl-5-methyl-pyrazole-3-carbonyl)amino]thieno[2,3-d]imidazole-5-carboxamide

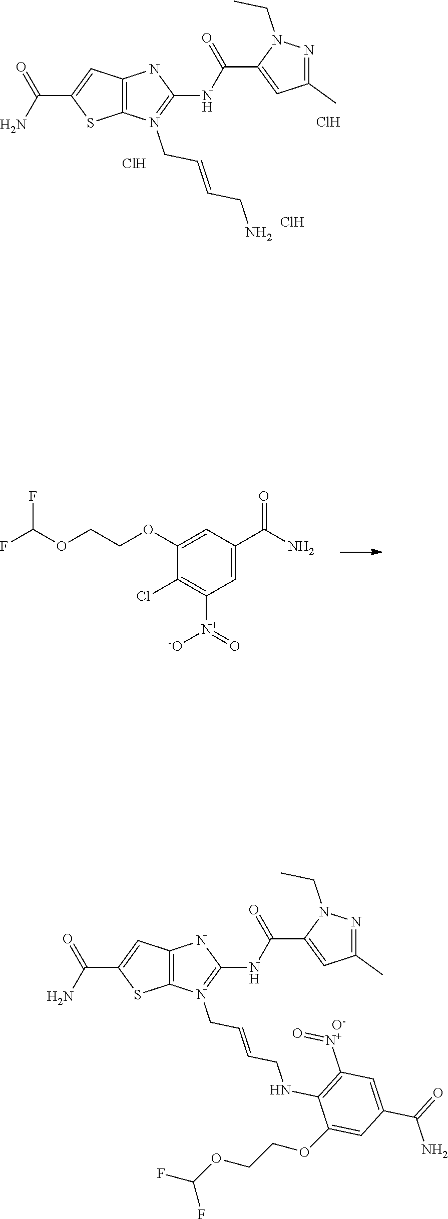

3-[(E)-4-aminobut-2-enyl]-2-[(2-ethyl-5-methyl-pyrazole-3-carbonyl)amino]thieno[2,3-d]imidazole-5-carboxamide trihydrochloride (533 mg, 1.1 mmol) and 4-chloro-3-[2-(difluoromethoxy)ethoxy]-5-nitro-benzamide (500 mg, 1.6 mmol) were stirred in n-propanol (10 mL) then treated with DIPEA (1.95 mL, 11 mmol); stirred at 100° C. for 3 d. The mixture was diluted with ethyl acetate and washed with brine; organic phase was dried, filtered and concentrated in vacuo then chromatographed in 0-10% (2 M NH₃ in MeOH) in EtOAc to give the desired product as a red solid (190 mg, 26%). LCMS: 662.2 (MH⁺).

### (e) 3-[(E)-4-[2-amino-4-carbamoyl-6-[2-(difluoromethoxy)ethoxy]anilino]but-2-enyl]-2-[(2-ethyl-5-methyl-pyrazole-3-carbonyl)amino]thieno[2,3-d]imidazole-5-carboxamide

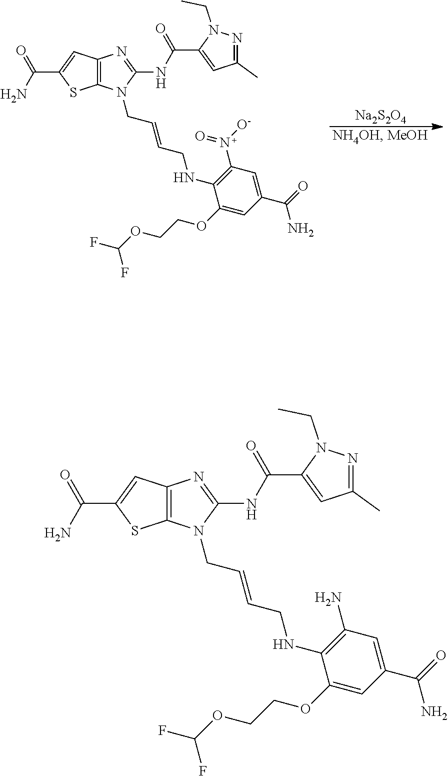

3-[(E)-4-[4-carbamoyl-2-[2-(difluoromethoxy)ethoxy]-6-nitro-anilino]but-2-enyl]-2-[(2-ethyl-5-methyl-pyrazole-3-carbonyl)amino]thieno[2,3-d]imidazole-5-carboxamide (180 mg, 0.27 mmol) was stirred in methanol (6 mL) and ammonium hydroxide (0.5 mL) then treated with sodium dithionite (284 mg, 1.63 mmol) as a solution in water (2 mL); stirred at room temperature for 20 min. The mixture was concentrated in vacuo then stirred in water, ethyl acetate and hexanes; the suspension was filtered to give the desired product (100 mg, 58%). LCMS: 93%, MH⁺.

### (f) 3-[(E)-4-(2-amino-5-carbamoyl-benzimidazol-1-yl)but-2-enyl]-2-[(2-ethyl-5-methyl-pyrazole-3-carbonyl)amino]thieno[2,3-d]imidazole-5-carboxamide Trihydrobromide

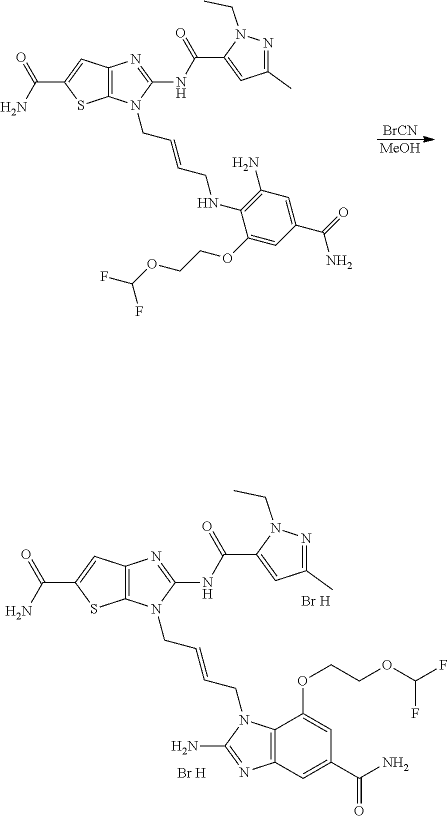

3-[(E)-4-[2-amino-4-carbamoyl-6-[2-(difluoromethoxy)ethoxy]anilino]but-2-enyl]-2-[(2-ethyl-5-methyl-pyrazole-3-carbonyl)amino]thieno[2,3-d]imidazole-5-carboxamide (100 mg, 0.16 mmol) was stirred in methanol (5 mL) and treated with cyanogen bromide (100 mg); stirred for 3 h at room temperature. The mixture was concentrated in vacuo then stirred in diethyl ether; the resulting suspension was filtered to obtain the desired product (123 mg). LCMS: 93%, MH⁺.

### (g) 3-[(E)-4-[5-carbamoyl-7-[2-(difluoromethoxy)ethoxy]-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]thieno[2,3-d]imidazole-5-carboxamide Tetrahydrochloride (Compound N)

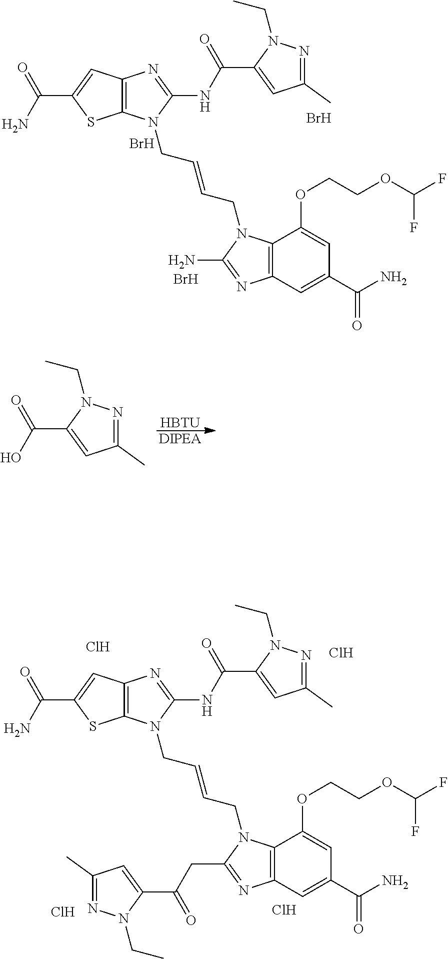

3-[(E)-4-[2-amino-5-carbamoyl-7-[2-(difluoromethoxy)ethoxy]benzimidazol-1-yl]but-2-enyl]-2-[(2-ethyl-5-methyl-pyrazole-3-carbonyl)amino]thieno[2,3-d]imidazole-5-carboxamide trihydrobromide (123 mg, 0.14 mmol) and 2-ethyl-5-methyl-pyrazole-3-carboxylic acid (42 mg, 0.27 mmol) were stirred in DMF (2 mL) then treated with HBTU (104 mg, 0.27 mmol) and DIPEA (120 μL, 0.69 mmol); stirred for 3 h. The mixture was quenched with brine, water and EtOAc; liquid was decanted. The residue was stirred and sonicated with EtOAc and filtered; the filter cake was rinsed with water and diethyl ether. The obtained sticky solid was stirred in methanol treated with HCl, 2 M in diethyl ether (0.3 mL) and concentrated. The residue was stirred in acetone, then concentrated in vacuo to give the desired product as a pale brown solid (40 mg). LCMS (397.1, MH₂²⁺).

### Example 13 Preparation of Compound J

### (a) methyl 6-[[(E)-4-(tert-butoxycarbonylamino)but-2-enyl]amino]-5-nitro-pyridine-3-carboxylate

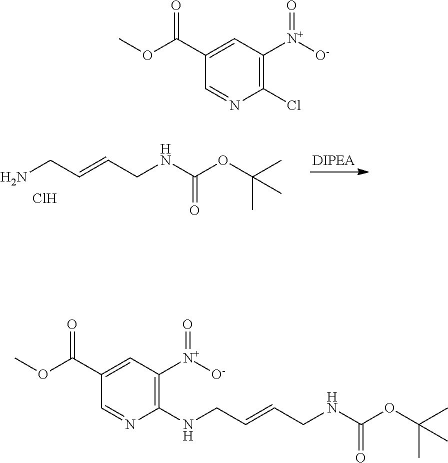 [SMILES: COC(=O)c1cnc(Cl)c([N+](=O)[O-])c1 ; CC(C)(C)OC(=O)NC/C=C/CN ; Cl ; COC(=O)c1cnc(NC/C=C/CNC(=O)OC(C)(C)C)c([N+](=O)[O-])c1]

Methyl 6-chloro-5-nitro-pyridine-3-carboxylate (2.17 g, 10 mmol) and tert-butyl N-[(E)-4-aminobut-2-enyl]carbamate hydrochloride (2.34 g, 10.5 mmol) were stirred in acetonitrile (20 mL) and DIPEA (2.84 g, 22 mmol) at 60° C. for 40 min. The mixture was diluted with water and filtered to collect the desired product (3.33 g, 98%). MS: 367.2 (MH⁺).

### (b) methyl 6-[[(E)-4-aminobut-2-enyl]amino]-5-nitro-pyridine-3-carboxylate Dihydrochloride

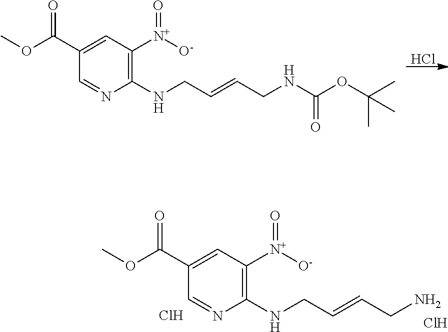

Methyl 6-[[(E)-4-(tert-butoxycarbonylamino)but-2-enyl]amino]-5-nitro-pyridine-3-carboxylate (3.33 g, 9.09 mmol) was stirred in methanol (suspension) then treated with HCl, 4 M in dioxane (22 mL, 87 mmol); solids dissolve, stirred for 30 min. The mixture was concentrated in vacuo then triturated with diethyl ether; filtered to collect the desired product (3.0 g, 97%). MS: 267.1 (MH⁺).

### (c) methyl 6-[[(E)-4-[(5-methoxycarbonyl-3-nitro-2-pyridyl)amino]but-2-enyl]amino]-5-nitro-pyridine-3-carboxylate

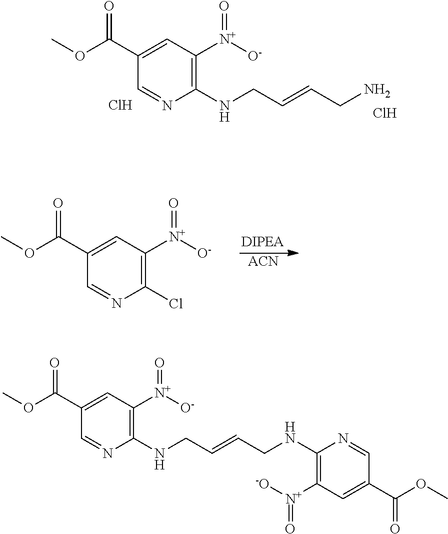

Methyl 6-[[(E)-4-aminobut-2-enyl]amino]-5-nitro-pyridine-3-carboxylate dihydrochloride (1.5 g, 4.42 mmol) and methyl 6-chloro-5-nitro-pyridine-3-carboxylate (1.0 g, 4.64 mmol) were stirred in acetonitrile (15 mL) and DMF (5 mL) then treated with DIPEA (1.54 mL, 8.84 mmol); stirred at 90° C. for 4.5 h. The mixture was cooled to room temperature then diluted with water and diethyl ether; filtered to collect desired product (1.78 g, 90%). MS: 447.1 (MH⁺).

### (d) methyl 2-amino-3-[(E)-4-(2-amino-6-methoxycarbonyl-imidazo[4,5-b]pyridin-3-yl)but-2-enyl]imidazo[4,5-b]pyridine-6-carboxylate

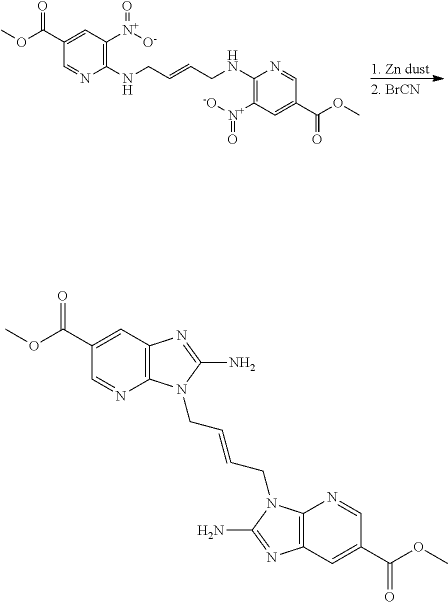

Methyl 6-[[(E)-4-[(5-methoxycarbonyl-3-nitro-2-pyridyl)amino]but-2-enyl]amino]-5-nitro-pyridine-3-carboxylate (500 mg, 1.12 mmol) was stirred in THF (6 mL), methanol (6 mL) and water (5 mL) then treated with zinc dust (700 mg) and NH₄Cl (1.0 g); stirred at 65° C. for 1 h. The mixture was filtered to remove solids (rinsed with methanol); filtrate was concentrated in vacuo. The residue was stirred in methanol then treated with cyanogen bromide (1.12 g, 10.6 mmol); stirred at 65° C. for 2.5 h. The mixture was concentrated in vacuo, stirred with water and diethyl ether then filtered to collect the desired product (388 mg). MS: 437.2 (MH⁺).

### (e) methyl 2-[(2-ethyl-5-methyl-pyrazole-3-carbonyl)amino]-3-[(E)-4-[2-[(2-ethyl-5-methyl-pyrazole-3-carbonyl)amino]-6-methoxycarbonyl-imidazo[4, 5-b]pyridin-3-yl]but-2-enyl]imidazo[4,5-b]pyridine-6-carboxylate (J)

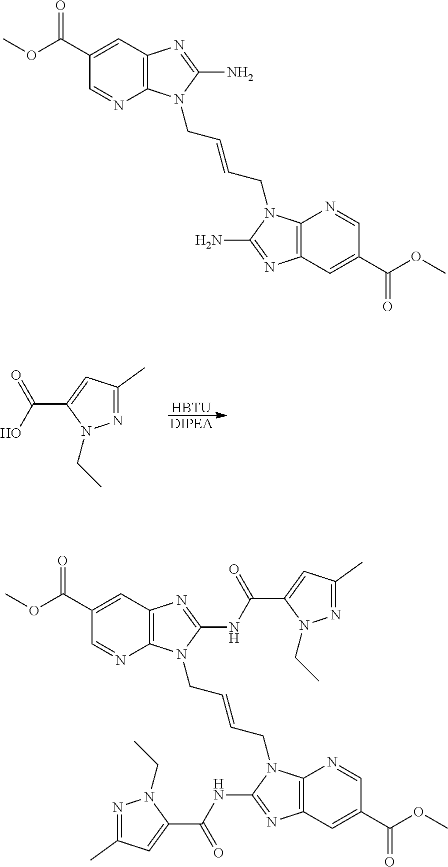

To a stirred solution of methyl 2-amino-3-[(E)-4-(2-amino-6-methoxycarbonyl-imidazo[4,5-b]pyridin-3-yl)but-2-enyl]imidazo[4, 5-b]pyridine-6-carboxylate (388 mg, 0.89 mmol), 2-ethyl-5-methyl-pyrazole-3-carboxylic acid (548 mg, 3.56 mmol) in DMF (7.5 mL) was added HBTU (1.69 g, 4.45 mmol) followed by DIPEA (619 μL, 3.56 mmol); stirred at 40° C. overnight. The mixture was filtered, rinsed with methanol to give the desired product as a white solid (430 mg, 68%). MS: 709.3 (MH⁺).

### Example 14 Preparation of Compound K

### a) 3-[(E)-4-[2-[3-[tert-butyl(dimethyl)silyl]oxypropoxy]-4-carbamoyl-6-nitro-anilino]but-2-enyl]-2-[(2-ethyl-5-methyl-pyrazole-3-carbonyl)amino]thieno[2,3-d]imidazole-5-carboxamide

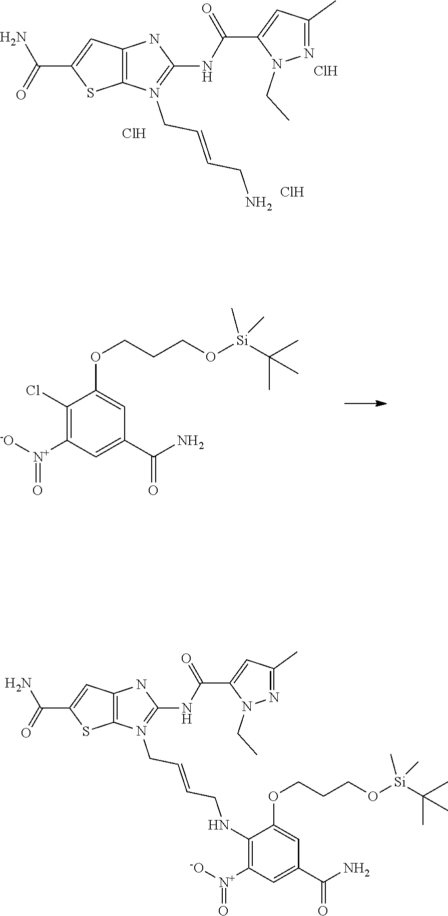

To a stirred suspension of 3-[(E)-4-aminobut-2-enyl]-2-[(2-ethyl-5-methyl-pyrazole-3-carbonyl)amino]thieno[2,3-d]imidazole-5-carboxamide trihydrochloride (720 mg, 1.45 mmol) and 3-[3-[tert-butyl(dimethyl)silyl]oxypropoxy]-4-chloro-5-nitro-benzamide (554 mg, 1.42 mmol) in n-propanol (10 mL) was added DIPEA (1.3 mL, 7.5 mmol). The mixture was stirred at 110° C. for 3 d. The mixture was diluted with ethyl acetate and washed with brine (1×), water:brine (1×) and brine (1×). The organic phase was dried, filtered and concentrated in vacuo then chromatographed in 0-50% THF in ethyl acetate; product containing fractions were concentrated in vacuo giving the desired product as a yellow solid (540 mg). MS: 370.6 (MH₂²⁺).

### b) 3-[(E)-4-[2-amino-6-[3-[tert-butyl(dimethyl)silyl]oxypropoxy]-4-carbamoyl-anilino]but-2-enyl]-2-[(2-ethyl-5-methyl-pyrazole-3-carbonyl)amino]thieno[2,3-d]imidazole-5-carboxamide

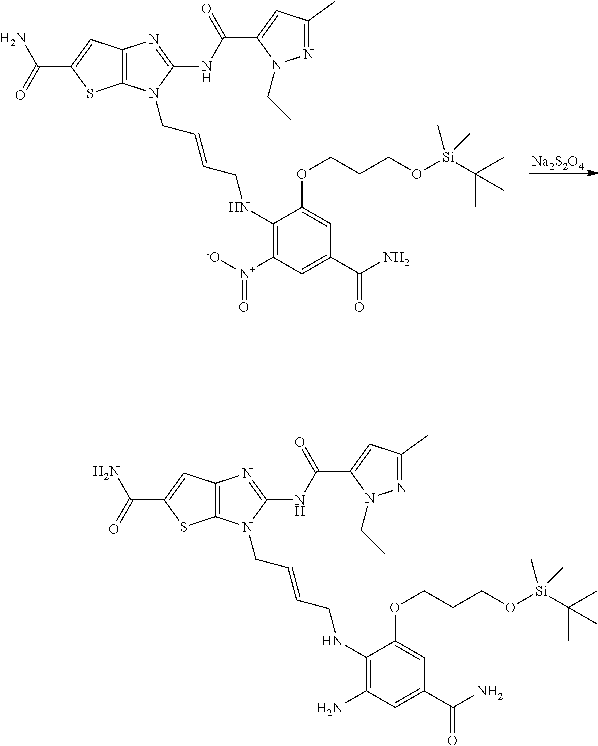

3-[(E)-4-[2-[3-[tert-butyl(dimethyl)silyl]oxypropoxy]-4-carbamoyl-6-nitro-anilino]but-2-enyl]-2-[(2-ethyl-5-methyl-pyrazole-3-carbonyl)amino]thieno[2,3-d]imidazole-5-carboxamide (540 mg, 0.73 mmol) was stirred in methanol (15 mL) and ammonium hydroxide (1.5 mL); treated with sodium dithionite (762 mg, 4.38 mmol) as a solution in water (6 mL) then stirred for 20 min. Mixture was concentrated in vacuo, diluted with diethyl ether and water then filtered to collect the desired product, used directly in the subsequent step (327 mg, 63%). MS: 355.6 (MH₂²⁺).

### c) 3-[(E)-4-[2-amino-7-[3-[tert-butyl(dimethyl)silyl]oxypropoxy]-5-carbamoyl-benzimidazol-1-yl]but-2-enyl]-2-[(2-ethyl-5-methyl-pyrazole-3-carbonyl)amino]thieno[2,3-d]imidazole-5-carboxamide

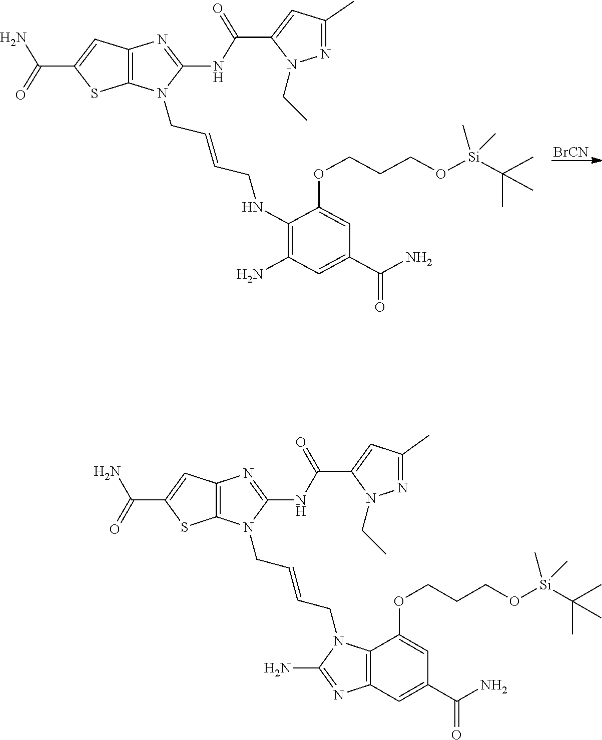

3-[(E)-4-[2-amino-6-[3-[tert-butyl(dimethyl)silyl]oxypropoxy]-4-carbamoyl-anilino]but-2-enyl]-2-[(2-ethyl-5-methyl-pyrazole-3-carbonyl)amino]thieno[2,3-d]imidazole-5-carboxamide (327 mg, 0.46 mmol) was stirred in methanol and treated with cyanogen bromide (300 mg, 2.83 mmol); stirred for 1.5 h. The mixture was filtered to remove solids, filtrate was concentrated in vacuo then stirred with water. The resulting suspension was filtered, solid material was washed with water and diethyl ether to give the crude desired product (250 mg), used directly in the subsequent reaction. MS: 368.2 (MH₂²⁺).

### d) 3-[(E)-4-[7-[3-[tert-butyl(dimethyl)silyl]oxypropoxy]-5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]thieno[2,3-d]imidazole-5-carboxamide

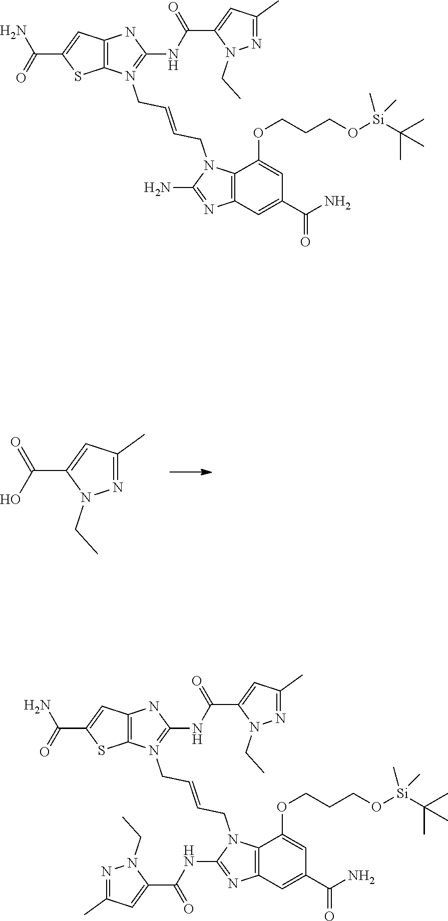

3-[(E)-4-[2-amino-7-[3-[tert-butyl(dimethyl)silyl]oxypropoxy]-5-carbamoyl-benzimidazol-1-yl]but-2-enyl]-2-[(2-ethyl-5-methyl-pyrazole-3-carbonyl)amino]thieno[2,3-d]imidazole-5-carboxamide (250 mg, 0.34 mmol) and 2-ethyl-5-methyl-pyrazole-3-carboxylic acid (104 mg, 0.68 mmol) were stirred in DMF (5 mL); the mixture was treated with HBTU 9258 mg, 0.68 mmol) followed by DIPEA (118 μL, 0.68 mmol) and stirred for 6 h. The mixture was diluted with ethyl acetate then washed with NaHCO₃(1×) and brine (3×). The organic phase was dried, filtered and concentrated in vacuo then triturated with hexanes to give the desired product (200 mg) used directly in the subsequent reaction. MS: 436.2 (MH₂²⁺).

### e) 3-[(E)-4-[5-carbamoyl-2-[(2-ethyl-5-methyl-pyrazole-3-carbonyl)amino]-7-(3-hydroxypropoxy)benzimidazol-1-yl]but-2-enyl]-2-[(2-ethyl-5-methyl-pyrazole-3-carbonyl)amino]thieno[2,3-d]imidazole-5-carboxamide (K)

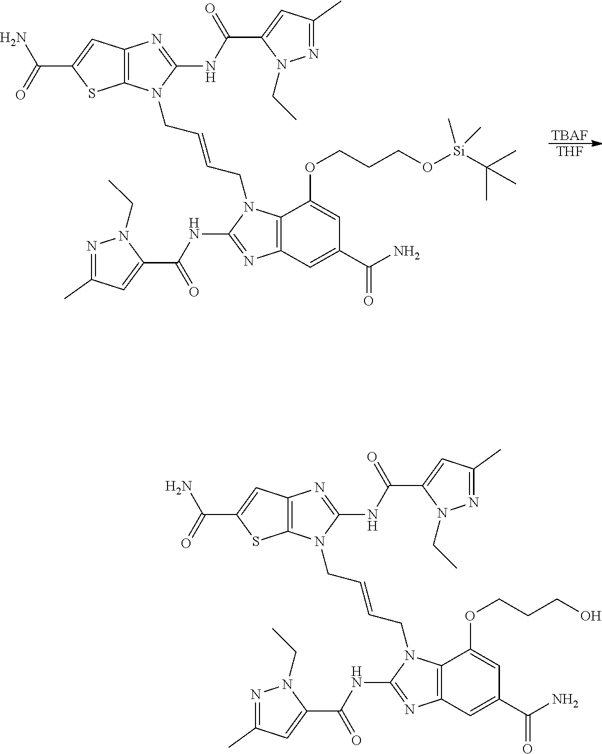

3-[(E)-4-[7-[3-[tert-butyl(dimethyl)silyl]oxypropoxy]-5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]thieno[2,3-d]imidazole-5-carboxamide (200 mg, 0.23 mmol) was stirred in THF (5 mL) then treated with TBAF (1 M in THF, 460 μL, 0.46 mmol); stirred for 7 h. The mixture was concentrated in vacuo to give crude product, used directly in the subsequent reaction. MS: 378.7 (MH₂²⁺).

### Example 15 Preparation of Compound R

### a) 3-[(E)-4-[2-[3-[tert-butyl(dimethyl)silyl]oxypropoxy]-4-carbamoyl-6-nitro-anilino]but-2-enyl]-2-[(2-ethyl-5-methyl-pyrazole-3-carbonyl)amino]imidazo[4,5-b]pyridine-6-carboxamide

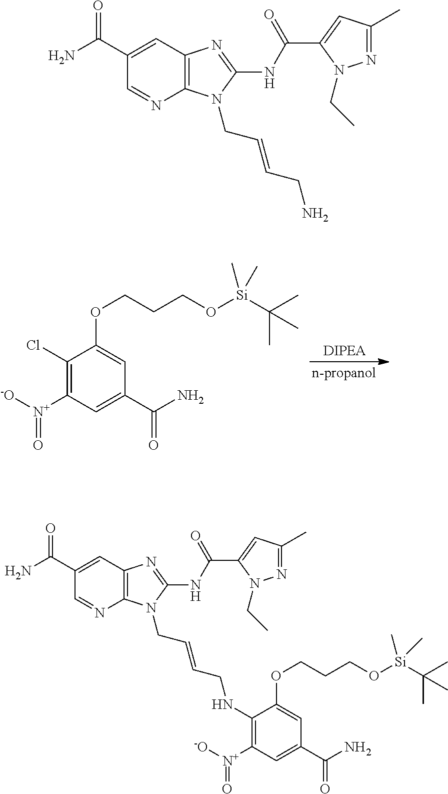

3-[(E)-4-aminobut-2-enyl]-2-[(2-ethyl-5-methyl-pyrazole-3-carbonyl)amino]imidazo[4,5-b]pyridine-6-carboxamide (2.53 g, 5.14 mmol) and 3-[3-[tert-butyl(dimethyl)silyl]oxypropoxy]-4-chloro-5-nitro-benzamide (2.0 g, 5.14 mmol) were stirred in n-propanol (25 mL) and DIPEA (5.37 mL, 30.8 mmol); the flask was sealed and the mixture was stirred for 3 d at 120° C. The mixture was diluted with ethyl acetate and washed with brine. The organic phase was dried, filtered and concentrated in vacuo then chromatographed in 0-20% methanol in ethyl acetate. The product containing fractions were concentrated in vacuo then stirred in diethyl ether and hexanes; filtered to collect the desired product as an orange solid (3.1 g, 83%). MS: 735.3 (MH⁺).

### b) 3-[(E)-4-[2-amino-6-[3-[tert-butyl(dimethyl)silyl]oxypropoxy]-4-carbamoyl-anilino]but-2-enyl]-2-[(2-ethyl-5-methyl-pyrazole-3-carbonyl)amino]imidazo[4,5-b]pyridine-6-carboxamide

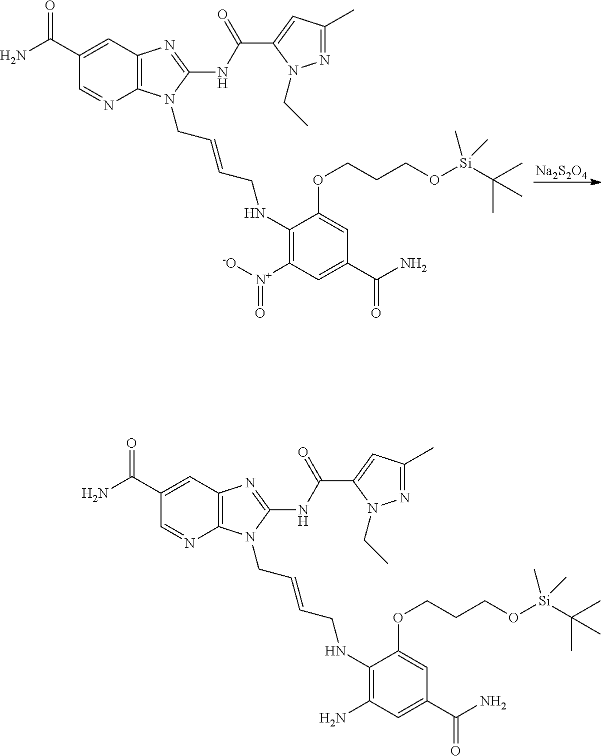

To a stirred suspension of 3-[(E)-4-[2-[3-[tert-butyl(dimethyl)silyl]oxypropoxy]-4-carbamoyl-6-nitro-anilino]but-2-enyl]-2-[(2-ethyl-5-methyl-pyrazole-3-carbonyl)amino]imidazo[4,5-b]pyridine-6-carboxamide (2.95 g, 4.0 mmol) in methanol (50 mL) was added ammonium hydroxide (8 mL). The resulting clear (red) solution was treated with sodium dithionite (4.2 g, 24 mmol) as a solution in water (30 mL); reaction turns from a red solution to a yellow suspension. The mixture was diluted with water and extracted with ethyl acetate (2×). The organic phase was concentrated in vacuo to give the crude product, used directly in the subsequent reaction. MS: 705.4 (MH⁺).

### c) 3-[(E)-4-[7-[3-[tert-butyl(dimethyl)silyl]oxypropoxy]-5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]imidazo[4,5-b]pyridine-6-carboxamide

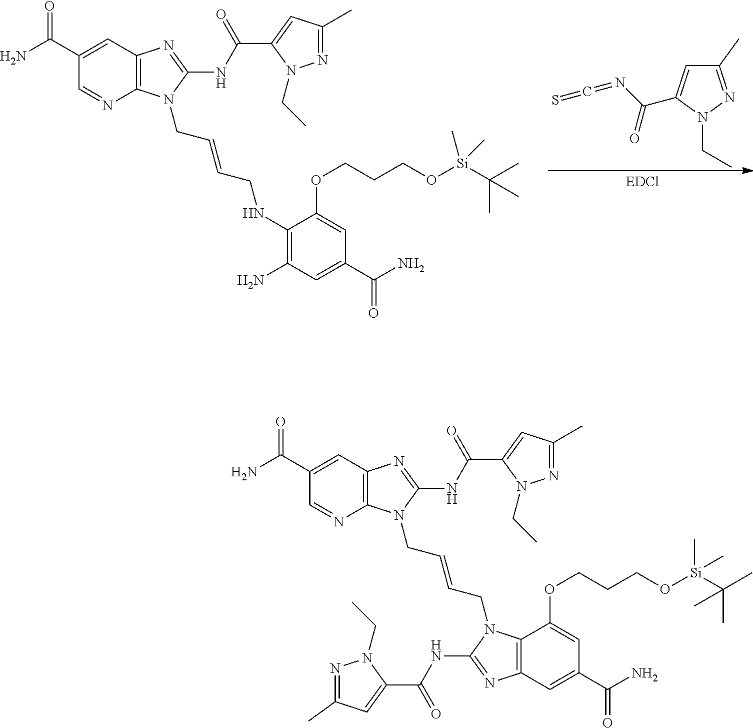 [SMILES: CCn1nc(C)cc1C(=O)Nc1nc2cc(C(N)=O)cnc2n1C/C=C/CNc1c(N)cc(C(N)=O)cc1OCCCO[Si](C)(C)C(C)(C)C ; CCn1nc(C)cc1C(=O)N=C=S ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO[Si](C)(C)C(C)(C)C)c21]

To a stirred solution of 3-[(E)-4-[2-amino-6-[3-[tert-butyl(dimethyl)silyl]oxypropoxy]-4-carbamoyl-anilino]but-2-enyl]-2-[(2-ethyl-5-methyl-pyrazole-3-carbonyl)amino]imidazo[4,5-b]pyridine-6-carboxamide (1.03 g, 1.46 mmol) in DMF (10 mL) cooled to 0° C. was added 2-ethyl-5-methyl-pyrazole-3-carbonyl isothiocyanate (286 mg, 1.46 mmol) slowly. The mixture was stirred for 1 h then treated with 1-ethyl-3-(3-dimethylaminopropyl)carbodiimide hydrochloride (420 mg, 2.19 mmol) followed by DIPEA (761 μL, 4.38 mmol); stirred at room temperature overnight. The mixture was diluted with brine and extracted with ethyl acetate. The organic phase was dried, filtered and concentrated in vacuo then chromatographed in 0-7.5% methanol in DCM. The product containing fractions were concentrated in vacuo and stirred in diethyl ether; filtered to collect the desired product as an off-white solid (1.06 g, 84%). MS: 866.4 (MH⁺).

### d) 3-[(E)-4-[7-[3-[tert-butyl(dimethyl)silyl]oxypropoxy]-5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]imidazo[4,5-b]pyridine-6-carboxamide (R)

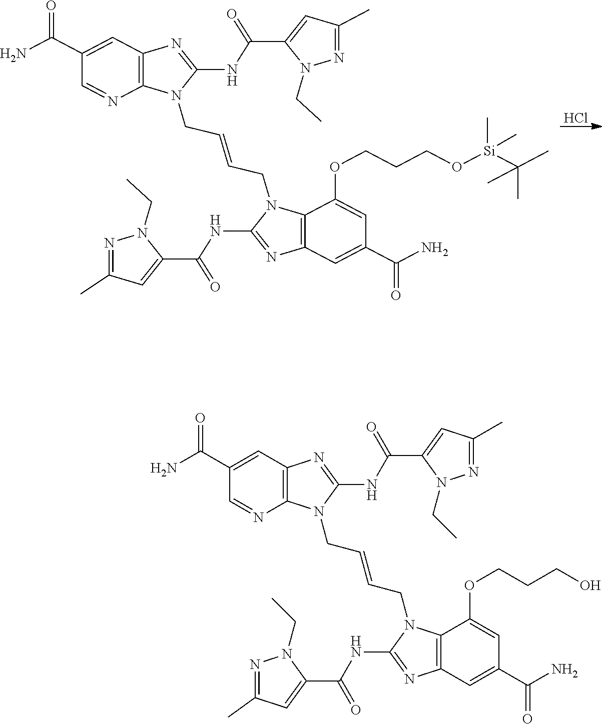 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO[Si](C)(C)C(C)(C)C)c21 ; Cl ; CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCO)c21]

To a stirred solution of 3-[(E)-4-[7-[3-[tert-butyl(dimethyl)silyl]oxypropoxy]-5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]imidazo[4,5-b]pyridine-6-carboxamide (1.06 g, 1.2 mmol) was added HCl, 4 M in dioxane (2 mL, 8 mmol). The resulting mixture was stirred for 20 min, concentrated in vacuo then stirred in diethyl ether. The suspension was filtered to collect the desired product as a hydrochloride salt (801 mg, 87%). MS: 752.3 (MH⁺).

### Example 16 Preparation of Compound Q

### a) 3-[6-carbamoyl-3-[(E)-4-[5-carbamoyl-2-[(2-ethyl-5-methyl-pyrazole-3-carbonyl)amino]thieno[2,3-d]imidazol-3-yl]but-2-enyl]-2-[(2-ethyl-5-methyl-pyrazole-3-carbonyl)amino]benzimidazol-4-yl]oxypropyl methanesulfonate

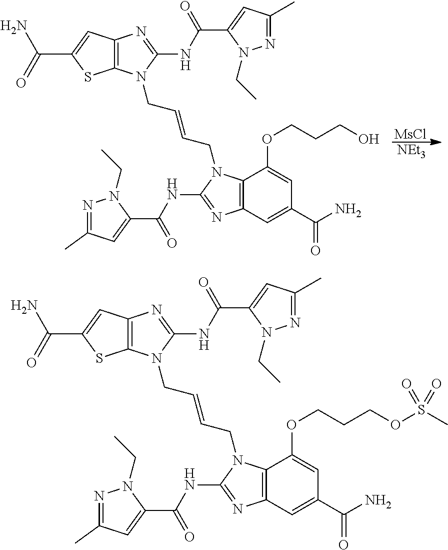

3-[(E)-4-[5-carbamoyl-2-[(2-ethyl-5-methyl-pyrazole-3-carbonyl)amino]-7-(3-hydroxypropoxy)benzimidazol-1-yl]but-2-enyl]-2-[(2-ethyl-5-methyl-pyrazole-3-carbonyl)amino]thieno[2,3-d]imidazole-5-carboxamide (190 mg, 0.23 mmol) was stirred in THF (20 mL) and treated with triethylamine (66 μL, 0.47 mmol) followed by methanesulfonyl chloride (28 μL, 0.36 mmol); more triethylamine and methanesulfonyl chloride were added until SM was consumed. The mixture was filtered to remove solids and used directly in the subsequent reaction. MS: 417.7 (MH₂²⁺).

The following compound was made in a similar fashion:

### 3-[6-carbamoyl-3-[(E)-4-[6-carbamoyl-2-[(2-ethyl-5-methyl-pyrazole-3-carbonyl)amino]imidazo[4,5-b]pyridin-3-yl]but-2-enyl]-2-[(2-ethyl-5-methyl-pyrazole-3-carbonyl)amino]benzimidazol-4-yl]oxypropyl methanesulfonate

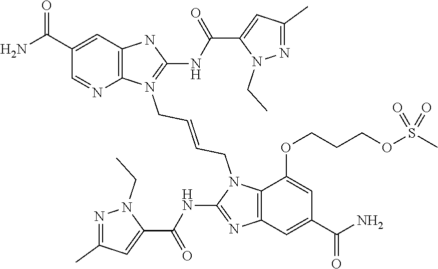

MS: 830.3 (MH⁺).

### b) 3-[(E)-4-[5-carbamoyl-2-[(2-ethyl-5-methyl-pyrazole-3-carbonyl)amino]-7-[3-(methylamino)propoxy]benzimidazol-1-yl]but-2-enyl]-2-[(2-ethyl-5-methyl-pyrazole-3-carbonyl)amino]thieno[2,3-d]imidazole-5-carboxamide (Compound Q)

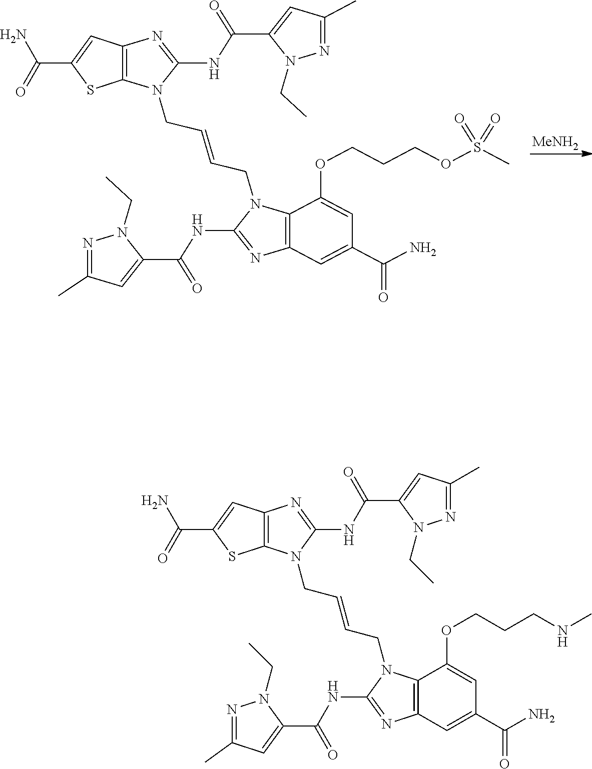 [SMILES: "MeNH2" ; CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCOS(C)(=O)=O)c21 ; CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCNC)c21]

3-[6-carbamoyl-3-[(E)-4-[5-carbamoyl-2-[(2-ethyl-5-methyl-pyrazole-3-carbonyl)amino]thieno[2,3-d]imidazol-3-yl]but-2-enyl]-2-[(2-ethyl-5-methyl-pyrazole-3-carbonyl)amino]benzimidazol-4-yl]oxypropyl methanesulfonate (0.11 mmol) was stirred in methylamine (2 M in THF, 10 mL) at 65° C. overnight. The mixture was concentrated in vacuo; the residue was stirred in diethyl ether and water. The emulsion was filtered to collect the desired product (25 mg) as a beige solid. MS: 385.3 (MH₂²⁺).

The following compounds were made in a similar fashion:

### 3-[(E)-4-[5-carbamoyl-7-[3-(dimethylamino)propoxy]-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]thieno[2,3-d]imidazole-5-carboxamide (Compound O)

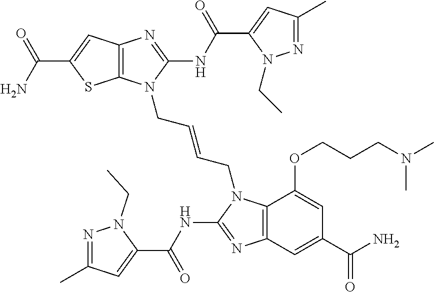 [SMILES: CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCN(C)C)c21]

LCMS: 392.68 (MH₂²⁺).

### 3-[(E)-4-[5-carbamoyl-7-[3-(dimethylamino)propoxy]-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]imidazo[4,5-b]pyridine-6-carboxamide (Compound P)

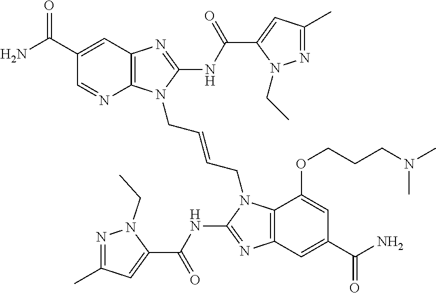 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN(C)C)c21]

MS: 390.2 (MH₂²⁺).

### 3-[(E)-4-[5-carbamoyl-2-[(2-ethyl-5-methyl-pyrazole-3-carbonyl)amino]-7-[3-(methylamino)propoxy]benzimidazol-1-yl]but-2-enyl]-2-[(2-ethyl-5-methyl-pyrazole-3-carbonyl)amino]imidazo[4,5-b]pyridine-6-carboxamide (Compound S)

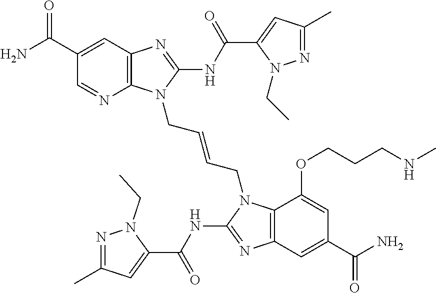 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNC)c21]

MS: 383.2 (MH₂²⁺).

### 3-[(E)-4-[5-carbamoyl-2-[(2-ethyl-5-methyl-pyrazole-3-carbonyl)amino]-7-(3-morpholinopropoxy)benzimidazol-1-yl]but-2-enyl]-2-[(2-ethyl-5-methyl-pyrazole-3-carbonyl)amino]imidazo[4,5-b]pyridine-6-carboxamide (Compound T)

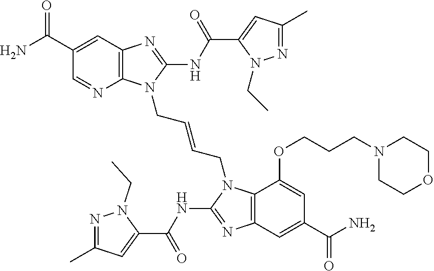 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21]

MS: 411.2 (MH₂²⁺).

### 3-[(E)-4-[5-carbamoyl-7-[3-[2-(difluoromethoxy)ethylamino]propoxy]-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]imidazo[4, 5-b]pyridine-6-carboxamide (Compound U)

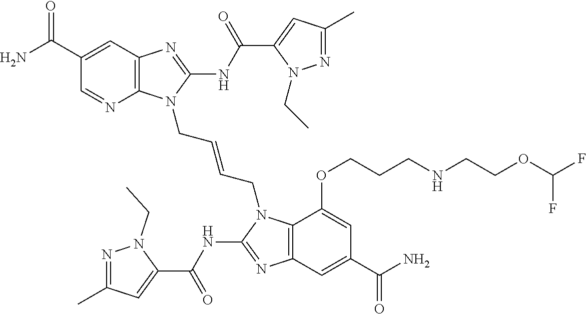 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNCCOC(F)F)c21]

MS: 845.3 (MH⁺), 423.2 (MH₂²⁺).

### 3-[(E)-4-[5-carbamoyl-2-[(2-ethyl-5-methyl-pyrazole-3-carbonyl)amino]-7-[3-[2-methoxyethyl(methyl)amino]propoxy]benzimidazol-1-yl]but-2-enyl]-2-[(2-ethyl-5-methyl-pyrazole-3-carbonyl)amino]imidazo[4, 5-b]pyridine-6-carboxamide (Compound V)

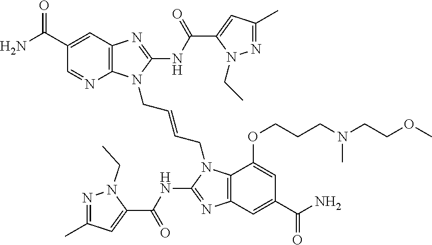 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN(C)CCOC)c21]

MS: 823.3 (MH⁺), 412.3 (MH₂²⁺).

### 3-[(E)-4-[5-carbamoyl-2-[(2-ethyl-5-methyl-pyrazole-3-carbonyl)amino]-7-[3-(2,2,3,3,5,5,6,6-octadeuteriomorpholin-4-yl)propoxy]benzimidazol-1-yl]but-2-enyl]-2-[(2-ethyl-5-methyl-pyrazole-3-carbonyl)amino]imidazo[4, 5-b]pyridine-6-carboxamide (Compound W)

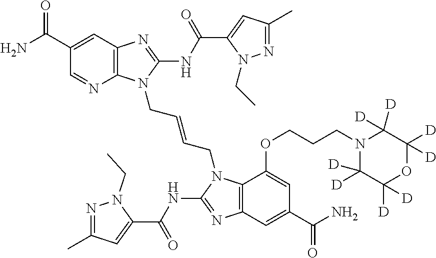 [SMILES: [2H]C1([2H])OC([2H])([2H])C([2H])([2H])N(CCCOc2cc(C(N)=O)cc3nc(NC(=O)c4cc(C)nn4CC)n(C/C=C/Cn4c(NC(=O)c5cc(C)nn5CC)nc5cc(C(N)=O)cnc54)c23)C1([2H])[2H]]

MS: 829.3 (MH⁺), 415.3 (MH₂²⁺).

### 3-[(E)-4-[7-[3-(4-acetylpiperazin-1-yl)propoxy]-5-carbamoyl-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]imidazo[4,5-b]pyridine-6-carboxamide (Compound X)

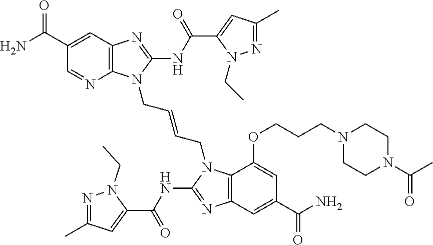 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCN(C(C)=O)CC3)c21]

MS: 862.3 (MH⁺), 431.7 (MH₂²⁺).

### 3-[(E)-4-[5-carbamoyl-7-[3-[(2S)-2-(difluoromethoxymethyl)pyrrolidin-1-yl]propoxy]-2-[(2-ethyl-5-methyl-pyrazole-3-carbonyl)amino]benzimidazol-1-yl]but-2-enyl]-2-[(2-ethyl-5-methyl-pyrazole-3-carbonyl)amino]imidazo[4,5-b]pyridine-6-carboxamide (Compound Y)

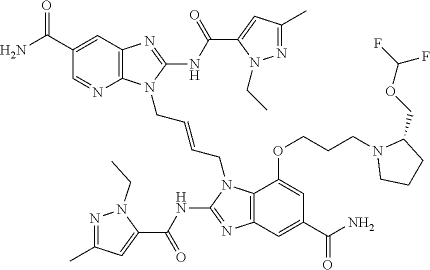 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC[C@H]3COC(F)F)c21]

MS: 885.3 (MH⁺), 443.3 (MH₂²⁺).

### 3-[(E)-4-[5-carbamoyl-2-[(2-ethyl-5-methyl-pyrazole-3-carbonyl)amino]-7-[3-(1,4-oxazepan-4-yl)propoxy]benzimidazol-1-yl]but-2-enyl]-2-[(2-ethyl-5-methyl-pyrazole-3-carbonyl)amino]imidazo[4,5-b]pyridine-6-carboxamide Compound Z)

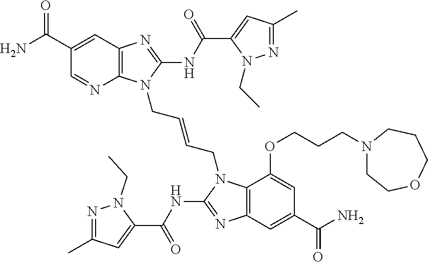 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCCOCC3)c21]

MS: 835.3 (MH⁺), 418.3 (MH₂²⁺).

### Biology:

The agonist activity of the compounds of this application were determined using cell based assays that measure reporter activities driven by STING activation in engineered culture cell lines or secreted IFN-beta induced by STING activation from primary cells.

Cellular Functional Assay I (Engineered Culture Cell Lines with Reporter):

Human embryonic kidney cells (HEK-293T)-Dual Null™ (InvivoGen) were transfected with plasmids expressing three human STING alleles (pUNO1-hSTING-WT™, pUNO1-hSTING-H232™, and pUNO1-hSTING-WT-HAQ™, InvivoGen), mouse STING (pUNO1-mSTING™, InvivoGen), and vector only (pUNO1™, InvivoGen) as control, respectively. The parent HEK-293T-Dual Null™ cells are known to have a non-functional STING and are engineered to stably express an IFN-stimulated response elements (ISRE) inducible SEAP (secreted embryonic alkaline phosphatase) as a reporter. STING agonist stimulation was assessed by monitoring secreted SEAP activities in supernatant. Stable transfected cells were generated in cell culture with the selection antibiotic blasticidin. Cells were seeded at a density of 20,000 cells/well in 50 ul media in a flat bottom 96-well plate. The seeded cells permitted to attach to the plate overnight by incubating them in DMEM™ (ATCC) at 37° C. with 5% CO₂. The next day, serially diluted test compound (50 ul) or DMSO (control) were added into each well. Treated cells were incubate for 4 hours at 37° C. with 5% CO₂. After 4 hours of incubation, the supernatant was carefully removed and fresh media containing blasticidin (100 ul) was added into each well. The plate was incubated for an additional 20 hours before the supernatant (95 ul) was harvested. The recovered supernatants were tested for SEAP activity using the InvivoGen QuantiBlue™ kit.

Tested compounds exhibited EC50 values in the range of uM level to pM level as shown below in Table 1. The compounds of the application were found to be agonists of one or more human and/or mouse STING alleles (hWT, hH232, hHAQ, hAQ, hQ and/or mWT). The results are presented as EC₅₀ in Table 1. The letter “A” indicates an EC₅₀<1 nM; “B” indicates an EC₅₀>1 nM but <500 nM; and “C” indicates an EC₅₀>500 nM. Examples I(a), I(c) and I(n) displayed EC₅₀'s at pM or nM level for three human STING alleles (WT, REF (also called H232), and HAQ) and mouse STING.

TABLE 1

| Compound I.D | hSTING-wt | hSTING-H232 | hSTING-HAQ | hSTING-AQ | hSTING-Q | mSTING-wt |
|---|---|---|---|---|---|---|
| A | A | A | B | B | B | A |
| B | A | A | B | B | B | B |
| C | B | B | C | B | B | C |
| D | C | C | C | C | C | C |
| E | C | C | C | C | C | C |
| F | C | C | C | C | C | C |
| G | C | C | C | C | C | C |
| H | C | C | C | C | C | C |
| L | B | B | C | C | B | B |
| M | B | B | C | C | B | B |
| N | A | A | C | C | B | B |
| O | B | B | | | | |
| P | B | B | B | B | B | B |
| Q | B | B | | | | |
| R | B | B | B | B | B | B |
| S | B | B | B | B | B | B |
| T | B | B | B | B | B | B |
| U | B | B | B | B | B | B |
| V | B | B | B | B | B | B |
| W | B | B | B | B | B | B |
| X | B | B | B | B | B | B |
| Y | A | A | B | B | B | B |
| Z | B | B | B | B | B | B |
| 2′3-cGAMP | C | C | C | C | C | C |

Cellular Functional Assay II (Measure IFN-β from Primary Cells):

Activation of STING in primary cells was determined by measuring levels of secreted IFN-β from human monocyte-derived macrophages (h-MDMs)/dendritic cells (h-DCs) and/or mouse dendritic cells (m-DCs) after treatment with A.

Human CD14+ cells were first isolated from human peripheral blood mononuclear cells (PBMCs) with Stemcell Easysep Monocyte Isolation™ kit. Human CD14+ cells were kept in conditioned media for 7-12 days to generate h-MDMs and h-DCs before use. After harvesting from C57BL/6 mouse femurs, bone marrow cells were culture in conditioned media for 7 days to generate m-DCs before use.

The Culture conditions for the various types of cells whereas noted in Table 2:

TABLE 2
For compound treatment, cells were suspended at a cell density of

| | Human | | |
|---|---|---|---|
| cell | MDM | Human DC | Mouse DC |
| Media | Xvivo 15 + | RPMI + 10% FBS + | Hyclone RPMI + 10% |
| | 20 ng/mL | 40 ng/ml hGM-CSF + | FBS + NaPyr + Pen/Strep + |
| | hM-CSF | 40 ng/ml hIL-4 | B2Me + 20 ng/mL |
| | | | mGM-CSF |

0.5×10\hat{ }6 cells in 50 ul buffer (50 mM HEPES, pH7.0; 100 mM KCl; 3 mM MgCl₂, 0.1 mM DTT; 85 mM Sucrose; 0.2% BSA, 1 mM ATP) in 96-well round bottom plate. Compound (formulated in DMSO) or DMSO (control) were added into each well and the plate incubate at 37° C. for 30 minutes. Cells were washed twice with 100 uL of fresh media and incubated at 37° C. for an additional 6 hours. After 6 hour of incubation, the supernatant was collected and a Cytometry Bead Assay™ (CBA, BioLegend) was used to measure IFN-3 levels.

Exemplary compounds of the application were found to be agonists of human and mouse dendritic cells (DC) and macrophages. The results are presented in Table 3.

TABLE 3

| | | | | | | | | 2′3′-cGAMP(IFN-β | | |
|---|---|---|---|---|---|---|---|---|---|---|
| | | A (IFN-β level (pg/ml)) | | | B (IFN-β level (pg/ml)) | | | level (pg/ml)) | | |
| Concentration | | h-MDM | h-DC | m-DC | h-DC | m-DC | h-DC | h-MDM | h-DC | m-DC |
| 10 | uM | >1,000 | >15,000 | >50,000 | >1,000 | >60,000 | >1,000 | <50 | <50 | <3,000 |
| 1 | uM | >1,000 | N/A | >10,000 | >1,000 | N/A | >1,000 | <20 | N/A | <1,000 |
| 0.1 | uM | >1,000 | >5,000 | >100 | >800 | N/A | >800 | <10 | <10 | <300 |

The in vivo efficacy of exemplary compounds of the application were determined in immunocompetent syngeneic mouse models. In some embodiments, exemplary compounds of the application were injected directly into a primary tumor and a tumoricidal effect was observed at the site of injection.

Cellular Functional Assay III (IFN-β Production from Human PBMCs):

Frozen human PBMCs (BioIVT Inc.) were thawed first in a 37° C. water bath, then washed with RPMI/10% FBS/2 mM EDTA medium twice before establishing a viable cell count by Trypan Blue. The final cell density for each donor was 5×10⁶ cells per well with RPMI/10% FBS medium in a 96 well u-bottom plate.

Human PBMCs were then incubated with testing compounds with indicated concentrations and control for 4 hours at 37° C. in cell culture incubator. After incubation, supernatant was collected and used for measuring induced cytokine level by cytometric bead array analysis. Results are presented in Table 4 as EC₅₀s for producing an IFN-3 response: The letter “A” indicates an EC₅₀<100 nM; “B” indicates an EC₅₀>100 nM but <500 nM; and “C” indicates and EC₅₀>500 nM.

TABLE 4

| | Potency (IFN-β production) in Human PBMCs (EC50s) | | | |
|---|---|---|---|---|
| Compound | Donor 1 | Donor 2 | Donor 3 | Donor 4 |
| T | B | A | A | B |
| W | B | B | A | B |
| X | B | B | B | B |
| Y | A | A | A | A |

### In Vivo Syngeneic A20 Mouse Tumor Model:

The in vivo efficacy of exemplary compound I(c) was evaluated in a murine A20 model of B cell lymphoma. A20 cells were maintained in RPMI1640/10% FBS. Approximately 4×10⁶ A20 cells were injected subcutaneously in 0.1 mL PBS into the right flanks of immunocompetent female Balb/c mice. Tumors were permitted to grow for six days after initial inoculation before the animals were segregated into treatment group. Study groups were treated with 25 μg of B formulated in an appropriate vehicle. Exemplary compound B was dosed intratumorally at the proximal tumor (right flank) on days 6, 8, and 11 of the study. Only animals in good health were used for dosing. Since the effects of the test article on the animals were not known, animals were closely observed for 1 h following dosing and at each tumor measurement time point over the course of the study. Any adverse reactions to the administration of the test material were noted and tabulated. Mice exhibiting moribund behavior or experiencing a severe level of pain were humanely euthanized using isoflurane anesthesia. Systemic toxicity of the treatments was evaluated by body weight change. Tumor volume was measured every 2 days using standard caliper measurement techniques.

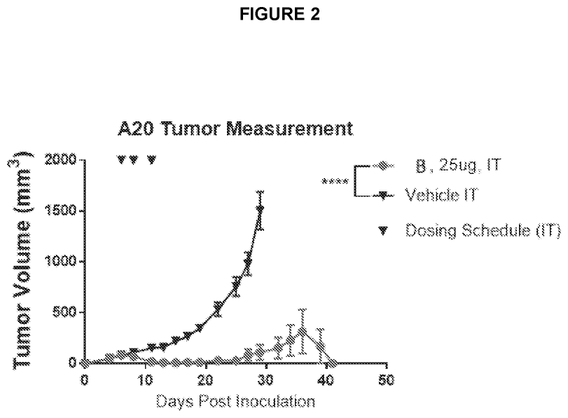

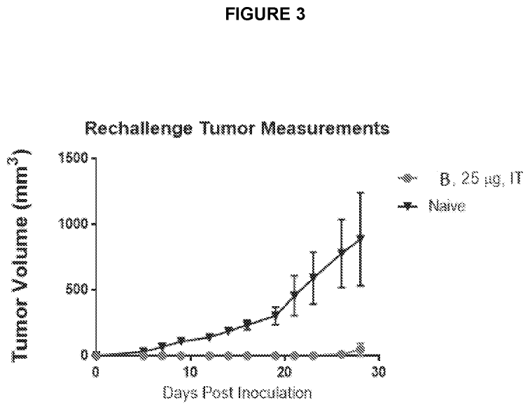

Exemplary compound B exhibited significant tumoricidal properties at the injected tumor with complete tumor regression being observed in 80% of animals receiving 25 μg of B (FIG. 2, ****=p<0.0001). Furthermore, no dose related toxicity, as measured by body weight loss, was observed in the 25 μg dose group. Tumor free mice from B 25 μg treated group were re-challenged with A20 cells. Naïve Balb/c mice were inoculated with A20 cells as control. Tumor re-growth was monitored for 30 days (FIG. 3).

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

What is claimed is:

1. A compound of Formula I: 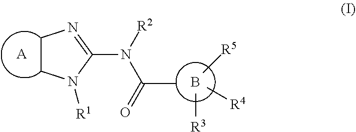
wherein:
Ring A is a 5 or 6-membered heteroaromatic ring comprising 1 to 3 heteroatoms selected from O, N and S that is substituted with at least one R⁶ and optionally substituted with one or two additional substituents independently selected from halo, C₁₋₆alkyl, C₂₋₆alkenyl, C₂₋₆alkynyl, OR^{a}, NR^{a}R^{b}, C₁₋₆alkyleneOR^{a}, C₁₋₆alkyleneNR^{a}R^{b}, OC₁₋₆alkyleneOR^{a}, OC₁₋₆alkyleneNR^{a}R^{b}, C₂₋₆alkenyleneOR^{a}, C₂₋₆alkenyleneNR^{a}R^{b}, OC₂₋₆alkenyleneOR^{a}, OC₂₋₆alkenyleneNR^{a}R^{b}, C₂₋₆alkynyleneOR^{a}, C₂₋₆alkynyleneNR^{a}R^{b}, OC₂₋₆alkynyleneOR^{a} and OC₂₋₆alkynyleneNR^{a}R^{b};
R^{a} and R^{b} are independently selected from H, C₁₋₆alkyl, C₁₋₆alkyleneOC₁₋₆alkyl, C₂₋₆alkenyleneOC₁₋₆alkyl, and C₂₋₆alkynyleneOC₁₋₆alkyl, or
R^{a} and R^{b} are joined to form, together with the atoms therebetween, a 3-10 membered saturated or unsaturated ring, optionally containing one additional heteroatom selected from NR^{s}, O S, SO and SO₂, and optionally substituted with one or more of D, OH, NH₂, CHO, CO₂H, CONH₂, C₁₋₆alkyl, C₁₋₆alkenyl, C₁₋₆alkynyl C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R^{s} is selected from H, D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R¹ is selected from C₁₋₁₀alkyleneR⁷, C₂₋₁₀alkenyleneR⁷, C₂₋₁₀alkynyleneR⁷, C₃₋₁₀cycloalkyl, 4-10-membered heterocycloalkyl, 5-10-membered heteroaryl and C₆₋₁₀aryl, the latter 4 groups and each C₁₋₁₀alkylene, C₂₋₁₀alkenylene and C₂₋₁₀alkynylene being optionally substituted with 1-4 substituents independently selected from halo, nitro, C₁₋₆alkyleneR^{c}, C₁₋₆alkyleneOR^{c}, OC₁₋₆alkyleneOR^{c}, C₁₋₆alkyleneNR^{c}R^{d}, OC₁₋₆alkyleneNR^{c}R^{d}, OR^{c}, NR^{c}R^{d}, CO₂R^{c}, CONR^{c}R^{d}, SO₂NR^{c}R^{d}, OCONR^{c}R^{d}, COR^{e}, C₃₋₁₀cycloalkyl, 3-10-membered heterocycloalkyl, 5-10-membered heteroaryl and C₆₋₁₀aryl;
R^{c} is selected from H, C₁₋₆alkyl, COC₁₋₆alkyl, C₃₋₁₀cycloalkyl, 3-10-membered heterocycloalkyl, 5-10-membered heteroaryl and C₆₋₁₀aryl;
R^{d} is selected from H and C₁₋₆alkyl;
R^{e} is selected from H, C₁₋₆alkyl, C₃₋₁₀cycloalkyl, 3-10-membered heterocycloalkyl, 5-10-membered heteroaryl and C₆₋₁₀aryl;
R² is selected from H, C₁₋₆alkyl, C₂₋₆alkenyl and C₂₋₆alkynyl;
Ring B is a 5 or 6-membered heteroaromatic ring comprising 1 to 3 heteroatoms selected from O, N and S;
R³ is selected from H, C₁₋₆alkyl, C₃₋₁₀cycloalkyl and 3-10-membered heterocycloalkyl, the latter three groups being optionally substituted with halo, OR^{f}, NR^{f}R^{g}, COR^{f}, CO₂R^{f}, CONR^{f}R^{g}, SO₂NR^{f}R^{g}, NHSO₂R^{f} and OCONR^{f}R^{g};
R^{f} and R^{g} are independently selected from H and C₁₋₆alkyl;
R⁴ is selected from H, C₁₋₆alkyl, C₂₋₆alkenyl, C₂₋₆alkynyl and C₃₋₇cycloalkyl;
R⁵ is selected from H, halo and C₁₋₆alkyl;
R⁶ is selected from COR^{h}, CO₂R^{h}, CONR^{h}Rⁱ, OCONR^{h}Rⁱ, NHSO₂R^{h}, SO₂NR^{h}Rⁱ, NHCOR^{h} and NHCO₂R^{h},
R^{h} and Rⁱ are independently selected from H and C₁₋₆alkyl;
R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₁₀cycloalkyl, 4-10-membered heterocycloalkyl, 5-10-membered heteroaryl and C₆₋₁₀aryl, the latter 4 groups being optionally substituted with 1-4 substituents independently selected from halo, nitro, C₁₋₆alkyleneR^{m}, C₁₋₆alkyleneOR^{m}, OC₁₋₆alkyleneOR^{m}, C₁₋₆alkyleneNR^{m}Rⁿ, OC₁₋₆alkyleneNR^{m}Rⁿ, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl, or R⁷ is 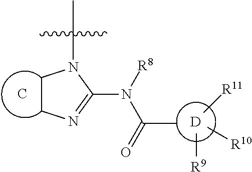
R^{j} is selected from H, C₁₋₆alkyl, COC₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, C₁₋₆alkyleneNR^{m}Rⁿ, OC₁₋₆alkyleneNR^{m}Rⁿ, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{k} is selected from H and C₁₋₆alkyl;
R^{l} is selected from H, C₁₋₆alkyl, C₃₋₁₀cycloalkyl, 4-10-membered heterocycloalkyl, 5-10-membered heteroaryl and C₆₋₁₀aryl, the latter 7 groups being optionally substituted with 1-4 substituents independently selected from halo, nitro, C₁₋₆alkyleneR^{m}, C₁₋₆alkyleneOR^{m}, OC₁₋₆alkyleneOR^{m}, C₁₋₆alkyleneNR^{m}Rⁿ, OC₁₋₆alkyleneNR^{m}Rⁿ, OR^{m}, NR^{m}Rⁿ, CO₂R^{m}, CONR^{m}Rⁿ, SO₂NR^{m}Rⁿ, OCONR^{m}Rⁿ, COR^{m}, C₃₋₁₀cycloalkyl, 4-10-membered heterocycloalkyl, 5-10-membered heteroaryl and C₆₋₁₀aryl;
R^{m} is selected from H and C₁₋₆alkyl;
Rⁿ is selected from H, C₁₋₆alkyl, C₃₋₁₀cycloalkyl, 4-10-membered heterocycloalkyl, 5-10-membered heteroaryl and C₆₋₁₀aryl;
R⁸, R⁹, R¹⁰ and R¹¹ are, independently, as defined for R², R³, R⁴ and R⁵, respectively;
Ring C is selected from phenyl and a 5 or 6-membered heteroaromatic ring comprising 1 to 3 heteroatoms selected from O, N and S, and Ring C is substituted with at least one R¹² and optionally substituted with one or two additional substituents independently selected from halo, C₁₋₆alkyl, C₂₋₆alkenyl, C₂₋₆alkynyl, OR^{o}, NR^{o}R^{p}, C₁₋₆alkyleneOR^{o}, C₁₋₆alkyleneNR^{o}R^{p}, OC₁₋₆alkyleneOR^{o}, OC₁₋₆alkyleneNR^{o}R^{p}, C₂₋₆alkenyleneOR^{o}, C₂₋₆alkenyleneNR^{o}R^{p}, OC₂₋₆alkenyleneOR^{o}, OC₂₋₆alkenyleneNR^{o}R^{p}, C₂₋₆alkynyleneOR^{o}, C₂₋₆alkynyleneNR^{o}R^{p}, OC₂₋₆alkynyleneOR^{o} and OC₂₋₆alkynyleneNR^{o}R^{p};
R^{o} and R^{p} are independently selected from H, C₁₋₆alkyl, C₁₋₆alkyleneOC₁₋₆alkyl, C₂₋₆alkenyleneOC₁₋₆alkyl, and C₂₋₆alkynyleneOC₁₋₆alkyl, or
R^{o} and R^{p} are joined to form, together with the atoms therebetween, a 3-10 membered saturated or unsaturated ring, optionally containing one additional heteroatom selected from NR^{t}, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R^{t} is selected from H, D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
Ring D is a 5 or 6-membered heteroaromatic ring comprising 1 to 3 heteroatoms selected from O, N and S;
R¹² is selected from COR^{q}, CO₂R^{q}, CONR^{q}R^{r}, OCONR^{q}R^{r}, NHSO₂R^{q}, SO₂NR^{q}R^{r}, NHCOR^{q} and NHCO₂R^{q};
R^{q} and R^{r} are independently selected from H and C₁₋₆alkyl;
and
each alkyl, alkenyl, alkynyl, alkylene, alkenylene and alkynylene group is optionally fluoro-substituted.

2. The compound of claim 1, wherein Ring A is pyridinyl or thienyl and is substituted with one R⁶ and R⁶ is CONR²⁵R²⁶, and/or Ring C is phenyl, pyridinyl or thienyl and is substituted with one R¹² and R¹² is CONR^{q}R^{r}.

3. The compound of claim 1 or claim 2, wherein Ring A and/or Ring C are independently substituted with one additional substituent selected from Cl, F, CH₃, CH₂CH₃, CF₂H, OCH₃, OCH₂CH₃, OH, CF₃, OCF₃, CF₂H, OCF₂H, NH₂, NHCH₃, CH₂OCH₃, CH₂NHCH₃, CH₂NH₂, CH₂OCF₃, CH₂OCF₂H, CH₂CH₂OCH₃, CH₂CH₂NHCH₃, CH₂CH₂NH₂, CH₂CH₂OCF₂H, CH₂CH₂OCF₃, OCH₂OCH₃, OCH₂NHCH₃, OCH₂NH₂, OCH₂OCF₃, OCH₂OCF₂H, OCH₂CH₂OCH₃, OCH₂CH₂NHCH₃, OCH₂CH₂NH₂, OCH₂CH₂CH₂NHCH₃, OCH₂CH₂OCF₂H and OCH₂CH₂OCF₃.

4. The compound of claim 1 or claim 2, wherein Ring A is substituted with one additional substituent selected from C₁₋₄alkyleneNR^{a}R^{b} and OC₁₋₄alkyleneR^{a}R^{b} and/or Ring C is substituted with one additional substituent selected from C₁₋₄alkyleneNR^{o}R^{p} and OC₁₋₄alkyleneR^{o}R^{p}, and each alkylene, is optionally fluoro-substituted

5. The compound of claim 4, wherein R^{a} is selected from H and C₁₋₆alkyl and R^{b} is selected from C₁₋₆alkyleneOC₁₋₆alkyl, C₂₋₆alkenyleneOC₁₋₆alkyl, and C₂₋₆alkynyleneOC₁₋₆alkyl, and/or R^{o} is selected from H and C₁₋₆alkyl and RP is selected from C₁₋₆alkyleneOC₁₋₆alkyl, C₂₋₆alkenyleneOC₁₋₆alkyl, and C₂₋₆alkynyleneOC₁₋₆alkyl, and each alkyl, alkylene, alkenylene and alkynylene group is optionally fluoro-substituted.

6. The compound of claim 4, wherein R^{a} and R^{b} are joined to form, together with the atoms therebetween, a 3-6 membered saturated or unsaturated ring or a 7-10 membered saturated or unsaturated ring, optionally containing one additional heteroatom selected from NR^{s}, O S, SO and SO₂, and/or
R^{o} and R^{p} are joined to form, together with the atoms therebetween, a 3-6 membered saturated or unsaturated ring or a 7-10 membered saturated or unsaturated ring optionally containing one additional heteroatom selected from NR^{t}, O, S, SO and SO₂.

7. The compound of claim 6, wherein R^{s} and R^{t} are independently selected from H, D, CHO, CO₂H, CONH₂, C₁₋₄alkyl, C₁₋₄alkenyl, C₁₋₄alkynyl, C(O)C₁₋₆alkyl, CO₂C₁₋₄alkyl, OC₁₋₄alkyl, CONHC₁₋₄alkyl, CON(C₁₋₄alkyl)(C₁₋₄alkyl), C₁₋₄alkyleneOC₁₋₄alkyl, C₂₋₄alkenyleneOC₁₋₄alkyl, and C₂₋₄alkynyleneOC₁₋₄alkyl, and each alkyl, alkenyl, alkynyl, alkylene, alkenylene and alkynylene group is optionally fluoro-substituted.

8. The compound of claim 6 or claim 7, wherein the 3-6 membered saturated or unsaturated ring or the 7-10 membered saturated or unsaturated ring is optionally substituted with D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl, and each alkyl, alkenyl, alkynyl, alkylene, alkenylene and alkynylene group is optionally fluoro-substituted.

9. The compound of any one of claims 1 to 8, wherein R¹ is selected from C₁₋₆alkyleneR⁷, C₂₋₆alkenyleneR⁷ and C₂₋₆alkynyleneR⁷; and each C₁₋₆alkylene, C₂₋₆alkenylene and C₂₋₆alkynylene is optionally substituted with 1-2 substituents independently selected from F, C₁₋₄alkyleneR^{c}, C₁₋₄alkyleneOR^{c}, OC₁₋₄alkyleneOR^{c}, OR^{c}, NR^{c}R^{d}, CO₂R^{c}, CONR^{c}R^{d}, SO₂NR^{c}R^{d}, OCONR^{c}R^{d} and COR^{e}; and R^{c}, R^{d} and R^{e} are independently selected from H and C₁₋₄alkyl, and each alkyl, alkylene, alkenylene and alkynylene group is optionally fluoro-substituted.

10. The compound of any one of claims 1 to 9 wherein R⁷ is selected from OR^{j}, NR^{j}R^{k}, NHCO₂R^{j}, NHCOR^{l}, CO₂R^{j}, COR^{l}, CONR^{j}R^{k}, OCONR^{j}R^{k}, NHSO₂R^{j}, SO₂NR^{j}R^{k}, C₃₋₆cycloalkyl, 4-6-membered heterocycloalkyl, 5-6-membered heteroaryl and phenyl, the latter 4 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m} and phenyl;
R^{j} is selected from H, C₁₋₄alkyl, COC₁₋₄alkyl, C₃₋₆cycloalkyl, 4-6-membered heterocycloalkyl, 5-6-membered heteroaryl and phenyl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m} and phenyl;
R^{k} is selected from H and C₁₋₄alkyl;
R^{l} is selected from H, C₁₋₄alkyl, C₃₋₆cycloalkyl, 4-6-membered heterocycloalkyl, 5-6-membered heteroaryl and phenyl, the latter 7 groups being optionally substituted with 1-2 substituents independently selected from Cl, F, C₁₋₄alkyleneR^{m}, C₁₋₄alkyleneOR^{m}, OC₁₋₄alkyleneOR^{m}, OR^{m}, NR^{m}Rⁿ, CO₂R^{m}, CONR^{m}Rⁿ, SO₂NR^{m}Rⁿ, OCONR^{m}Rⁿ, COR^{m} and phenyl;
R^{m} and Rⁿ are independently selected from H and C₁₋₄alkyl,
and each alkyl, alkenyl, alkynyl, alkylene, alkenylene and alkynylene group is optionally fluoro-substituted, or
R⁷ is 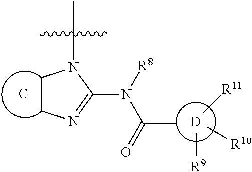
wherein R⁸, R⁹, R¹⁰ and R¹¹ are as defined in claim 1.

11. A compound of Formula II or a pharmaceutically acceptable salt, solvate and/or prodrug thereof: 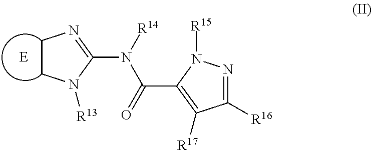
wherein:
Ring E is a 5 or 6-membered heteroaromatic ring comprising 1 to 3 heteroatoms selected from O, N and S that is substituted with at least one R¹⁸ and optionally substituted with one or two additional substituents independently selected from OC₁₋₆alkyleneNR¹⁹R²⁰, OC₂₋₆alkenyleneNR¹⁹R²⁰, OC₂₋₆alkynyleneNR¹⁹R²⁰, OC₁₋₆alkyleneNR²¹C₁₋₆alkyleneOC₁₋₆alkyl, OC₂₋₆alkenyleneNR²¹C₁₋₆alkyleneOC₁₋₆alkyl, OC₂₋₆alkynyleneNR²¹C₁₋₆alkyleneOC₁₋₆alkyl, OC₁₋₆alkyleneNR²¹C₂₋₆alkenyleneOC₁₋₆alkyl, OC₂₋₆alkenyleneNR²¹C₂₋₆alkenyleneOC₁₋₆alkyl, OC₂₋₆alkynyleneNR²¹C₂₋₆alkenyleneOC₁₋₆alkyl, OC₁₋₆alkyleneNR²¹C₂₋₆alkynyleneOC₁₋₆alkyl, OC₂₋₆alkenyleneNR²¹C₂₋₆alkynyleneOC₁₋₆alkyl, and OC₂₋₆alkynyleneNR²¹C₂₋₆alkynyleneOC₁₋₆alkyl;
R¹³ is selected from C₁₋₁₀alkyleneR²², C₂₋₁₀alkenyleneR²² and C₂₋₁₀alkynyleneR²²;
R¹⁴ is selected from H, C₁₋₆alkyl, C₂₋₆alkenyl and C₂₋₆alkynyl;
R¹⁵ is selected from H, C₁₋₆alkyl, C₃₋₁₀cycloalkyl and 3-10-membered heterocycloalkyl, the latter three groups being optionally substituted with halo, OR²³, NR²³R²⁴, COR²³, CO₂R²³, CONR²³R²⁴, SO₂NR²³R²⁴, NHSO₂R²³ and OCONR²³R²⁴;
R¹⁶ is selected from H, C₁₋₆alkyl, C₂₋₆alkenyl, C₂₋₆alkynyl and C₃₋₇cycloalkyl;
R¹⁷ is selected from H, halo and C₁₋₆alkyl;
R¹⁸ is selected from COR²⁵, CO₂R²⁵, CONR²⁵R²⁶, OCONR²⁵R²⁶, NHSO₂R²⁵, SO₂NR²⁵R²⁶, NHCOR²⁵ and NHCO₂R²⁵,
R¹⁹ and R²⁰ are joined to form, together with the nitrogen therebetween, a 3-6 membered saturated or unsaturated ring, optionally containing one additional heteroatom selected from NR²⁷, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl) or C₁₋₆alkyleneOC₁₋₆alkyl; or
R¹⁹ and R²⁰ are joined to form, together with the nitrogen therebetween, a 7-10 membered saturated or unsaturated ring, optionally containing one additional heteroatom selected from NR²⁷, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R²³, R²⁴, R²⁵ and R²⁶ are independently selected from H and C₁₋₆alkyl;
R²⁷ is selected from D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R²² is 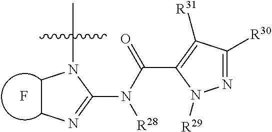
R²⁸, R²⁹, R³⁰ and R³¹ are, independently, as defined for R¹⁴, R¹⁵, R¹⁶ and R¹⁷, respectively;
Ring F is selected from phenyl and a 5 or 6-membered heteroaromatic ring comprising 1 to 3 heteroatoms selected from O, N and S, and Ring F is substituted with at least one R³² and substituted with one or two additional substituents independently selected from OC₁₋₆alkyleneNR³³R³⁴, OC₂₋₆alkenyleneNR³³R³⁴, OC₂₋₆alkynyleneNR³³R³⁴, OC₁₋₆alkyleneNR³⁵C₂₋₆alkenyleneOC₁₋₆alkyl, OC₁₋₆alkyleneNR³⁵C₁₋₆alkyleneOC₁₋₆alkyl, OC₂₋₆alkenyleneNR³⁵C₁₋₆alkyleneOC₁₋₆alkyl, OC₂₋₆alkynyleneNR³⁵C₁₋₆alkyleneOC₁₋₆alkyl, OC₁₋₆alkyleneNR³⁵C₂₋₆alkenyleneOC₁₋₆alkyl, OC₂₋₆alkenyleneNR³⁵C₂₋₆alkenyleneOC₁₋₆alkyl, OC₂₋₆alkynyleneNR³⁵C₂₋₆alkenyleneOC₁₋₆alkyl, OC₁₋₆alkyleneNR³⁵C₂₋₆alkynyleneOC₁₋₆alkyl, OC₂₋₆alkenyleneNR³⁵C₂₋₆alkynyleneOC₁₋₆alkyl, and OC₂₋₆alkynyleneNR³⁵C₂₋₆alkynyleneOC₁₋₆alkyl;
R³² is selected from COR³⁶, CO₂R³⁶, CONR³⁶R³⁷, OCONR³⁶R³⁷, NHSO₂R³⁶, SO₂NR³⁶R³⁷, NHCOR³⁶ and NHCO₂R³⁶;
R³³ and R³⁴ are joined to form, together with the nitrogen therebetween, a 3-6 membered saturated or unsaturated ring, optionally containing one additional heteroatom selected from NR³⁸, O, S, SO and SO₂, and substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl) or C₁₋₆alkyleneOC₁₋₆alkyl; or
R³³ and R³⁴ are joined to form, together with the nitrogen therebetween, a 7-10 membered saturated or unsaturated ring, optionally containing one additional heteroatom selected from NR³⁸, O, S, SO and SO₂, and optionally substituted with one or more of D, OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
R³⁵, R³⁶, and R³⁷ are independently selected from H and C₁₋₆alkyl;
R³⁸ is selected from D, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl;
and
each alkyl, alkenyl, alkynyl, alkylene, alkenylene and alkynylene group is optionally fluoro-substituted.

12. The compound of claim 11, wherein Ring E is pyridinyl or thienyl and is substituted with one R¹⁸ and R¹⁸ is CONR²⁵R²⁶, and/or Ring F is phenyl, pyridinyl or thienyl and is substituted with one R³² and R³² is CON³⁶R³⁷.

13. The compound of claim 11 or claim 12 wherein Ring E is optionally substituted with one additional substituent selected from OC₁₋₄alkyleneNR¹⁹R²⁰, OC₂₋₄alkenyleneNR¹⁹R²⁰, OC₂₋₄alkynyleneNR¹⁹R²⁰, OC₁₋₄alkyleneNR²¹C₁₋₄alkyleneOC₁₋₄alkyl, OC₂₋₄alkenyleneNR²¹C₁₋₄alkyleneOC₁₋₄alkyl, OC₂₋₄alkynyleneNR²¹C₁₋₄alkyleneOC₁₋₄alkyl, OC₁₋₄alkyleneNR²¹C₂₋₄alkenyleneOC₁₋₄alkyl, OC₂₋₄alkenyleneNR²¹C₂₋₄alkenyleneOC₁₋₆alkyl, OC₂₋₄alkynyleneNR²¹C₂₋₄alkenyleneOC₁₋₄alkyl, OC₁₋₄alkyleneNR²¹C₂₋₄alkynyleneOC₁₋₄alkyl, OC₂₋₄alkenyleneNR²¹C₂₋₄alkynyleneOC₁₋₄alkyl, and OC₂₋₄alkynyleneNR²¹C₂₋₄alkynyleneOC₁₋₄alkyl, and/or
Ring F is substituted with one additional substituent selected from OC₁₋₄alkyleneNR³³R³⁴, OC₂₋₄alkenyleneNR³³R³⁴, OC₂₋₄alkynyleneNR³³R³⁴, OC₁₋₄alkyleneNR³⁵C₁₋₄alkyleneOC₁₋₄alkyl, OC₂₋₄alkenyleneNR³⁵C₁₋₄alkyleneOC₁₋₄alkyl, OC₂₋₄alkynyleneNR³⁵C₁₋₄alkyleneOC₁₋₄alkyl, OC₁₋₄alkyleneNR³⁵C₂₋₄alkenyleneOC₁₋₄alkyl, OC₂₋₄alkenyleneNR³⁵C₂₋₄alkenyleneOC₁₋₆alkyl, OC₂₋₄alkynyleneNR²¹C₂₋₄alkenyleneOC₁₋₄alkyl, OC₁₋₄alkyleneNR³⁵C₂₋₄alkynyleneOC₁₋₄alkyl, OC₂₋₄alkenyleneNR³⁵C₂₋₄alkynyleneOC₁₋₄alkyl, and OC₂₋₄alkynyleneNR²¹C₂₋₄alkynyleneOC₁₋₄alkyl,
and each alkyl, alkenyl, alkynyl, alkylene, alkenylene and alkynylene group is optionally fluoro-substituted.

14. The compound of claim 13, wherein R¹⁹ and R²⁰ are joined to form, together with the atoms therebetween, a 3-6 membered saturated or unsaturated ring or a 7-10 membered saturated or unsaturated ring, optionally containing one additional heteroatom selected from NR²⁷, O S, SO and SO₂, and/or
R³⁴ and R³⁵ are joined to form, together with the atoms therebetween, a 3-6 membered saturated or unsaturated ring or a 7-10 membered saturated or unsaturated ring optionally containing one additional heteroatom selected from NR³⁸, O, S, SO and SO₂.

15. The compound of claim 14, wherein the 3-6 membered saturated or unsaturated ring or the 7-10 membered saturated or unsaturated ring is substituted with one or more of OH, NH₂, CHO, CO₂H, CONH₂, C₁₋₆alkyl, C₁₋₆alkenyl, C₁₋₆alkynyl, C(O)C₁₋₆alkyl, CO₂C₁₋₆alkyl, OC₁₋₆alkyl, CONHC₁₋₆alkyl, CON(C₁₋₆alkyl)(C₁₋₆alkyl), C₁₋₆alkyleneOC₁₋₆alkyl, C₂₋₆alkenyleneOC₁₋₆alkyl, and C₂₋₆alkynyleneOC₁₋₆alkyl, suitably one or more of D, CHO, CO₂H, CONH₂, C₁₋₆alkyl, C(O)C₁₋₆alkyl, CO₂C₁₋₆alkyl, OC₁₋₆alkyl, C₁₋₆alkyleneOC₁₋₆alkyl, C₂₋₆alkenyleneOC₁₋₆alkyl, and C₂₋₆alkynyleneOC₁₋₆alkyl, more suitably one or more of D, CHO, C₁₋₆alkyl, C(O)C₁₋₆alkyl, OC₁₋₆alkyl, or C₁₋₆alkyleneOC₁₋₆alkyl, and each alkyl, alkenyl, alkynyl, alkylene, alkenylene and alkynylene group is optionally fluoro-substituted.

16. A compound of claim 1, selected from:

| Compound ID | Structure |
|---|---|
| A | 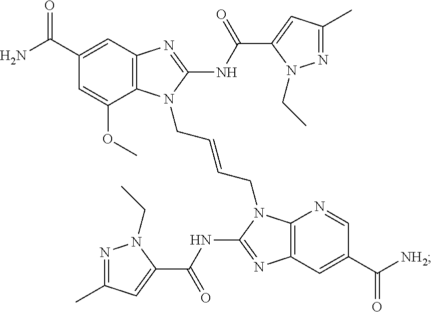 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OC)c21] |
| B | 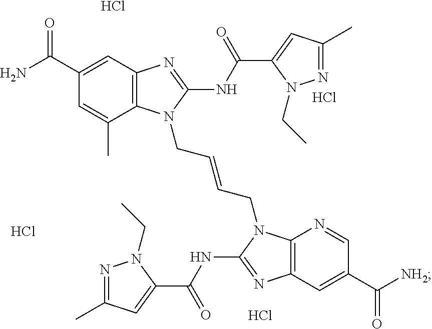 [SMILES: Cl ; Cl ; CCn1nc(C)cc1C(=O)Nc1nc2cc(C(N)=O)cnc2n1C/C=C/Cn1c(NC(=O)c2cc(C)nn2CC)nc2cc(C(N)=O)cc(C)c21 ; Cl ; Cl] |
| C | 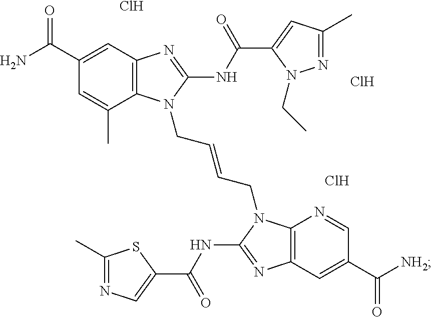 [SMILES: Cl ; CCn1nc(C)cc1C(=O)Nc1nc2cc(C(N)=O)cc(C)c2n1C/C=C/Cn1c(NC(=O)c2cnc(C)s2)nc2cc(C(N)=O)cnc21 ; Cl ; Cl] |
| D | 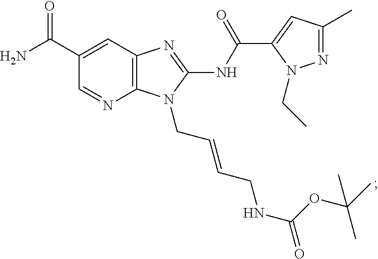 [SMILES: CCn1nc(C)cc1C(=O)Nc1nc2cc(C(N)=O)cnc2n1C/C=C/CNC(=O)OC(C)(C)C] |
| E | 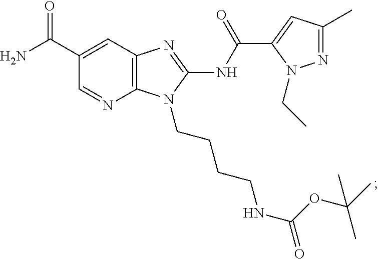 [SMILES: CCn1nc(C)cc1C(=O)Nc1nc2cc(C(N)=O)cnc2n1CCCCNC(=O)OC(C)(C)C] |
| F | 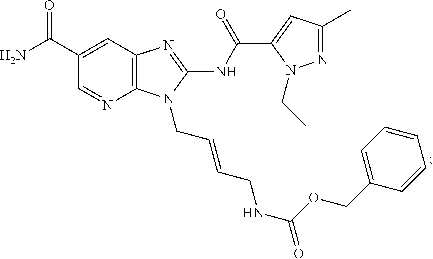 [SMILES: CCn1nc(C)cc1C(=O)Nc1nc2cc(C(N)=O)cnc2n1C/C=C/CNC(=O)OCc1ccccc1] |
| G | 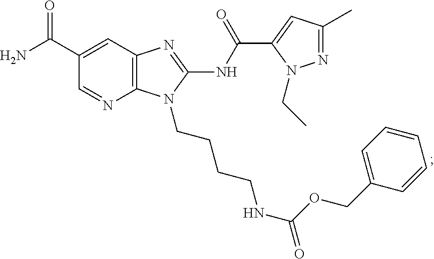 [SMILES: CCn1nc(C)cc1C(=O)Nc1nc2cc(C(N)=O)cnc2n1CCCCNC(=O)OCc1ccccc1] |
| H | 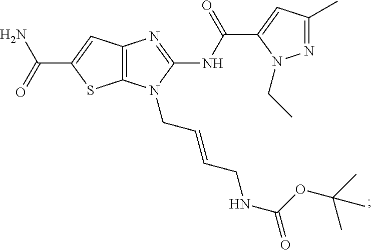 [SMILES: CCn1nc(C)cc1C(=O)Nc1nc2cc(C(N)=O)sc2n1C/C=C/CNC(=O)OC(C)(C)C] |
| J | 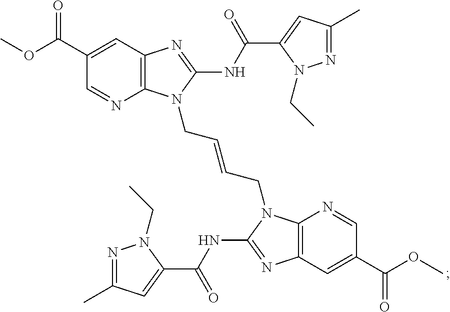 |
| K | 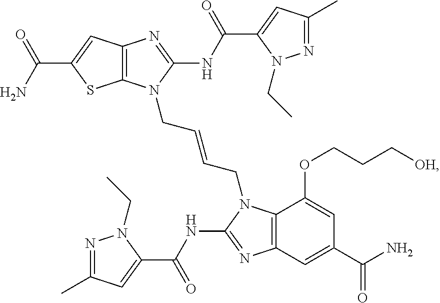 [SMILES: CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCO)c21] |
| L | 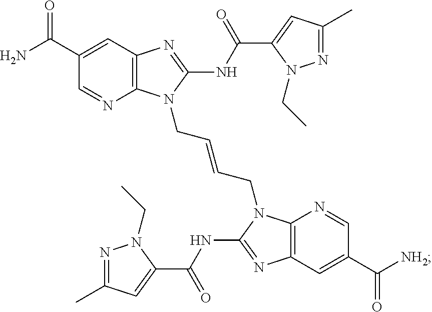 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cnc21] |
| M | 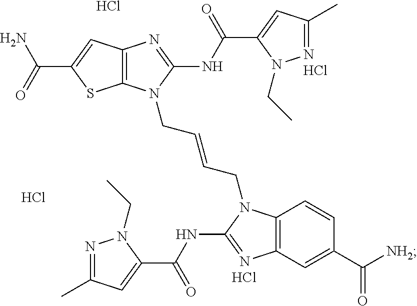 [SMILES: CCn1nc(C)cc1C(=O)Nc1nc2cc(C(N)=O)ccc2n1C/C=C/Cn1c(NC(=O)c2cc(C)nn2CC)nc2cc(C(N)=O)sc21 ; Cl ; Cl ; Cl ; Cl] |
| N | 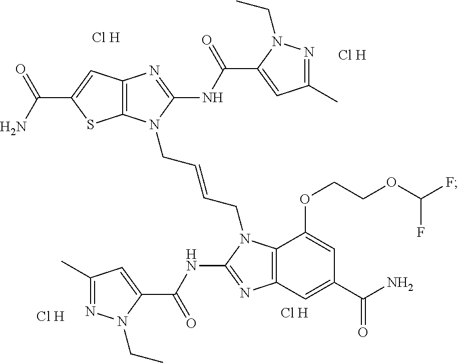 |
| O | 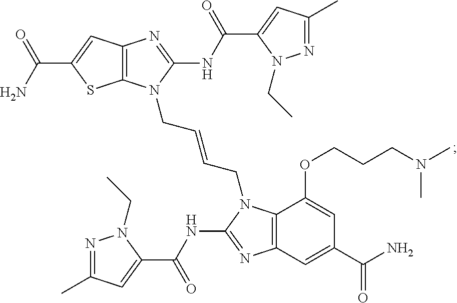 [SMILES: CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCN(C)C)c21] |
| P | 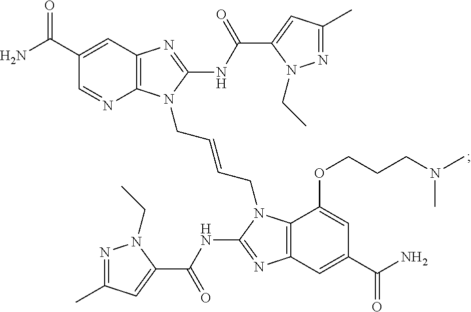 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN(C)C)c21] |
| Q | 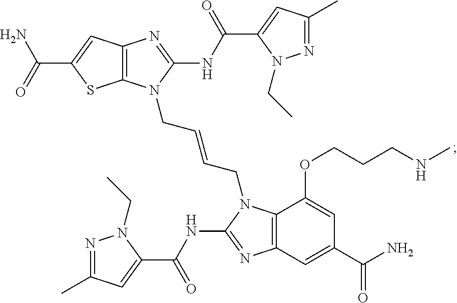 [SMILES: CCn1nc(C)cc1C(=O)Nc1nc2cc(C(N)=O)sc2n1C/C=C/Cn1c(NC(=O)c2cc(C)nn2CC)nc2cc(C(N)=O)cc(OCCCNC)c21] |
| R | 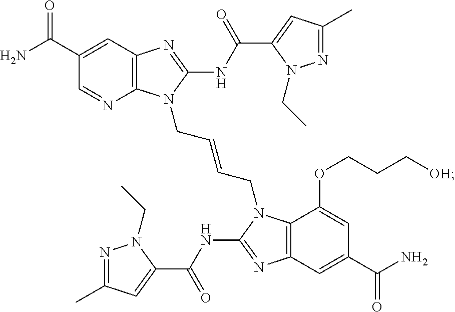 |
| S | 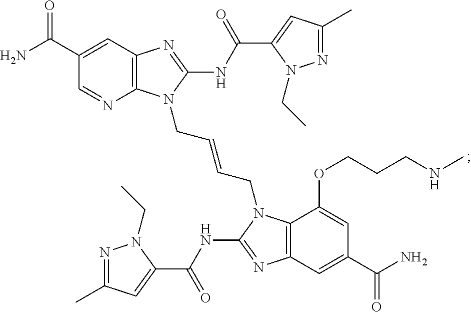 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNC)c21] |
| T | 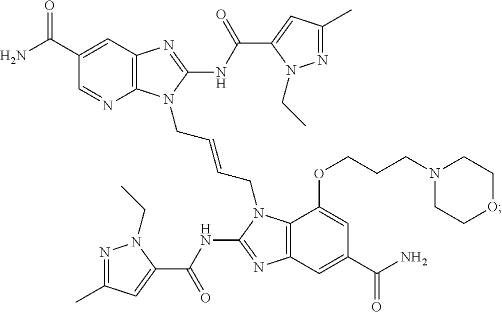 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCOCC3)c21] |
| U | 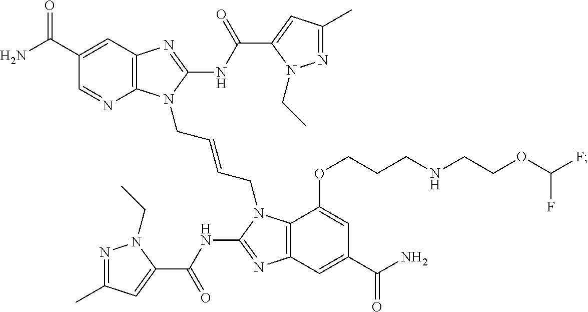 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCNCCOC(F)F)c21] |
| V | 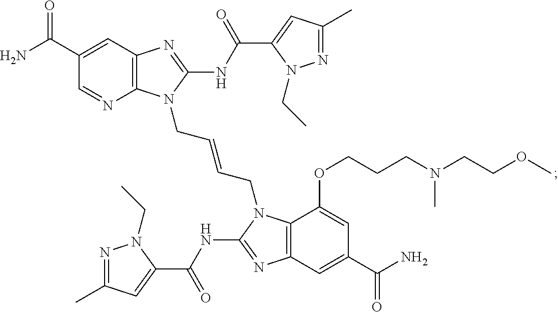 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN(C)CCOC)c21] |
| W | 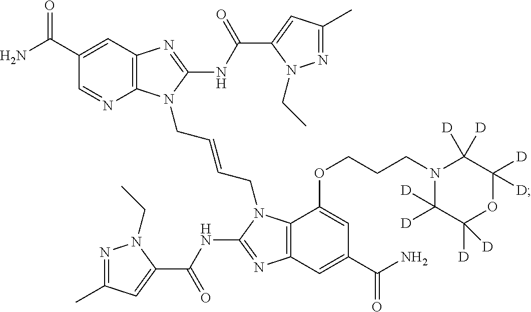 |
| X | 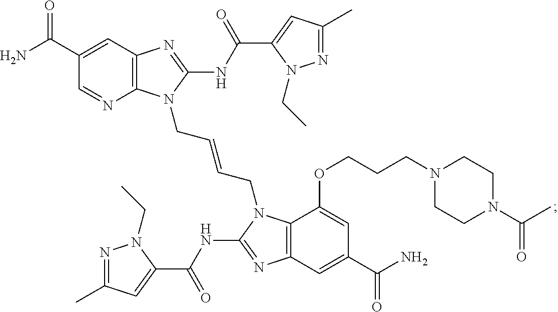 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCN(C(C)=O)CC3)c21] |
| Y | 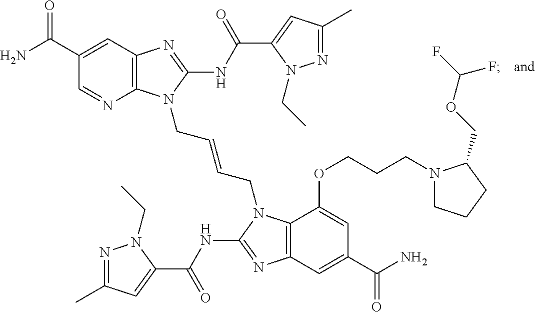 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCC[C@H]3COC(F)F)c21] |
| Z | 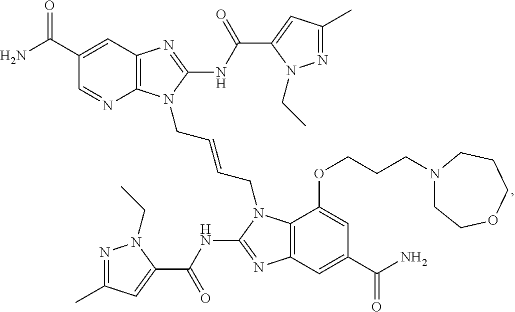 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCN3CCCOCC3)c21] |

or a pharmaceutically acceptable salt, solvate and/or prodrug thereof.

17. The compound of any one of claims 1 to 16 wherein one or more of the compounds are coupled to an antibody to form an antibody-drug conjugate.

18. A composition comprising one or more compounds of any one of claims 1 to 16 and a carrier.

19. A method of treating a disease, disorder or condition treatable by activation of STING, or mediated by STING protein activation comprising administering a therapeutically effective amount of one or more compounds of any one of claims 1 to 16 to a subject in need thereof.

20. The method of claim 19 wherein the disease, disorder or condition mediated by STING protein activation, or treatable by activation of STING, is cancer.
